SEQUENCE LISTING (14 sequences)

SEQ ID NO: 1 - protein, 8 aa, synthetic construct
GYTFTSYY

SEQ ID NO: 2 - protein, 8 aa, synthetic construct
INPSNAGT

SEQ ID NO: 3 - protein, 12 aa, synthetic construct
TREGNPYYTMNY

SEQ ID NO: 4 - protein, 11 aa, synthetic construct
QSLLESDGKTY

SEQ ID NO: 5 - skipped

SEQ ID NO: 6 - protein, 9 aa, synthetic construct
WQGTHFPWT

SEQ ID NO: 7 - protein, 119 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 8 - protein, 112 aa, synthetic construct
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIK

SEQ ID NO: 9 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 10 aa, synthetic construct
FLSRPTEKTI

(19) United States
(12) Patent Application Publication
(10) Pub. No.: US 2024/0391987 A1
(43) Pub. Date: Nov. 28, 2024

(54) METHOD OF TREATING BONE CANCERS

(71) Applicant: AlaMab Therapeutics, Inc., Princeton, NJ (US)

(72) Inventors: Yanfeng Zhang, Phoenixville, PA (US); Xiugao Yang, Shanghai (CN); Yongyong Wu, Beijing (CN)

(21) Appl. No.: 18/673,081

(22) Filed: May 23, 2024

Related U.S. Application Data
(60) Provisional application No. 63/504,162, filed on May 24, 2023.

(51) Int. Cl. C07K 16/18; A61K 39/00; A61P 35/00

(52) U.S. Cl. CPC C07K 16/18; A61P 35/00; A61K 2039/505; C07K 2317/24; C07K 2317/75

(57) ABSTRACT

The present disclosure provides compositions and methods of treating bone cancers, e.g., osteosarcoma in patients who previously failed at least one line of therapy for treatment of osteosarcoma. The method comprises promoting the opening of Cx43 hemichannel in osteocytes by, e.g., using a composition comprising an anti-connexin 43 antibody.

### CROSS-REFERENCE TO RELATED APPLICATION

This application claims priority to U.S. Provisional Application Ser. No. 63/504,162, filed on May 24, 2023, the disclosure of which is hereby incorporated by reference in its entirety.

### INCORPORATION OF SEQUENCE LISTING

The instant application contains a Sequence Listing, which has been submitted via Patent Center. The Sequence Listing titled 172628-202003_US_SL.xml, which was created on May 8, 2024, and is 14,454 bytes in size, is hereby incorporated by reference in its entirety.

### BACKGROUND

Osteosarcoma is the most common primary malignant bone tumor derived from primitive bone-forming mesenchymal stem cells. Osteosarcoma has a bimodal age distribution, with the first peak in adolescents aged 10-14 years and the second peak in adults aged >65 years. It generally occurred more in males than in females, 5.4/million/year in men vs. 4.0/million/year in women, and more in black people (6.8/million) and Hispanics (6.5/million) than in white people (4.6/million).

Osteosarcoma usually occurs in the long bones of the extremities close to the bony end of the metaphyseal growth plate. Of all children and adolescent cancer death, 8.9% died from bone and joint malignancies. The 5-year survival rate for osteosarcoma was 68%, without significant gender differences. The expected 5-year survival rate for metastatic patients is 15%-30% (Lin et al., Trends in molecular medicine, 23(8): 737-755 (2017)), and the survival rate of patients is age-related, with older patients having a lower survival rate. The prognosis of osteosarcoma is very poor. According to statistics, the 5-year and 10-year overall survival is 0.23 and 0.18, respectively (Kempf-Bielack et al., J Clin Oncol., 23: 559-568 (2005)). Tumor staging, metastasis, local recurrence, chemotherapy regimen, anatomical location, tumor size, and tumor cell destruction rate after neoadjuvant chemotherapy are all associated with the results of treatment (Ottaviani et al., Cancer Treat Res., 152: 3-13 (2009)).

For high-grade osteosarcoma (including intramedullary and surface forms), preoperative chemotherapy is recommended (Grade 1A), followed by reassessment and re-staging by chest radiography, local X-ray, PET, or bone scan. For resectable tumors, wide resection should be performed. When the resection margin is negative and the response to chemotherapy is good, chemotherapy should be continued; when the response to chemotherapy is poor, the chemotherapy regimen can be modified. When the resection margin is positive and the response to chemotherapy is good, chemotherapy should be continued, and other local treatments (surgery, radiotherapy, etc.) may be performed concomitantly; when the response to chemotherapy is poor, chemotherapy regimen can be modified, and other local treatments (surgery, radiotherapy, etc.) should be performed concomitantly. For unresectable tumors, only radiotherapy and chemotherapy are considered. Patients should be continuously monitored after treatment.

Osteocytes have high expression of connexin Cx43 in interstitial junction channels and hemichannels. The gap junction channels mediate cellular communication between two adjacent cells to maintain the normal metabolic function of the cells, while the hemichannels allow molecular exchange between the cells and the extracellular environment. Hemichannels are normally closed, but under certain conditions, such as mechanical stress or ischemia, cytokine or ion stimulation, they will be activated and opened. Opened hemichannels allow small molecules (<1.2 kDa) such as ATP, NAD+, glutamic acid, and inositol triphosphate (IP3) to pass (Goodenough et al., Nature reviews Molecular cell biology, 4: 285-294 (2003)). It has been confirmed that the opening of the Cx43 hemichannels of osteocytes has a strong inhibitory effect on the growth, migration and bone metastasis of breast cancer tumors (Zhou et al., Oncogene, 35: 5597-5607 (2016)).

Current first-line chemotherapeutics for osteosarcoma are methotrexate, doxorubicin, cisplatin, and ifosfamide. As recommended by CSCO in 2020, since there is no second-line treatment regimen showing overall survival benefit, participation in clinical studies is an opportunity for osteosarcoma patients who fail first-line treatments to achieve better efficacy or to renew treatment. It has been reported in the literature that a drug is considered effective in clinical studies if it provides a response rate of >5% or a 4-month PFS rate of >40%. Systemic chemotherapy is the primary treatment for advanced osteosarcoma, and no major breakthrough has been made in the treatment of advanced osteosarcoma in the past decades. As of now, there remains a large and unmet need for medication in advanced osteosarcoma.

### SUMMARY

One aspect of the present disclosure provides a method of treating a bone cancer in a subject in need thereof, comprising administering to the subject at least one dose of an anti-connexin 43 (Cx43) antibody, or antigen binding fragment thereof, the anti-Cx43 antibody comprises a heavy chain variable region comprising: HCDR1 amino acid sequence comprising SEQ ID NO: 1, HCDR2 amino acid sequence comprising SEQ ID NO: 2, and HCDR3 amino acid sequence comprising SEQ ID NO: 3; and a light chain variable region comprising: LCDR1 amino acid sequence comprising SEQ ID NO: 4, LCDR2 amino acid sequence comprising SEQ ID NO: 5, and LCDR3 amino acid sequence comprising SEQ ID NO: 6.

In some embodiments, the at least one dose of the anti-Cx43 antibody, or antigen binding fragment thereof, is about 0.001 mg/kg to about 300 mg/kg. In some specific embodiments, the at least one dose of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

In some embodiments, the anti-Cx43 antibody, or antigen binding fragment thereof, is administered about once every day to about once every 12 months. In some specific embodiments, the anti-Cx43 antibody, or antigen binding fragment thereof, is administered about once every 2 weeks, about once every 3 weeks, or about once every 4 weeks. In one embodiment, the anti-Cx43 antibody, or antigen binding fragment thereof, is administered about once every 3 weeks.

In some embodiments, the anti-Cx43 antibody, or antigen binding fragment thereof, is administered intravenously, intradermally, intratumorally, intramuscularly, intraperitoneally, or subcutaneously. In a specific embodiment, the anti-Cx43 antibody, or antigen binding fragment thereof, is administered intravenously.

In some embodiments, the anti-Cx43 antibody, or antigen binding fragment thereof, is administered within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

In some embodiments, the anti-Cx43 antibody, or antigen binding fragment thereof, is administered in a pharmaceutically acceptable composition. In some embodiments, the composition comprises a pharmaceutically acceptable carrier.

In some embodiments, the anti-Cx43 antibody, or antigen binding fragment thereof, comprises a heavy chain variable region comprising amino acid sequence according to SEQ ID NO: 7, and/or a light chain variable region comprising amino acid sequence according to SEQ ID NO: 8.

In some embodiments, the anti-Cx43 antibody comprises a heavy chain comprising amino acid sequence according to any one of SEQ ID NOs: 9, and 11-13, and/or a light chain comprising amino acid sequence according to SEQ ID NO: 10.

In some embodiments, the anti-Cx43 antibody is a humanized antibody.

In some embodiments, the anti-Cx43 antibody, or antigen binding fragment thereof, enhances Cx43 hemichannel opening in the subject.

In some embodiments, the bone cancer is osteosarcoma.

In some embodiments, the osteosarcoma is resectable, while in other embodiments, the osteosarcoma is unresectable.

In some embodiments, the osteosarcoma is stage I-A, stage I-B, stage II-A, stage II-B, stage III, stage IV-A, or stage IV-B.

In some embodiments, the osteosarcoma is low grade, whereas in other embodiments, the osteosarcoma is high grade.

In some embodiments, the osteosarcoma is primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

In some embodiments, the subject had failed treatment with at least one standard care of osteosarcoma comprising chemotherapy, surgery, immune therapy, or radiotherapy, or a combination thereof.

In some embodiments, the subject had failed treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

In some embodiments, after treatment with the anti-Cx43 antibody, or antigen binding fragment thereof, the subject achieves: (i) progression-free survival for at least 1 months, 2 months, 3 months, at least 4 months, at least 5 months, or at least 6 months; (ii) improved overall survival by at least 1 month, at least 2 months, at least 3 months, at least 4 months, at least 5 months, at least 6 months; (iii) improved EQ-5D score; (iv) improved disease progression as assessed by PERCIST criteria, RECIST criteria, and/or ICDS criteria; (v) improved pain score as assessed by NRS; and/or (vi) reduced tumor size by at least 1%, at least 2%, at least 3%, at least 5%, at least 8%, at least 10%, at least 15%, at least 20%, or at least 30%.

In some embodiments, the subject achieves complete or partial response to the treatment 15 with the anti-Cx43 antibody.

A further aspect of the present disclosure provides a method of treating a bone cancer in a subject in need thereof, comprising administering to the subject at least one dose of an anti-connexin 43 (Cx43) antibody, or antigen binding fragment thereof, wherein the at least one dose of the anti-Cx43 antibody is about 0.01 mg/kg to about 300 mg/kg.

In some embodiments, the at least one dose of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

In some embodiments, the anti-Cx43 antibody, or antigen binding fragment thereof, is administered about once every day to about once every 12 months. In some specific embodiments, the anti-Cx43 antibody, or antigen binding fragment thereof, is administered about once every 2 weeks, about once every 3 weeks, or about once every 4 weeks. In one embodiment, the anti-Cx43 antibody, or antigen binding fragment thereof, is administered about once every 3 weeks.

In some embodiments, the anti-Cx43 antibody, or antigen binding fragment thereof, is administered intravenously, intradermally, intratumorally, intramuscularly, intraperitoneally, subcutaneously, or locally. In one embodiment, the anti-Cx43 antibody, or antigen binding fragment thereof, is administered intravenously.

In some embodiments, the anti-Cx43 antibody, or antigen binding fragment thereof, is administered within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

In some embodiments, the anti-Cx43 antibody, or antigen binding fragment thereof, is administered in a pharmaceutically acceptable composition. In some embodiments, the pharmaceutically acceptable composition comprises a pharmaceutically acceptable carrier.

In some embodiments, the anti-Cx43 antibody, or antigen binding fragment thereof, comprises a heavy chain variable region comprising: HCDR1 amino acid sequence comprising SEQ ID NO: 1, HCDR2 amino acid sequence comprising SEQ ID NO: 2, and HCDR3 amino acid sequence comprising SEQ ID NO: 3; and a light chain variable region comprising: LCDR1 amino acid sequence comprising SEQ ID NO: 4, LCDR2 amino acid sequence comprising SEQ ID NO: 5, and LCDR3 amino acid sequence comprising SEQ ID NO: 6.

In some embodiments, the anti-Cx43 antibody, or antigen binding fragment thereof, comprises a heavy chain variable region comprising amino acid sequence according to SEQ ID NO: 7, and/or a light chain variable region comprising amino acid sequence according to SEQ ID NO: 8.

In some embodiments, the anti-Cx43 antibody comprises a heavy chain comprising amino acid sequence according to any one of SEQ ID NOs: 9, and 11-13, and/or a light chain comprising amino acid sequence according to SEQ ID NO: 10.

In some embodiments, the anti-Cx43 antibody is a humanized antibody.

In some embodiments, the anti-Cx43 antibody, or antigen binding fragment thereof, enhances Cx43 hemichannel opening in the subject.

In some embodiments, the bone cancer is osteosarcoma.

In some embodiments, the osteosarcoma is resectable, while in some other embodiments, the osteosarcoma is unresectable.

In some embodiments, the osteosarcoma is stage I-A, stage I-B, stage II-A, stage II-B, stage III, stage IV-A, or stage IV-B.

In some embodiments, the osteosarcoma is low grade; while in some other embodiments, the osteosarcoma is high grade.

In some embodiments, the osteosarcoma is primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

In some embodiments, the subject had failed treatment with at least one standard of care of osteosarcoma comprising chemotherapy, surgery, radiotherapy, or immune therapy, or the combination thereof.

In some embodiments, the subject had failed treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

In some embodiments, after treatment with the anti-Cx43 antibody, or antigen-binding fragment thereof, the subject achieves: (i) progression-free survival for at least 1 months, 2 months, 3 months, at least 4 months, at least 5 months, or at least 6 months; (ii) improved overall survival by at least 1 month, at least 2 months, at least 3 months, at least 4 months, at least 5 months, at least 6 months; (iii) improved EQ-5D score; (iv) improved disease progression as assessed by PERCIST criteria, RECIST criteria, and/or ICDS criteria; (v) improved pain score as assessed by NRS; and/or (vi) reduced tumor size by at least 1%, at least 2%, at least 3%, at least 5%, at least 8%, at least 10%, at least 15%, at least 20%, or at least 30%.

In some embodiments, the subject achieves complete or partial response to the treatment with the anti-Cx43 antibody, or antigen-binding fragment thereof.

### BRIEF DESCRIPTION OF DRAWINGS

FIG. 1 shows the pain grades of Numeric Rating Scale.

FIG. 2 shows the VAS scale of the EQ-5D questionnaire.

FIG. 3 is the swim lane plot showing the investigator-determined overall efficacy of the 24 enrolled participants. Participant 502001 received 1 mg/kg ALMB-0168 per dose; participant 502002 received 3 mg/kg ALMB-0168 per dose; participants SO1001, SO1002 and 502003 received 6 mg/kg ALMB-0168 per dose; participants 502006 and 506001 received 12 mg/kg ALMB-0168 per dose; participants S02007, 506002 and 506008 received 18 mg/kg ALMB-0168 per dose; participants S02010, 509001 and S11003 received 24 mg/kg ALMB-0168 per dose; participants S02012, S06004, S01003 and S03001 received 30 mg/kg ALMB-0168 per dose; participants S01004 and S06005 received 36 mg/kg ALMB-0168 per dose; upright triangle indicates partial response (PR), circle indicates stable disease (SD); diamond indicates progressive disease (PD); right-pointing triangle indicates trial ongoing; cross indicates end of study (EOS); pentagon indicates starting new anti-cancer therapy; plus indicates death.

FIG. 4 is the waterfall plot showing the investigator-determined best change of target lesion vs baseline (Efficacy Evaluable Analysis Set).

FIG. 5 is the left knee prosthesis visible at baseline in participant SO1001; recurrent metastases was observed in right upper femur.

FIGS. 6A-6F are the Participant cross-section image along the long diameter of participant SO1001: baseline image (FIG. 6A) collected on Oct. 28, 2021, and the first 5 tumor evaluation images collected on Dec. 20, 2021 (FIG. 6B), Feb. 23, 2022 (FIG. 6C), Apr. 19, 2022 (FIG. 6D), Jun. 27, 2022 (FIG. 6E) and Aug. 29, 2022 (FIG. 6F), respectively. The participant achieved partial response per RECIST 1.1 and bone density increase.

FIG. 7 shows images of participant SO1001: baseline image (first panel) collected on Oct. 28, 2021, and 4 tumor evaluation images collected on Dec. 20, 2021 (second panel), Feb. 23, 2022 (third panel), Apr. 19, 2022 (fourth), and Aug. 29, 2022 (fifth panel), respectively. The first panel: the mean legion size is about 187.45, standard deviation is about 128.3, the area is 11.02 cm², the maximum length ≥706 unit, the minimum length ≤−252; the second panel: the mean legion size is about 476.07 unit, standard deviation is about 292.39 unit, the area is 6.9 cm², the maximum length ≥1391 unit, the minimum length ≤−252 unit; the third panel: the mean legion size is about 796.28 unit, standard deviation is about 457 unit, the area is 5.621 cm², the maximum length ≥1954 unit, the minimum length ≤−344 unit; the fourth panel: the mean legion size is about 1009.3 unit, standard deviation is about 398.84 unit, the area is 5.533 cm², the maximum length ≥1697 unit, the minimum length ≤−115 unit; the fifth panel: the mean legion size is about 970.93 unit, standard deviation is about 23 unit, the area is 4.594 cm², the maximum length ≥1320 unit, the minimum length ≤49 unit.

FIGS. 8A-8B are images showing legion 1 (FIG. 8A) and legion 2 (FIG. 8B) of participant S02007 collected at baseline (left panel) and post-treatment (right panel). The length of legion 1 at baseline as shown in FIG. 8A, left panel is 3.19 cm; the length of legion 2 at baseline as shown in FIG. 8A, right panel is 2.09 cm; the length of legion 1 post-treatment as shown in FIG. 8B, left panel is 1.94 cm; the length of legion 2 post-treatment as shown in FIG. 8B, right panel is 1.41 cm.

### DETAILED DESCRIPTION

The present disclosure provides methods and compositions for treating a cancer, e.g., bone cancer, in a subject or patent in need thereof, comprising administering at least one dose of an anti-Cx43 antibody to the subject or patent. In some embodiments, the anti-Cx43 antibody promotes the opening of Cx43 hemichannel. In some embodiments, the anti-Cx43 antibody comprises specific CDR amino acid sequences. In some embodiments, the anti-Cx43 antibody is administered according to a dosing regimen.

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “providing” is used according to its ordinary meaning to supply or furnish for use. In some embodiments, the protein (e.g., an antibody) is provided directly by administering the protein, while in other embodiments, the protein (e.g., an antibody) is effectively provided by administering a nucleic acid that encodes the protein. In certain aspects the invention contemplates compositions comprising various combinations of nucleic acid, antigens, peptides, and/or epitopes.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST.

The term “pharmaceutical formulation” or “pharmaceutical composition” refers to a preparation that contains a therapeutic agent (e.g., an anti-Cx43 antibody). In such form as to permit the biological activity of the antibody to be effective, and which contains no additional components which are unacceptably toxic to a subject to which the formulation would be administered.

As used herein, the term “antigen” is a molecule capable of being bound by an antibody or T-cell receptor. In certain embodiments, binding moieties other than antibodies are engineered to specifically bind to an antigen, e.g., aptamers, avimers, and the like.

As used herein, the term “specifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹. Antibodies may, for example, bind with an EC so of 50 nM or less, 10 nM or less, 1 nM or less, 100 pM or less, or more preferably 10 pM or less. As kwon in the art, a variety of immunoassay formats may be used to select antibodies specifically immunoreactive with an antigen. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

An “affinity matured” antibody has one or more alterations in one or more hypervariable regions thereof which result an improvement in the affinity of the antibody for antigen, compared to a parent antibody which does not possess those alteration(s). In one aspect, affinity matured antibodies will have nanomolar or even picomolar affinities for the target antigen. Affinity matured antibodies are produced by procedures known in the art. Marks et al., Bio Technology, 10:779-783 (1992) describes affinity maturation by VH and VL domain shuffling. Random mutagenesis of CDR and/or framework residues is described by: Barbas et al., Proc Nat. Acad. Sci. USA, 91:3809-3813 (1994); Schier et al., Gene, 169: 147-155 (1995); Yelton et al., J. Immunol., 155:1994-2004 (1995); Jackson et al., J. Immunol., 154(7):3310-9 (1995); and Hawkins et al., J. Mo. Biol., 226:889-896 (1992).

“Cancer” or “tumor” is intended to include any malignant or neoplastic growth in a patient, including an initial tumor and any metastases. The cancer can be of the hematological or solid tumor type. Hematological tumors include tumors of hematological origin, including, e.g., myelomas (e.g., multiple myeloma), leukemias (e.g., Waldenstrom's syndrome, chronic lymphocytic leukemia, acute myelogenous leukemia, chronic myelogenous leukemia, granulocytic leukemia, monocytic leukemia, acute lymphocytic leukemia, other leukemias), lymphomas (e.g., B-cell lymphomas, such as diffuse large B-cell lymphoma, follicular lymphoma, mantle cell lymphoma, Hodgkin's lymphoma, non-Hodgkin's lymphoma, plasmocytoma, or reticulum cell sarcoma), and myeloproliferative neoplasms, such as myelodysplastic syndrome, thrombocythemia, polycythemia vera, or myelofibrosis. Solid tumors can originate in organs, and include cancers such as in bone, skin, lung, brain, breast, prostate, ovary, colon, kidney, pancreas, liver, esophagus, stomach, intestine, bladder, uterus, cervix, testis, adrenal gland, etc. As used herein, cancer cells, including tumor cells, refer to cells that divide at an abnormal (increased) rate or whose control of growth or survival is different than for cells in the same tissue where the cancer cell arises or lives. Cancer cells include, but are not limited to, cells in carcinomas, sarcomas, myelomas, leukemias, lymphomas, and tumors of the nervous system including glioma, meningoma, medulloblastoma, schwannoma or epidymoma. In a preferred embodiment, the cancer is bone cancer. In a more preferred embodiment, the bone cancer is osteosarcoma.

The term “affinity” refers to the strength of a binding reaction between a binding domain of an antibody and an epitope. It is the sum of the attractive and repulsive forces operating between the binding domain and the epitope. The term affinity, as used herein, refers to the dissociation constant KD.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In one embodiment, an epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping and hydrogen-deuterium exchange. Epitopes can be formed both from contiguous amino acids or noncontiguous amino acids juxtaposed by tertiary folding of a protein. Epitopes formed from contiguous ammo acid are typically retained on exposure to denaturing solvents, whereas epitopes formed by tertiary folding are typically lost on treatment with denaturing solvents. An epitope typically includes at least 3, and more usually, at least 5 or 8-10 amino acid in a unique spatial conformation.

The terms “promote,” “enhance” and “induce” as used interchangeably herein, refer to any statistically significant increase in biological activity (e.g., hemichannel opening). For example, “promotion” can refer to an increase of about 10%, 20%, 30%, 40%, 50%, 60%, 70%, 80%, 90%, or 100% in biological activity.

The term “bone cancer” or “bone tumor” as used herein, refers to any cancer related to bone, including a cancer originated from a bone, or a cancer originated from er organs or tissues, e.g., (breast, prostate, lung, kidney, and thyroid etc.) and metastasizes to a bone. Accordingly, non-limiting examples of a bone cancer include osteosarcoma, multiple myeloma, Ewing's sarcoma, osteoporosis, osteopenia, osteoblastoma, chondroma, chondromyxoid fibroma, mesenchymal chondrosarcoma, chordoma, adamantinoma, or bone cancer metastasis.

Osteosarcoma is the most common primary malignant bone tumor. By definition, osteosarcoma is an osteoid-forming spindle cell neoplasm with several histologic subtypes demonstrating variable radiographic appearances, intrinsic cellular heterogeneity, and overall prognosis. Subtypes of osteosarcoma include but are not limited to: osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, a chondromyxoid fibroma-like osteosarcoma, clear-cell osteosarcoma, malignant fibrous histiocytoma-like osteosarcoma, giant cell rich osteosarcoma, or epithelioid osteosarcoma. he World Health Organizations considers osteoblastic, chondroblastic and fibroblastic subtypes of conventional intramedullary osteosarcoma, the most common type of osteosarcoma.

Osteoblastic osteosarcoma arises from abnormal proliferation of osteoblasts. Osteoblastic osteosarcoma typically develops in the metaphyseal region of long bones, such as the distal femur, proximal tibia, proximal humerus etc, but it can also occur in other bones. Osteoblastic osteosarcoma is characterized in the setting of abundant extracellular osteoid production. The matrix comprised of immature bone is deposited in a lace-like pattern with malignant tumor cells incorporated throughout the extracellular matrix.

Chondroblastic osteosarcoma as defined by WHO is a histological entity characterized by predominant presence of chondroid matrix, which tends to exhibit a high degree of hyaline cartilage and is intimately associated with the nonchondroid element (osteoid or bone matrix). Chondroblastic osteosarcoma typically occurs in the long bones, such as the femur and tibia. It can also affect other bones, including the humerus and pelvis. Chondroblastic osteosarcoma is more common in adolescents and young adults, but it can occur at any age.

Fibroblastic osteosarcomas are characterized by the presence of spindle-shaped cells resembling fibroblasts. Fibroblastic osteosarcomas are highly vascular and may resemble hemangiopericytoma. In histopathology, osteoid production is minimal and seen focally.

Small cell osteosarcoma, characterized by the presence of small, round, and uniform cells within the tumor, is a rare subtype of osteosarcoma. In histopathology, small cell osteosarcoma cells have scant cytoplasm and large, hyperchromatic nuclei. small cell osteosarcoma mainly occurs in the metaphyseal region of long bones.

Benign giant cells resembling osteoclasts are seen in about 25% of osteosarcomas. In rare osteosarcomas, benign giant cells are so numerous that they may obscure the malignant cells in the background and lead to the mistaken diagnosis of a giant cell tumor. This is more likely to occur in sacrum—a frequent location for giant cell tumor. The radiographic differences between the giant cell tumor and osteosarcoma are less apparent in the sacrum than in the long bones. It is worth remembering that usual giant cell tumor occurs in skeletally mature patients. A tumor that appears to be a giant cell tumor but arising in a skeletally immature person should be sampled thoroughly and carefully to exclude osteosarcoma rich in giant cells.

There are many classification systems for grading osteosarcoma. One of the widely used system is the 4-tiered (Broder's) system based on the percentage of anaplasia in the tumor. Majority of the conventional central osteosarcomas fall in grade 3 or grade 4 category. The tumors assigned grade 1 are low-grade central osteosarcomas and the usual parosteal osteosarcomas. Osteosarcomas of the jaw often tend to show grade 2 histology.

The common staging systems for malignant bone tumors are: Enneking system for staging malignant musculoskeletal tumors and the American Joint Committee on Cancer (AJCC) System for staging osteosarcomas. Enneking system is based on histological grade of the tumor, its local extent and the presence or absence of metastasis. The AJCC system for bone sarcomas is based on tumor grade, size, presence, and location of metastases. Different stages of the AJCC system are shown below.

| Stage | Grade | Size | Metastasis |
|---|---|---|---|
| I-A | Low | <8 cm | None |
| I-B | Low | >8 cm | None |
| II-A | High | <8 cm | None |
| II-B | High | >8 cm | None |
| III | Any | Any | Skip metastasis |
| IV-A | Any | Any | Pulmonary metastasis |
| IV-B | Any | Any | Non-Pulmonary metastasis |

### II. Antibodies

The term “antibody” or “antibodies” herein is used in the broadest sense and specifically covers full length antibody, antibody peptide(s) or immunoglobulin(s), monoclonal antibodies, chimeric antibodies, polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multispecific antibodies (e.g., bispecific antibodies) formed from at least two full length antibodies (e.g., each portion comprising the antigen binding region of an antibody to a different antigen or epitope), and individual antigen binding fragments of any of the foregoing, e.g., of an antibody or the antibody from which it is derived, including dAbs, Fv, scFv, Fab, F(ab)′2, Fab′.

Antibody-like binding peptidomimetics are also contemplated in the methods described herein. Liu et al. (2003) describe “antibody-like binding peptidomimetics” (ABiPs), which are peptides that act as pared-down antibodies and have certain advantages of longer serum half-life as well as less cumbersome synthesis methods.

The term “monoclonal antibody” as used herein refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

“Antigen binding fragments” of an antibody preferably comprise at least the variable regions of the heavy and/or light chains of an anti-Cx43 antibody. For example, an antigen binding fragment of anti-Cx43 antibodies can comprise amino acid sequences of SEQ ID NOs: 7 and 8. Examples of such antigen binding fragments include Fab fragments, Fab′ fragments, Fv fragments, scFv and F(ab′)2 fragments. Antigen binding fragments of an antibody can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a recombinant construct encoding the heavy chain of an F(ab′)2 fragment can be designed to include DNA sequences encoding the CH1 domain and hinge region of the heavy chain. In one aspect, antigen binding fragments promote opening of Cx43 hemi-channel in a subject and the effects associated with opening of Cx43 hemi-channel.

A “therapeutic monoclonal antibody” is an antibody used for therapy of a human subject. Therapeutic monoclonal antibodies disclosed herein include anti-Cx43 antibodies. Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), and the like. To assess ADCC activity of a molecule of interest, an in vitro ADCC assay, such as those described in U.S. Pat. Nos. 5,500,362 or 5,821,337 may be performed.

Depending on the amino acid sequence of the constant domain of their heavy chains, full length antibodies can be assigned to different “classes.” There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of antibodies are well known. The “light chains” of antibodies from any vertebrate species can be assigned to one of two clearly distinct types, called kappa (κ) and lambda (λ), based on the amino acid sequences of their constant domains.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “hypervariable region” when used herein refers to the amino acid residues of an antibody which are responsible for antigen binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mal. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

As will be appreciated by those in the art, the CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

Amino acid substitutions are typically of single residues; insertions usually will be on the order of from about one to about twenty amino acid residues, although considerably larger insertions may be tolerated. Deletions range from about one to about twenty amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

The term “Fab” or “Fab region,” as used herein, is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

The term “Fv,” “Fv fragment” or “Fv region,” as used herein, refers to a polypeptide that comprises the VL and VH domains of a single antibody.

The term “framework,” as used herein, is refers to the region of an antibody variable domain exclusive of those regions defined as CDRs. Each antibody variable domain framework can be further subdivided into the contiguous regions separated by the CD Rs (FR1, FR2, FR3 and FR4).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

### Anti-Cx43 Antibodies

Certain aspects of the present disclosure provide compositions and methods of treating a bone cancer, e.g., osteosarcoma. The methods and compositions comprise an anti-Cx43 antibody which specifically binds Cx43 hemichannel and promotes the opening of it in osteocytes.

The anti-Cx43 antibody can be any antibody specifically binds Cx43 known in the art. In various embodiments, the anti-Cx43 antibody used in the methods comprises an HCDR1 amino acid sequence having at least 90%, at least 95%, or at least 99% sequence identity to SEQ ID NO: 1, an HCDR2 amino acid sequence having at least 90%, at least 95%, or at least 99% sequence identity to SEQ ID NO: 2, and an HCDR3 amino acid sequence having at least 90%, at least 95%, or at least 99% sequence identity to SEQ ID NO: 3; and/or a LCDR1 amino acid sequence having at least 90%, at least 95%, or at least 99% sequence identity to SEQ ID NO: 4, a LCDR2 amino acid sequence having at least 90%, at least 95%, or at least 99% sequence identity to SEQ ID NO: 5, a LCDR3 amino acid sequence having at least 90%, at least 95%, or at least 99% sequence identity to SEQ ID NO: 6. In some specific embodiments, the anti-Cx43 antibody comprises an HCDR1 amino acid sequence identical to SEQ ID NO: 1, an HCDR2 amino acid sequence identical to SEQ ID NO: 2, and an HCDR3 amino acid sequence identical to SEQ ID NO: 3; and/or a LCDR1 amino acid sequence identical to SEQ ID NO: 4, a LCDR1 amino acid sequence identical to SEQ ID NO: 5, and a LCDR3 amino acid sequence identical to SEQ ID NO: 6.

In various embodiments, the anti-Cx43 antibody used in the method comprises a heavy chain variable region having at least 80%, at least 85%, at least 90%, at least 95%, or at least 99% sequence identity to SEQ ID NO: 7; and/or a light chain variable region having at least 80%, at least 85%, at least 90%, at least 95%, or at least 99% sequence identity to SEQ ID NO: 8. In some specific embodiments, the anti-Cx43 antibody comprises a heavy chain variable region identical to SEQ ID NO: 7; and/or a light chain variable region identical to SEQ ID NO: 8.

In various embodiments, the anti-Cx43 antibody used in the method comprise a heavy chain having at least 80%, at least 85%, at least 90%, at least 95%, or at least 99% sequence identity to any one of SEQ ID NOs: 9 and 11-13; and/or a light chain having at least 80%, at least 85%, at least 90%, at least 95%, or at least 99% sequence identity to any one of SEQ ID NO: 10. In some specific embodiments, the anti-Cx43 antibody comprise a heavy chain having at least 80%, at least 85%, at least 90%, at least 95%, or at least 99% sequence identity to SEQ ID NO: 9; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 11; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 12; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 13; and a light chain having at least 80%, at least 85%, at least 90%, at least 95%, or at least 99% sequence identity to any one of SEQ ID NO: 10.

In some embodiments, the anti-Cx43 antibody comprises a heavy chain identical to any one of SEQ ID NOs: 9 and 11-13, and/or a light chain identical to any one of SEQ ID NO: 10. In a preferred embodiment, the anti-Cx43 antibody comprises a heavy chain identical to SEQ ID NO: 9, and a light chain identical to any one of SEQ ID NO: 10. In one embodiment, the anti-Cx43 antibody comprises a heavy chain identical to SEQ ID NO: 11, and a light chain identical to any one of SEQ ID NO: 10. In another embodiment, the anti-Cx43 antibody comprises a heavy chain identical to SEQ ID NO: 12, and a light chain identical to any one of SEQ ID NO: 10. In yet another embodiment, the anti-Cx43 antibody comprises a heavy chain identical to SEQ ID NO: 13, and a light chain identical to any one of SEQ ID NO: 10.

In various embodiments, the anti-Cx43 antibody can comprise a mixture, or cocktail, of two or more anti-Cx43 antibodies, each of which binds to the same or different epitope on Cx43.

In various embodiments, provided herein is an antibody that binds an epitope located within, partially or entirely, the amino acid sequence of FLSRPTEKTI (SEQ ID NO: 14). in some embodiments, the epitope can comprise one or more amino acids selected from the group consisting of F1, S3, R4, P5, T6, E7, K8, T9, and I10 of SEQ ID NO: 14. In one embodiment the epitope consists of F1, S3, R4, P5, T6, E7, K8, T9 and 110 of SEQ ID NO: 14. In some embodiments, the epitope can include all ten amino acids of SEQ ID NO: 14. In certain embodiments, the epitope consists of all ten amino acids of SEQ ID NO: 14.

The anti-Cx43 antibody is substantially pure and desirably substantially homogeneous (i.e. free from contaminating proteins etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-Cx43 antibody, based on total weight of the protein.

In some embodiments, the anti-Cx43 antibody is a humanized antibody. In some embodiments, the anti-Cx43 antibody is a monoclonal antibody. In some a humanized monoclonal antibody.

### Pharmaceutical Formulation Comprising Anti-Cx43 Antibody

One aspect of the present disclosure provides a pharmaceutical formulation for treating cancer, e.g., a bone cancer including osteosarcoma comprising an anti-Cx43 antibody. In some 30 embodiments, the anti-Cx43 antibody promoting opening of Cx43 hemichannels in osteocytes. The anti-Cx43 antibody can be a full antibody or an antigen-binding fragment thereof.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent may be coated m a material to protect the compm.md from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can he brought about by including in the composition, m agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol phenol sorbic acid, and the like. it may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

It may be advantageous to formulate parenteral compositions of unit dosage form for ease of administration and uniformity of dosage. Unit dosage form as used herein refers to physically discrete units suited as unitary dosages for the patients to be treated: each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitations, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

The formulations described herein are administered to a mammal in need of treatment with the anti-Cx43 antibodies, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

In some embodiments, the formulations are administered to the mammal by intravenous or subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

### III. Method of Treating Bone Cancer

One aspect of the present disclosure provides methods of treating a cancer, e.g., bone cancer including osteosarcoma. The methods can comprise promoting/enhancing opening of Cx43 hemichannels in osteocytes in a subject in need thereof by using, e.g., an anti-Cx43 antibody. Currently there is a lack of second-line therapy for osteosarcoma, therefore, Cx43 modulation (e.g., promoting the opening of Cx43 hemichannel using an anti-Cx43 antibody) can be used as a second line therapy for those who failed standard care of osteosarcoma, or at least one line of therapy for treating osteosarcoma. The Cx43 modulation can be a stand-alone therapy for bone cancer including osteosarcoma, or in combination with other cancer therapies described herein or known in the art.

In some embodiments, the anti-Cx43 antibody is used as a second line therapy in a subject who failed at least one line of therapy (standard care) of a bone cancer (e.g., osteosarcoma). In some other embodiments, the anti-Cx43 antibody is used as a first line therapy in a subject for treatment of bone cancer (e.g., osteosarcoma). In some embodiments, the anti-Cx43 antibody is used as a neoadjuvant treatment of bone cancer (e.g., osteosarcoma) in a subject.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Normal bone is made up of three major cell types: bone-forming osteoblasts, bone resorbing osteoclasts, and osteocytes. Osteocytes make up approximately 95% of bone cells and maintain the bone remodeling process by coordinating osteolytic and osteoblastic activities. When cancer cells invade the bone, many of the normal bone functions are affected. Cancer cells interact with the local microenvironment to promote cancer cell survival via bone destruction and vascularization.

Osteocytes express hemichannels known as connexin (Cx) 43 hemichannels. These osteocyte hemichannels are normally closed and can be opened when exposed to mechano-stimulation, which leads to the release of various factors into the bone microenvironment. The factors released by hemichannel opening can mediate other processes that can decrease tumor cell migration and bone metastasis. Alendronate (AD), an efficacious and commonly used bisphosphonate drug has been shown to be able to open Cx43 hemichannels in osteocytes. Bisphosphonates are a class of drugs known for treating many bone disorders including bone metastasis. It has been shown that administration of bisphosphonates to be associated with a decrease in the incidence of bone metastasis and a decrease in death rate in patients with breast cancer. AD has been associated with decreased tumor growth as well as reduced bone destruction and pain. AD inhibits osteoclast activity and induces the opening of Cx43 hemichannels in osteocytes. However, AD administration is accompanied by multiple, severe side-effects.

Cx43 is also known as gap junction alpha-1 protein (GJA1), which is a 43.0 kDa protein composed of 382 amino acids (NCBI Reference Sequence: NP 000156.1). GJA1 contains a long C-terminal tail, an N-terminal domain, and multiple transmembrane domains. The protein passes through the phospholipid bilayer four times, leaving its C- and N-terminals exposed to the cytoplasm. The C-terminal tail is composed of 50 amino acids and includes post-translational modification sites, as well as binding sites for transcription factors, cytoskeleton elements, and other proteins. As a result, the C-terminal tail is central to functions such as regulating pH gating and channel assembly. Notably, the DNA region of the GJA1 gene (NCBI Gene ID: 2697) encoding this tail is highly conserved, indicating that it is either resistant to mutations or becomes lethal when mutated. Meanwhile, the N terminal domain is involved in channel gating and oligomerization and, thus, may control the switch between the channel's open and closed states. The transmembrane domains form the gap junction channel while the extracellular loops facilitate proper channel docking. Moreover, two extracellular loops form disulfide bonds that interact with two hexamers to form a complete gap junction channel.

“Treatment” refers to therapeutic treatment. Those in need of treatment include those already with disease. Hence, the patient, e.g., human, to be treated herein may have been diagnosed as suffering from a disease, such as bone cancer. A disease, e.g., bone cancer, is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

The term “subject” or “patient” refers to either a human or non-human, such as primates, mammals, and vertebrates. In particular embodiments, the subject is a human.

Treatment can be suitably administered to subjects, particularly humans, suffering from, having, susceptible to, or at risk of developing such cancer, e.g., bone cancer including osteosarcoma. Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g., opinion) or objective (e.g., measurable by a test or diagnostic method).

The anti-Cx43 antibody can be stored as a lyophilized solid or an aqueous formulation, or any other forms known in the art. In the case of an anti-Cx43 antibody which is stored as a lyophilized solid, the antibody is reconstituted in a solution such as water for injection prior to administration. If prepared for infusion either from a lyophilized form or an aqueous formulation, the final concentration, e.g., after dilution of the reconstituted antibody (e.g., in a saline, Ringer's or 5% dextrose infusion system) of the anti-Cx43 antibody can be about 0.1 mg/ml to about 80 mg/ml for administration. The final concentration may be about 0.1 mg/ml to about 80 mg/ml, about 0.5 mg/ml to about 70 mg/ml, about 1 mg/ml to about 60 mg/ml, about 5 mg/ml to about 50 mg/ml, about 10 mg/ml to about 40 mg/ml, about 15 mg/ml to about 30 mg/ml, or about 20 mg/ml to about 25 mg/ml. In some embodiments, the final dosage form may be at a concentration of less than 0.1 mg/ml, about 0.1 mg/ml, about 0.5 mg/ml, about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml or higher than 80 mg/ml.

The term “effective amount,” as used herein, refers to that amount of an agent, such as an anti-Cx43 antibody, which is sufficient to effect treatment, prognosis or diagnosis of a cancer (e.g., bone cancer), when administered to a patient or a subject. Dosage regimens may be adjusted to provide the optimum therapeutic response. An effective amount is also one in which any toxic or detrimental effects (side effects) of the agent are minimized and/or outweighed by the beneficial effects. A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art. For example, an effective amount or a dose of the anti-Cx43 antibody ranges from about 0.001 mg/kg to about 300 mg/kg. In some embodiments, a dose of the anti-Cx43 antibody is about 0.01 mg/kg to about 200 mg/kg, about 0.1 mg/kg to about 100 mg/kg, about 0.2 mg/kg to about 90 mg/kg, about 0.5 mg/kg to about 80 mg/kg, about 0.8 mg/kg to about 70 mg/kg, about 1 mg/kg to about 60 mg/kg, about 3 mg/kg to about 50 mg/kg, about 6 mg/kg to about 40 mg/kg, about 9 mg/kg to about 36 mg/kg, about 12 mg/kg to about 33 mg/kg, about 15 mg/kg to about 30 mg/kg, about 18 mg/kg to about 27 mg/kg, or about 21 mg/kg to about 24 mg/kg. In some embodiments, a dose of the anti-Cx43 antibody is about 0.001 mg/kg, about 0.01 mg/kg, about 0.1 mg/kg, about 1 mg/kg, about 3 mg/kg, about 6 mg/kg, about 9 mg/kg, about 12 mg/kg, about 15 mg/kg, about 18 mg/kg, about 21 mg/kg, about 24 mg/kg, about 27 mg/kg, about 30 mg/kg, about 33 mg/kg, about 36 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, or more than 300 mg/kg.

In some specific embodiments, the effect amount or a dose of the anti-Cx43 antibody is 1 mg/kg, 3 mg/kg, 6 mg/kg, 9 mg/kg, 12 mg/kg, 15 mg/kg, 18 mg/kg, 21 mg/kg, 24 mg/kg, 27 mg/kg, 30 mg/kg, 33 mg/kg, or 36 mg/kg.

In some embodiments, an effective amount or a dose of the anti-Cx43 antibody can range from about 1 mg to about 25,000 mg. In some embodiments, a dose of the anti-Cx43 antibody is about 2 mg to 20,000 mg, about 5 mg to 15,000 mg, about 10 mg to 10,000 mg, about 15 mg to 10,000 mg, about 20 mg to 5000 mg, about 25 mg to 4000 mg, about 30 mg to 3000 mg, about 40 mg to 2000 mg, about 50 mg to 1000 mg, about 60 mg to 900 mg, about 70 mg to 800 mg, about 80 mg to 700 mg, about 90 mg to 600 mg, about 100 mg to 500 mg, about 150 mg to 450 mg, about 200 mg to 400 mg, about 250 mg to 350 mg, or about 275 mg to 325 mg. In some embodiments, a dose of the anti-Cx43 antibody is less than 1 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1250 mg, about 1500 mg, about 1750 mg, about 2000 mg, about 2250 mg, about 2500 mg, about 2750 mg, about 3000 mg, about 4000 mg, about 5000 mg, about 6000 mg, about 7000 mg, about 8000 mg, about 9000 mg, about 10,000 mg, about 12,500 mg, about 15,000 mg, about 17,500 mg, about 20,000 mg, about 22,500 mg, about 25,000 mg, or more than 25,000 mg.

In the methods as described herein, an effective amount or a dose of the anti-Cx43 antibody is administered about once every day, about once every 2 days, about once every 3 days, about once every 4 days, about once every 5 days, about once every 6 days, about once every week, about once every 8 days, about once every 9 days, about once every 10 days, about once every 11 days, about once every 12 days, about once every 13 days, about once every 2 weeks, about once every 15 days, about once every 16 days, about once every 17 days, about once every 18 days, about once every 19 days, about once every 20 days, about once every 3 weeks, about once every 22 days, about once every 23 days, about once every 24 days, about once every 25 days, about once every 26 days, about once every 27 days, about once every 4 weeks, about once every 29 days, about once every 30 days, about once every 31 days, about once every 32 days, about once every 33 days, about once every 34 days, about once every 5 weeks, about once every 36 days, about once every 37 days, about once every 38 days, about once every 39 days, about once every 40 days, or about once every 41 days, about once every 6 weeks, about once every 7 weeks, about once every 8 weeks, about once every 9 weeks, about once every 10 weeks, about once every 11 weeks, about once every 12 weeks, about once every 13 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks or 6 months, or about once every more than 24 weeks or 6 months.

In some specific embodiments, a dose of the anti-Cx43 antibody is administered about once every 3 weeks. In one particular embodiment, a dose of the anti-Cx43 antibody is administered once every 3 weeks.

According to certain embodiments of the present disclosure, multiple doses of an anti-Cx43 antibody (or a pharmaceutical composition comprising a combination of an anti-Cx43 antibody and any of the additional therapeutically active agents mentioned herein) may be administered to a subject over a defined time course. The methods according to this aspect of the disclosure comprises sequentially administering to a subject multiple doses of an anti-Cx43 antibody of the disclosure.

As used herein, “sequentially administering” means that each dose of anti-Cx43 antibody is administered to the subject at a different point in time, e.g., on different days separated by a predetermined interval (e.g., hours, days, weeks or months). The present disclosure includes methods which comprise sequentially administering to the patient a single initial dose of an anti-Cx43 antibody, followed by one or more secondary doses of the anti-Cx43 antibody, and optionally followed by one or more tertiary doses of the anti-Cx43 antibody. The anti-Cx43 antibody may be administered at a dose of between 0.001 mg/kg to about 300 mg/kg.

The terms “initial dose,” “secondary doses,” and “tertiary doses,” refer to the temporal sequence of administration of the anti-Cx43 antibody of the disclosure. Thus, the “initial dose” is the dose which is administered at the beginning of the treatment regimen (also referred to as the “baseline dose”); the “secondary dose” are the doses which are administered after the initial dose; and the “tertiary doses” are the doses which are administered after the secondary doses.

The initial, secondary, and tertiary doses may all contain the same amount of anti-Cx43 antibody, but generally may differ from one another in terms of frequency of administration. In certain embodiments, however, the amount of anti-Cx43 antibody contained in the initial, secondary, tertiary and/or doses following the tertiary dose varies from one another (e.g., adjusted up or down as appropriate) during the course of treatment.

The anti-Cx43 antibody is suitably administered to the patient at one time or over a series of treatments and may be administered to the patient at any time from diagnosis onwards. The anti-Cx43 antibody may be administered as the sole treatment or in conjunction with other drugs or therapies useful in treating the condition in question.

In the methods as described herein, a dose of the anti-Cx43 antibody can be administered to a patient over less than 5 minutes, about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more than 180 minutes. In some specific embodiments, a dose of the anti-Cx43 antibody can be administered to a patient over about 30 minutes. In one specific embodiment, a dose of the anti-Cx43 antibody can be intravenously administered to a patient over 30 minutes.

In the methods described herein, an anti-Cx43 antibody is administered to a patient. If the anti-Cx43 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g. intravenous, intradermal, intramuscular, intratumoral, intraperitoneal or subcutaneous injection. In one specific embodiment, the In another aspect, a solid or dry formulation can be administered topically, e.g., in a patch, cream, aerosol or suppository.

In the methods described herein, the anti-Cx43 antibody may be administered to a subject alone or in conjunction with another agent. The anti-Cx43 antibody can be administered before, along with or subsequent to administration of the additional agent. In one embodiment, the dose of the co-administered agent can be decreased over time or completely tapered during the period of treatment by the anti-Cx43 antibody. Non-limiting examples the co-administered agent include chemotherapeutic drugs, radiotherapy, immune therapy and surgery.

Non-limiting examples of such chemotherapeutic drugs include platinum-based chemotherapy drugs (e.g., cisplatin, carboplatin), taxanes (e.g., paclitaxel (Taxol®), docetaxel (Taxotere®), EndoTAG-PM (a formulation of paclitaxel encapsulated in positively charged lipid-based complexes; MediGene), Abraxane® (a formulation of paclitaxel bound to albumin)), tyrosine kinase inhibitors (e.g., imatinib/Gleevec®, sunitinib/Sutent®, dasatinib/Sprycel®), and combinations thereof.

In another embodiment, the at least one additional anti-cancer agent comprises an EGFR inhibitor, such as an anti-EGFR antibody or a small molecule inhibitor of EGFR signaling. An exemplary anti-EGFR antibody is cetuximab (Erbituxl®) Cetuximab is commercially available from ImClone Systems Incorporated. Other examples of anti-EGFR antibodies include matuzumab (EMD72000), panitumumab (Vectibix®; Amgen); nimotuzumab (TheraCIM™) and mAb 806. An exemplary small molecule inhibitor of the EGFR signaling pathway is gefitinib (Iressa@), which is commercially available from AstraZeneca and Teva. Other examples of small molecule inhibitors of the EGFR signaling pathway include erlotinib HCL (OSI-774: Tarceva®), OSI Pharma); lapatinib (Tykerb®, GlaxoSmithKline); canertinib (canertinib dihydrochloride, Pfizer): pelitinib (Pfizer): PKI-166 (Novartis); PD158780; and AG 1478 (4-(3-Chloroanillino)-6,7-dimethoxyquinazoline).

In yet another embodiment, the at least one additional anti-cancer agent comprises a VEGF inhibitor. An exemplary VEGF inhibitor comprises an anti-VEGF antibody, such as bevacizumab (Avastatin@: Genentech). In still another embodiment, the at least one additional anti-cancer agent comprises an anti-ErbB2 antibody. Suitable anti-ErbB2 antibodies include trastuzumab and pertuzumab.

In one aspect, the improved effectiveness of a combination according to the disclosure can be demonstrated by achieving therapeutic synergy. The term “therapeutic synergy” or “synergistic effect” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on parameter tumor volume.

In some embodiments, the patient or subject previously failed at least one line of therapy or standard care of a bone cancer (e.g., osteosarcoma). Non-limiting examples of the failed therapies or standard care of a bone cancer (e.g., osteosarcoma) is any therapy or agent described elsewhere in the present disclosure which can be co-administered with the anti-Cx43 antibodies.

In some embodiments, the patient or subject previously failed at least one therapy for osteosarcoma. In some specific embodiments, the at least one therapy for osteosarcoma comprising chemotherapy, surgery, radiotherapy, or immune therapy, or the combination thereof.

In some specific embodiments, the patient or subject previously failed at least one therapy comprising methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

The patient or subject, before, during, or after being treated with the anti-Cx43 antibody, is examined for cancer related parameters using technologies and methods known in the art. Non-limiting examples of the common medical technologies and methods used to examine and diagnose osteosarcoma include: X-rays, computed tomography (CT) scan, magnetic resonance imaging (MRI), positron emission tomography (PET), bone scintigraphy (Bone Scan), biopsy, EQ-5D questionnaire for assessing quality of life, PERCIST criteria for assessing disease progression, numeric rating scale for assessing pain degree. The choice of technologies and methods, and the frequency of examination can be determined and/or adjusted by a person skilled in the art based on the patient's or subject's specific conditions.

In some embodiments, treatment with the anti-Cx43 antibody for osteosarcoma improves disease progression as assessed by PERCIST criteria, RECIST criteria, and/or ICDS criteria comparing with a control, which can include but is not limited to an osteosarcoma population who receive or failed the same lines of therapy as the subject, but do not receive the anti-Cx43 antibody for osteosarcoma.

In some embodiments, treatment with the anti-Cx43 antibody for osteosarcoma improves overall survival by 1 month, at least 2 months, at least 3 months, at least 4 months, at least 5 months, or at least 6 months comparing with a control value, which can include but is not limited to the mean or median of overall survival of an osteosarcoma population who receive or failed the same lines of therapy as the subject, but do not receive the anti-Cx43 antibody for osteosarcoma.

In some embodiments, treatment with the anti-Cx43 antibody for osteosarcoma improves 6-month progression-free survival by 1 month, at least 2 months, at least 3 months, at least 4 months, at least 5 months, or at least 6 months comparing with a control value, which can include but is not limited to the mean or median of overall survival of an osteosarcoma population who receive or failed the same lines of therapy as the subject, but do not receive the anti-Cx43 antibody for osteosarcoma.

In some embodiments, the subject treated with the anti-Cx43 antibody for osteosarcoma has a duration of response of at least 10 days, at least 15 days, at least 20 days, at least 25 days, at least 1 month, at least 2 months, at least 3 months, at least 4 months, at least 5 months, at least 6 months, or more.

In some embodiments, the subject treated with the anti-Cx43 antibody for osteosarcoma has a time to response less than 2 years, less than 11 months, less than 10 months, less than 9 months, less than 8 months, less than 7 months, less than 6 months, less than 5 months, less than 4 months, less than 3 months, less than 2 months, less than 1 months, less than 25 days, less than 20 days, less than 15 days, less than 10 days, or less than 5 days.

In some embodiments, treatment with the anti-Cx43 antibody for osteosarcoma reduces the tumor size by at least 1%, at least 2%, at least 3%, at least 5%, at least 8%, at least 10%, at least 15%, at least 20%, or at least 30%.

In some embodiments, treatment with the anti-Cx43 antibody for osteosarcoma improves the Europe Health-related Quality of Life Questionnaire 5 Dimensions (EQ-5D) score. In some embodiments, treatment with the anti-Cx43 antibody for osteosarcoma improves the EQ-5D health index to at least 0.1, at least 0.2, at least 0.3, at least 0.4, at least 0.5, at least 0.6, at least 0.7, at least 0.8, at least 0.9, or 1, compared with baseline. In some embodiments, treatment with the anti-Cx43 antibody for osteosarcoma improves the visual analog scale (VAS) score of the EQ-5D questionnaire by at least 1, at least 2, at least 3, at least 4, at least 5, at least 10, at least 20, at least 30, at least 40, at least 50, at least 60, at least 70, at least 80, at least 90, or 100.

In some embodiments, treatment with the anti-Cx43 antibody for osteosarcoma reduces Numeric Rating Scale (NRS) score by at least 1, at least 2, at least 3, at least 4, at least 5, at least 6, at least 7, at least 8, at least 9, or 10.

In some embodiments, treatment with the anti-Cx43 antibody for osteosarcoma improves bone mineral density (BMD) of the lumbar spine, hip, and femur and neck. In some embodiments, treatment with the anti-Cx43 antibody for osteosarcoma improves bone mineral density (BMD) of the lumbar spine, hip, and femur and neck by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

Treatment with the anti-Cx43 antibody for osteosarcoma reduces the dose and/or frequency of morphine use by the subject or patient. In some embodiment, treatment with the anti-Cx43 antibody for osteosarcoma reduces the dose and/or frequency of morphine use by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In certain embodiments, treatment with the anti-Cx43 antibody for osteosarcoma completely tapers the use of morphine use.

As used herein, a “response” or “being responsive” to a treatment refers to the subject having improvement of at least one parameter of disease progression. The subject can have “partial response” or “complete response” to a treatment. The response, partial response or complete response to a treatment can be determined based on methods known in the art. A person skilled in the art can determine the proper methods based on the type of diseases being evaluated. Non-limiting examples of the methods include RECIST criteria, ICDS criteria and PERCIST criteria. In some embodiments, a subject has complete response to a treatment of the anti-Cx43 antibody. In some other embodiments, the subject has a partial response to a treatment of the anti-Cx43 antibody.

In some embodiments, after treatment with the anti-Cx43 antibody, the subject does not experience: (i) adverse event, serious adverse event or grade 3 or higher adverse events; (ii) treatment emergent adverse event (TEAE); (iii) adverse drug reaction (ADR); (iv) Cx43-related cardiac events or severe hepatic events; and/or (v) dose limiting toxicity.

“Adverse Event” or “AE” refers to any adverse medical event in a participant administered with a drug, which may be manifested as symptoms, signs, diseases, or abnormal laboratory tests, but not necessarily causally related to the drug.

“Adverse Drug Reaction” or “ADR” refers to any reaction harmful to human body or unintended reaction which is considered to be possibly related to the drug in a clinical study. The drug and the AE are at least in one possibly reasonable causality, i.e., the correlation cannot be ruled out.

“Treatment Emergent Adverse Event” or “TEAE” refers to the event that occurs during the treatment but does not occur or deteriorates relative to the pre-treatment period.

“Serious Adverse Event” or “SAE” refers to one of the following situations after the participant receives the drug: (i) resulting in death; (ii) Life-threatening: refers to the participant's risk of death at the time of the event, not to the risk of death if the event deteriorates; (3) requiring hospitalization or prolongation of hospitalization. It is understood by a person in the art that SAE includes but is not limited to the foregoing list.

Non-limiting examples of “dose limiting toxicity” include Grade 4 neutropenia lasting for ≥7 days; or Grade ≥3 neutropenia with fever (ANC <1000/mm³ with single temperature ≥38.3° C. or persistent temperature ≥38° C. for more than 1 h) or with infection; Grade 4 thrombocytopenia or Grade 3 thrombocytopenia with hemorrhage; Grade ≥3 hepatotoxicity: ALT or AST ≥5×upper limit of normal (ULN) (for participants with normal baseline) or ≥5×baseline value (for participants with combined liver metastasis and abnormal baseline); or bilirubin ≥3×ULN (for participants with normal baseline value) or ≥3×baseline value (for participants with abnormal baseline value); Grade ≥3 nephrotoxicity: Serum creatinine ≥3×ULN (for participants with normal baseline value) or ≥3×baseline value (for participants with abnormal baseline value); Grade ≥3 cardiotoxicity: QTc ≥500 ms or QTc increase ≥60 ms.

### Examples

The following examples are included to demonstrate preferred embodiments of the invention. It should be appreciated by those of skill in the art that the techniques disclosed in the examples which follow-represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Overview and Preclinical Study of ALMB-0168

ALMB-0168 is a humanized monoclonal antibody prepared from a recombinant CHO cell strain under GMP conditions. Similar to typical humanized antibodies, ALMB-0168 consists of 2 identical heavy chains (IgG4 variants) and 2 identical light chains (κchains) covalently linked by 4 pairs of disulfide bonds. The heavy and light chains consist of 446 and 219 amino acid residues, respectively. Based on the total amino acid composition, the molecular weight is 146.2 kDa. Similar to most antibodies, the heavy chains are glycosylated at the conserved glycosylation site in the constant region.

ALMB-0168 is a novel Cx43 humanized IgG4 monoclonal antibody targeting the Cx43 hemichannels of osteocytes. Initial antibodies are obtained by immunizing mice with human Cx43. Mouse antibodies are then humanized using a common humanization approach, i.e., complementary determining region (CDR) transplantation (Bowers et al., J Biol Chem., 288(11): 7688-7696 (2013)) (also known as modification) and germline sequence modeling.

ALMB-0168 is the first drug designed to activate the Cx43 hemichannels and inhibit the growth of primary and metastatic bone tumors by activating Cx43 hemichannels to release critical substances such as ATP into the extracellular environment.

Based on the preclinical evaluation, ALMB-0168 can effectively activate osteocyte Cx43 hemichannel opening both in vivo and in vitro. In several mouse models of breast cancer bone metastasis and in situ osteosarcoma, administration of ALMB-0168 inhibited tumor growth in a dose-dependent manner and was able to prolong the lifespan of tumor-bearing animals, suggesting its potential as a treatment for malignant bone tumors. In addition, ALMB-0168 has good nonclinical safety attributes that support the FIH dose. The physicochemical properties of ALMB-0168 are shown in Table 1.

TABLE 1
Physicochemical Properties of ALMB-0168

| Categories | Characteristics |
|---|---|
| Subtype of immunoglobulin | IgG4 |
| Appearance | Colorless or pale yellow clear liquid, |
| | with slight opalescence |
| Solubility | 25 mg/ml in formulation buffer (pH 5.5) |
| Isoelectric Point (pI) | 8.1 |
| Theoretical Extinction | 1.531 |
| Coefficient | |

### Pharmacological Action and Action Mechanism

Osteocytes have high expression of connexin Cx43 in interstitial junction channels and hemichannels. The gap junction channels mediate cellular communication between 2 adjacent cells to maintain the normal metabolic function of the cells, while the hemichannels allow molecular exchange between the cells and the extracellular environment. Hemichannels are normally closed, but under certain conditions, such as mechanical stress or ischemia, cytokine or ion stimulation, they will be activated and opened. Opened hemichannels allow small molecules (<1.2 kDa) such as ATP, NAD+, glutamic acid, and inositol triphosphate (IP3) to pass (Goodenough et al., Nature reviews Molecular cell biology, 4:285-294 (2003)). It has been confirmed that the opening of the Cx43 hemichannels of osteocytes has a strong inhibitory effect on the bone metastasis of breast cancer tumors (Zhou et al., Oncogene, 35: 5597-5607 (2016)).

ALMB-0168 is a humanized IgG4 monoclonal antibody that targets the osteocyte hemichannels. Initial antibodies are obtained by immunizing mice with human Cx43. Mouse antibodies are then humanized using a common humanization approach, i.e., complementary determining region (CDR) transplantation (Bowers et al., J Biol Chem., 288(11): 7688-7696 (2013)) (also known as modification) and germline sequence modeling.

ALMB-0168 is designed to activate the Cx43 hemichannels, and allows critical anti-cancer agents (e.g., ATP) to release into the extracellular environment by activating Cx43 hemichannels. On the one hand, ATP can activate killer CD8+ T cells and CD4+ T cells, while significantly inhibiting Treg cells with negative tumor immune conditions; on the other hand, ATP can bind to P2Y11 receptors on cancer cells and reduce the positive regulation of P2Y11 on CXCR4, thus inhibiting the growth and metastasis of primary and metastatic bone tumors.

Based on the preclinical evaluation, ALMB-0168 can effectively activate osteocyte Cx43 hemichannel opening both in vivo and in vitro. In several mouse models of breast cancer bone metastasis and in situ osteosarcoma, ALMB-0168 inhibited tumor growth in a dose-dependent manner and was able to prolong the lifespan of tumor-bearing animals, suggesting its potential as a treatment for malignant bone tumors. In addition, ALMB-0168 has good nonclinical safety attributes that support the FIH.

### Overview of Preclinical Study

### Overview of Preclinical Pharmacodynamic Study

### In Vitro Test

In vitro test showed ALMB-0168 had no effect on gap junction. ALMB-0168 did not affect the normal function of gap junction. After treatment of murine bone MOL-Y4 cells with ALMB-0168 for 16 h, there was no effect on gap junction, while the positive control glycyrrhetinic acid (known gap junction blocker) significantly inhibited gap junction function (p <0.001).

ALMB-0168 specifically bound and activated the Cx43 hemichannels of osteocytes. The binding activity of ALMB-0168 to mouse osteocytes (MOL-Y4) and primary human osteocytes was determined by ELISA (Enzyme-Linked Immunosorbent Assay). The results showed that the antibody concentrations corresponding to the concentration for 50% of maximal effect (EC50) for MOL-Y4 cells and human osteocytes were 37.3±0.6 μg/ml and 41.0±6.5 μg/ml, respectively. In addition, binding of ALMB-0168 to its target effectively activated the Cx43 hemichannel opening, and EC50 values were reached at 2.33±0.15 μg/ml and 3.97±0.90 μg/ml for MOL-Y4 cells and human osteocytes, respectively, as determined by EB staining.

ALMB-0168 treatment inhibited migration of breast cancer and osteosarcoma cells. Conditioned medium harvested from ALMB-0168-treated osteocytes (MOL-Y4) significantly inhibited the migration of breast cancer cells (MDA-MB-231) and osteosarcoma cells (OS17) in vitro (p<0.001). Cell migration was determined by a 24-well tissue culture plate migration test inserted with a membrane filter. The results showed that ALMB-0168-treated conditioned medium of osteocytes inhibited cancer cell migration by 50% compared with vehicle-treated conditioned medium of osteocytes. The inhibitory effect disappeared after the conditioned medium was treated with ATPase (an enzyme for hydrolyzing ATP), indicating that the extracellular ATP level was increased after treatment with ALMB-0168.

### In Vivo Test

Tissue distribution and activation of Cx43 hemichannels of ALMB 0168 in mice. At 4 h after intraperitoneal injection of ALMB-0168 (25 mg/kg), antibodies were only detected in mouse osteocytes, but not in other organs expressing connexins (liver, heart and spleen). In addition, ALMB-0168 treatment significantly increased the Cx43 hemichannel opening of mouse osteocytes as shown by EB staining assay.

ALMB 0168 inhibited the bone metastasis of breast cancer in mice. Once-weekly injection of ALMB-0168 at 25 mg/kg significantly inhibited human breast cancer growth in a mouse xenograft model (p<0.05).

ALMB-0168 inhibited osteosarcoma growth and prolonged tumor-bearing mouse survival in a mouse in situ transplantation model. In a mouse model of in situ osteosarcoma, once-weekly administration of 15-25 mg/kg ALMB-0168 significantly inhibited tumor growth in the tibia and prolonged life span.

ALMB-0168 treatment increased ATP levels in plasma. To assess the change in plasma ATP levels due to activation of the Cx43 hemichannels by ALMB-0168 administration, ATP levels in plasma were measured by luciferin/luciferase assay following injection of ALMB-0168 in mice. The results showed that ALMB-0168-treated mice had significantly (p<0.001) higher ATP levels than saline-treated mice by more than 5-fold.

### Overview of Preclinical Pharmacokinetics Study

Pharmacokinetic Study of ALMB-0168 in Cynomolgus Monkeys after Single Intravenous Infusion

A total of 18 cynomolgus monkeys (9/sex) were randomized into 3 dose groups: low-, medium- and high-dose groups, with IV doses of 5, 15 and 50 mg/kg, respectively. Blood samples were collected from each group pre-dose, at the end of dosing, at 10 min post-dose, and at 2 h, 8 h, 24 h, 48 h, 72 h, 5 d, 7 d, 10 d, 14 d, 17 d, 21 d, and 28 d post-dose. Concentrations of ALMB-0168 in serum were determined using a validated ELISA method with a lower limit of quantification of 0.01 μg/ml. PK parameters were determined using non-compartmental analysis (NCA) in WinNonlin software.

### Serum PK Parameters are Summarized as Follows:

1. There were no significant sex differences in PK parameters (p-value ≥0.05), except that the AUC was slightly higher in females than in males in the medium-dose group (p-value <0.05, ratio=1.32).

2. Following a single intravenous infusion of ALMB-0168 in cynomolgus monkeys, the maximum serum concentration and systemic exposure of ALMB-0168 were dose proportional. The dosing ratio in the low-, medium- and high-dose groups was 1:3:10. The mean Cmax and AUClast ratios in the low-, medium-, and high-dose groups were 1:3.04:10.92 and 1:3.10:10.95, respectively.

3. Regression analysis was performed on Cmax- dose and AUClast- dose. The results showed that the slopes of the Cmax- dose and AUClast- dose curves were 1.04 and 1.04, respectively, the 95% confidence intervals were [1.11, 0.98] and [1.15, 0.94], respectively, and the R2 was 0.99 and 0.96, respectively. The results showed that the exposure of ALMB-0168 in cynomolgus monkeys was linearly related to the single intravenous infusion doses of 5, 15 and 50 mg/kg.

### Overview of Preclinical Toxicology Study

Single-Dose Toxicity Study ofALMB-0168

120 (60/sex) were randomized to Groups 1, 2, 3 and 4, with 15 mice/sex in each group. Mice in Group 1 (0 mg/kg) were treated with ALMB-0168 buffer as the vehicle control, and mice in Groups 2, 3 and 4 were treated with ALMB-0168 at 50 mg/kg, 150 mg/kg and 500 mg/kg, respectively.

Under the conditions of the study, no significant test article-related toxicity was observed in mice receiving a single IV dose of ALMB-0168 at doses of 50, 150, and 500 mg/kg.

A total of 8 cynomolgus monkeys (4/sex) were randomized to Groups 1-4, with 1/sex in each group. Placebo treatment was used as a control in Group 1, and ALMB-0168 50, 150 or 500 mg/kg was dosed to animals in Groups 2, 3 and 4, respectively. On Day 1, cynomolgus monkeys received a single IV infusion. Animals were observed for approximately 4 h after dosing, followed by regular clinical observations. Body weight, food consumption and body temperature were measured, and ECG, hematology, coagulation, clinical chemistry, urinalysis, and serum anti-drug antibody and toxicokinetic analysis were performed. On Day 15, all animals were sacrificed and a full macroscopic observation was performed.

No mortality or morbidity was observed in any of the animals in Groups 1-4 during the study. There were no test article-related abnormal clinical findings in any of the animals.

### Multiple-Dose Toxicity Study of ALMB-0168

### 1. Repeat-Dose Toxicity Study in Monkey

Forty-eight cynomolgus monkeys were randomized to Groups 1-4, 24/sex, and 6/sex/group. ALMB-0168 buffer was used as the vehicle control in Group 1, and 25, 75 and 250 mg/kg ALMB-0168 were used in Groups 2, 3 and 4, respectively. Cynomolgus monkeys were dosed once weekly for 6 consecutive weeks on Days 1, 8, 15, 22, 29, 36, and 43 (7 doses in total).

No signs of death or moribundity were observed in animals in Groups 1-4 throughout the study.

Throughout the study, there were no analyte-related or toxicologically significant abnormal changes in body weight, food consumption, body temperature, cardiovascular function, respiratory function, ECG, blood pressure, ophthalmology, hematology, coagulation, clinical chemistry, urinalysis, lymphocyte subsets, cytokines, serum immunoglobulins, serum complements and functional observation battery to assess neurobehavior in animals treated with 25, 75 and 250 mg/kg ALMB-0168 compared with animals of the same sex in the vehicle control group.

Serum anti-drug antibody (ADA) analysis: In the 25 mg/kg ALMB-0168 dose group, 2/12 monkeys tested positive for ADA on Day 15 post-dose, with the highest titer of 1:4. In the 75 mg/kg AMLB-0168 dose group, 1/12 monkeys tested positive for ADA on Day −2 pre-dose and Day 15 post-dose, with titers less than 1; no increase in titers was observed throughout the study. Therefore, the positive result was considered unrelated to ALMB-0168. In the 250 mg/kg ALMB-0168 dose group, 2/12 monkeys tested positive for ADA on Day 15 post-dose with the highest titer of 1:8. The TK results showed that the Cₘₐₓ and AUCₗₐₛₜ of ADA-positive animals in the same group were not significantly different from those of other animals.

Toxicokinetics: The mean TK parameters of the test article in cynomolgus monkey serum are shown in Table 2. All reported values were mean except Tₘₐₓ, which was reported as median.

TABLE 2
Mean TK Parameters of ALMB-0168 in Cynomolgus
Monkey Serum (Repeated Dose Study)

| | Group | Sex | | t_{1/2} | Tₘₐₓ | Cₘₐₓ | AUC_{0-168 h} | AUC_{inf} | |
|---|---|---|---|---|---|---|---|---|---|
| Cycle | (mg/kg) | (n = 6/sex) | | h | h | μg/ml | h · mg/ml | h · mg/ml | |
| D1 | Low | Male | Mean | 175 | 2 | 735 | 64.2 | 130 | |
| | dose | | SD | 56.6 | 0.00 | 47.2 | 4.09 | 29.4 | |
| | 25 | Female | Mean | 182 | 0.33 | 729 | 63.5 | 131 | |
| | | | SD | 56.9 | 0.00 | 52.5 | 4.60 | 26.9 | |
| | | p-value | | 0.84 | 0.74 | 0.85 | 0.80 | 0.80 | |
| | Medium | Male | Mean | 169 | 2 | 2220 | 195 | 378 | |
| | dose | | SD | 32.6 | 0.84 | 265 | 11.8 | 51.4 | |
| | 75 | Female | Mean | 174 | 2 | 2170 | 183 | 362 | |
| | | | SD | 30.0 | 0.00 | 236 | 21.0 | 30.3 | |
| | | p-value | | 0.80 | 0.43 | 0.71 | 0.26 | 0.52 | |
| | High | Male | Mean | 149 | 2 | 7660 | 591 | 1050 | |
| | dose | | SD | 29.3 | 0.00 | 683 | 37.2 | 121 | |
| | 250 | Female | Mean | 131 | 2 | 7690 | 580 | 961 | |
| | | | SD | 40.2 | 0.00 | 365 | 55.9 | 222 | |
| | | p-value | | 0.39 | 1.00 | 0.93 | 0.70 | 0.39 | |
| D22 | Low | Male | Mean | 193 | 0.33 | 1300 | 136 | 302 | 2.11 |
| | dose | | SD | 46.9 | 0.00 | 151 | 15.1 | 75 | 0.16 |
| | 25 | Female | Mean | 190 | 2 | 1040 | 100 | 228 | 1.58 |
| | | | SD | 67.5 | 0.84 | 173 | 22.1 | 90.6 | 0.34 |
| | | p-value | | 0.93 | 0.10 | 0.02 | 0.01 | 0.02 | |
| | Medium | Male | Mean | 145 | 0.33 | 3670 | 356 | 649 | 1.82 |
| | dose | | SD | 38.6 | 0.00 | 753 | 56.8 | 152 | 0.23 |
| | 75 | Female | Mean | 170 | 1.165 | 3510 | 314 | 622 | 1.73 |
| | | | SD | 40.1 | 0.84 | 304 | 23 | 111 | 0.19 |
| | | p-value | | 0.30 | 0.29 | 0.63 | 0.13 | 0.74 | |
| | High | Male | Mean | 152 | 2 | 11300 | 1100 | 2030 | 1.87 |
| | dose | | SD | 23.6 | 0.00 | 841 | 90.2 | 150 | 0.18 |
| | 250 | Female | Mean | 149 | 2 | 10800 | 956 | 1790 | 1.64 |
| | | | SD | 45.9 | 0.00 | 409 | 137 | 564 | 0.10 |
| | | p-value | | 0.89 | 0.34 | 0.22 | 0.06 | 0.34 | |
| D43 | Low | Male | Mean | 239 | 0.33 | 1220 | 132 | 343 | 2.11 |
| | dose | | SD | 53.0 | 0.00 | 104 | 14.4 | 105 | 0.13 |
| | 25 | Female | Mean | 223 | 0.33 | 1290 | 121 | 296 | 1.83 |
| | | | SD | 62.2 | 0.00 | 205 | 21.8 | 94.8 | 0.38 |
| | | p-value | | 0.81 | 0.20 | 0.46 | 0.63 | 0.68 | |
| | Medium | Male | Mean | 166 | 2 | 3310 | 363 | 751 | 1.81 |
| | dose | | SD | 19.8 | 0.84 | 384 | 52.4 | 165 | 0.11 |
| | 75 | Female | Mean | 169 | 0.33 | 3280 | 329 | 670 | 2.02 |
| | | | SD | 67.9 | 0.00 | 258 | 18.9 | 206 | 0.50 |
| | | p-value | | 0.96 | 0.05 | 0.88 | 0.48 | 0.71 | |
| | High | Male | Mean | 166 | 2 | 11000 | 1100 | 2050 | 1.82 |
| | dose | | SD | 19.8 | 0.00 | 618 | 22.9 | 219 | 0.18 |
| | 250 | Female | Mean | 169 | 1.165 | 9440 | 882 | 1540 | 1.47 |
| | | | SD | 67.9 | 0.84 | 505 | 113 | 481 | 0.01 |
| | | p-value | | 0.96 | 0.05 | 0.00 | 0.12 | 0.31 | |

Abbreviations:
AF = Accumulation factor (AUC_{Day22}/AUC_{Day1});
AUC = Area under the concentration-time curve;
Cₘₐₓ = Maximum concentration;
N = Number of animals per sex per group;
Tₘₐₓ = Time to maximum concentration;
t_{1/2} = Half-life;
SD = Standard deviation

Under the conditions of the study, ALMB-0168 was administered intravenously to cynomolgus monkeys at 25, 75, and 250 mg/kg once weekly for 6 consecutive weeks (7 doses in total), followed by a four-week recovery period. Test article-related local injection site reactions were observed, and there was a dose-dependent decrease in the number of lymphocytes in the spleen of animals in all dose groups. After 4 weeks of recovery, the chronic active and hemorrhagic injection site inflammations were completely recovered in all animals except 1 animal in the low-dose group. The findings observed in the spleen (dose-dependent decrease in the number of lymphocytes in the germinal center and border zone) recovered completely in the and 75 mg/kg dose groups after the 4-week recovery period, with a tendency to recover in the 250 mg/kg dose group (minimal in 1 male and mild in 1 female). Therefore, this finding was considered to have no adverse effects on the spleen. Therefore, 250 mg/kg was the no-observed-adverse-effect level (NOAEL) in the study.

### 2. Repeat-Dose Toxicity Study in Mice

980 mice (490/sex) were randomized into 8 groups (Groups 1-8), with 40 mice/sex/group in Groups 1, 2, 3 and 4, 45 mice/sex/group in Group 5, and 95 mice/sex/group in Groups 6, 7 and 8. Mice were administered intravenously once a week for 6 consecutive weeks (7 doses in total) at the following doses: Groups 1 and 5 (vehicle control groups) (ALMB-0168 buffer, 0 mg/kg), ALMB-0168 25 mg/kg (Groups 2 and 6), 75 mg/kg (Groups 3 and 7), and 250 mg/kg (Groups 4 and 8). The day of administration was defined as Day 1.

Parameter indicators evaluated in the study included clinical observation, body weight, food consumption, fundus examination, hematology, coagulation, clinical chemistry, urinalysis, T lymphocyte subset determination (CD3+, CD3+CD4+, CD3+CD8+, CD4+CD8+), and cytokine test (IL-2, IL-4, IL-5, IL-6, TNF-α, IFN-γ). Six weeks after administration (Day 44), the first 20 animals/sex/group in Groups 1, 2, 3, and 4 were euthanized and the remaining 20 animals/sex/group were euthanized after the next 4-week recovery period (Day 72). Gross anatomy was performed on all animals in Groups 1 to 4, organ weights were collected, and gross anatomical observations and microscopic examination were completed. Histopathological evaluations were performed on all standard tissues in Groups 1 and 4, tissues of animals that died unexpectedly before the scheduled time point, and all abnormal tissues in Groups 2 and 3.

There were no test article-related deaths or moribundities in any of the animals in Groups 1-8 throughout the study.

No test article-related abnormal changes were observed in clinical observations and injection site observations throughout the study.

Test article-related abnormal changes in body weight, body weight gain, food consumption, body temperature, ophthalmic examination, hematology, coagulation, T lymphocyte subsets, and cytokines were not observed in animals dosed with 25, 75, and 250 mg/kg ALMB-0168 throughout the study.

Animals dosed with 25, 75 and 250 mg/kg ALMB-0168 (except females dosed with 25 mg/kg) had increased LDH on Day 44. After the 4-week recovery period, LDH decreased to the level of the vehicle control group. There were no microscopic findings. So it was not considered an adverse reaction.

There were no test article-related abnormalities in organ weights, organ/body weight ratio, organ/brain weight ratio, gross anatomical observations, and microscopic examinations in any of the animals at the end of dosing and at the end of the 4-week recovery period. No test article-related local irritation was observed at the injection site in any of the animals in the study.

In conclusion, under the conditions of the study, mice were administered ALMB- 0168 at 25, 75, and 250 mg/kg by intravenous injection once a week for 6 consecutive weeks (7 doses in total), followed by a 4-week recovery period. The results showed that no adverse systemic toxicity and target organ toxicity were observed. Therefore, the NOAEL for ALMB-0168 in the study was 250 mg/kg.

### Other Related Toxicity Studies

### 1. Tissue Cross-reaction Study in Normal Cynomolgus Monkey Tissues

Tissue cross-reactivity of ALMB-0168 was evaluated in 34 types of different normal cynomolgus monkey tissues (each type of tissue was from three different animals) using streptavidin-biotin immunohistochemistry.

Positive staining was observed in the cell membranes of positive control section MLO-Y4 cells, a mouse osteocyte line highly expressing Cx43 hemichannels, following staining of the test article with 5 μg/ml and 20 μg/ml ALMB-0168-biotin, respectively; negative control section of the test article group showed no positive staining on the frozen tissue of human skeletal muscle. No positive staining was observed in the positive control photos and negative control photos of the isotype control group and negative control group.

In the test article group (ALMB-0168-biotin, 5 μg/ml and 20 μg/ml), non-specific staining of round cells was observed in bone marrow, ileum and stomach tissues of the isotype control group and negative control group, and non-specific staining was also observed for renal tubular epithelial cells and hepatocytes. No non-specific positive staining was observed in other tissues of each test group.

Under the conditions of the study, no specific staining was observed for ALMB-0168 in normal cynomolgus monkey tissues.

### 2. Tissue Cross-Reaction Test in Normal Human Tissues

The streptavidin-biotin method of IHC staining was used to evaluate whether ALMB-0168 had a tissue cross-reactivity with 34 frozen tissues from normal human (each tissue derived from 3 different individuals).

In the test article group (ALMB-0168-biotin, 5 μg/ml and 20 μg/ml), non-specific staining of round cells was observed in bone marrow, colon, ileum and stomach tissues of the isotype control group and negative control group, and non-specific staining was also observed for renal tubular epithelial cells and hepatocytes. No non-specific positive staining was observed in other tissues of each test group.

Under the conditions of the test, no specific staining was observed for ALMB-0168 in frozen normal human tissues.

### 3. Tissue Reaction Test in Mouse Tissues

The streptavidin-biotin method of IHC staining was used to evaluate whether ALMB-0168 had a cross reactivity with 30 frozen tissues from normal mice (each tissue derived from 3 different individuals).

In the isotype control group and negative control group of the test article group (ALMB-0168-biotin, 5 μg/ml and 20 μg/ml), non-specific staining was observed in cortical cells and bone marrow cells of the adrenal gland, round cells of the stomach, and epithelial cells and hepatocytes of the renal tubule. No non-specific positive staining was observed in other tissues of each test group.

Under the conditions of the test, no specific staining was observed for ALMB-0168 in normal mouse tissues.

### 4. In Vitro Hemolysis Test on Human Blood Cells

provide a reference for the clinical study of the test article. The test article at a concentration of 10 mg/ml was analyzed on the day of the in vitro test. The accuracy of the upper, middle and lower layers of the test article was 99.93%, 101.20% and 101.63%, respectively, and the coefficient of variation was 0.87%. The test article was not detected in the negative control and positive control. The analytical results met the requirements.

After incubation at 37±0.5° C. for 3 h, the positive control tube was completely hemolyzed, the solution was clear red, and there was no RBC at the bottom of the tube. For the test article tube and negative control tube, erythrocytes were observed to sink to the bottom of the tube, and the upper solution was colorless and clear, without hemolysis and aggregation.

Under the conditions of this study, ALMB-0168 at the concentration of 10 mg/ml had no hemolytic effect on human RBCs in vitro and did not cause RBC aggregation.

### 5. Cytokine Release Test in Human Peripheral Blood Mononuclear Cells (PBMCs)

In vitro cytokine release from stimulated PBMC samples was tested after 24-hour incubation of the PBMC samples with ALMB-0168. For technical control, study-grade anti-CD3 antibody and study-grade human IgG isotype control were used as positive and negative controls, respectively, while the untreated PBMC sample was used as baseline control.

All donor-derived PBMCs reacted with CD3 antibodies in both stimulation forms, indicating that the PBMC samples had the ability to stimulate and induce the release of IL-2, IL-6, IL-10, IFN-γ and TNF-α. Elevated IL-6 was detected in some PBMC samples, and wet coating stimulation was used for most of these samples.

Human IgG controls in both stimulation forms hardly induce the release response of IL-2, IL-10, IFN-γ, and TNF-α. Under the soluble stimulation form, the IL-6 responses of multiple donors were different. No dose-response trend was observed, and the response did not reach the extent of positive control biological CD3 antibodies reached by the majority of donors.

Under both forms of stimulation, ALMB-0168 induced little response to IL-2, IL-10, IFN-γ and TNF-α cytokine release. Under the soluble stimulation form, the IL-6 responses of multiple donors were different. The response did not reach the extent of positive control biological CD3 antibodies reached by the majority of donors.

### First in Human Safety Study in Australia

As of May 2021, a total of 2 participants (1 mg/kg in one participant, 3 mg/kg in another participant) had been enrolled in the first-in-human study in Australia and had received 5 cycles of dosing (once every 3 weeks). There were no serious adverse events, adverse events leading to death, or adverse events leading to participant withdrawal. The drug-related adverse event that occurred in 1 participant in the 1 mg/kg group was abdominal pain, which fluctuated between Grade 1 and Grade 2, and recovered after symptomatic treatment with oral drugs. The drug-related adverse event in 1 participant in the 3 mg/kg group was Grade 1 fatigue, which recovered spontaneously without drug treatment. During the study, other adverse events that occurred in both participants included diarrhea, abdominal discomfort, nausea, herpes on the left side of the upper lip, hyperglycemia, hypercalcemia, and redness and itching at the peripherally inserted central catheter site, all of which were Grade 1, and were judged to be unrelated to the drug.

### Known Potential Benefits

Prior to the initiation of the study, a first-in-human clinical study of ALMB-0168-AU-101 has been conducted in Australia to evaluate the safety, tolerability, PK, PD, preliminary efficacy, and immunogenicity of ALMB-0168 in patients with incurable osteosarcoma and bone metastatic tumors with no better treatment option, and guide the starting dose of the study to reduce the exposure to ineffective dose and further ensure safety. For this first-in-human clinical study, the dosing of the first and second participants in the first and second dose group (1 mg/kg and 3 mg/kg, respectively) has been and demonstrated good safety and tolerability. As described in the study protocol, all participants were closely monitored throughout the study and any adverse reactions experienced by the participants were closely monitored and handled in a timely manner until they return to normal or are clinically stable.

In conclusion, ALMB-0168 is a humanized monoclonal IgG4 antibody targeting Cx43 hemichannels, with a clear mechanism of action. Preclinical studies have shown that ALMB-0168 has good efficacy, safety and controllability, and may bring efficacy benefits to patients with osteosarcoma and bone metastatic tumors.

### Example 2. A Multicenter, Single-Arm, Open-Label Phase I/II Clinical Study to Evaluate the Safety and Efficacy of ALMB-0168 in Patients with Osteosarcoma—Study Protocol

This study is registered at ClinicalTrials.gov as NCT04886765.

### Objectives and Endpoints of The Study

### Part I of The Study

The primary objective of Part I is to evaluate the safety and tolerability of ALMB-0168 in patients with osteosarcoma who have failed the standard of care and to explore the recommended Phase II dose (RP2D).

The secondary objectives of Part I are:
to evaluate the pharmacokinetic (PK) profile of ALMB-0168;
to evaluate the efficacy of ALMB-0168 in the treatment of patients with osteosarcoma;
to evaluate the immunogenicity of ALMB-0168.

The primary endpoints of Part I are:
physical examination, vital signs, pulse oxygen saturation, safety laboratory tests, ECG, and occurrence of AEs, SAEs and DLTs;
MTD (if any).

The secondary endpoints of Part I are:
pharmacokinetic indicators: including but not limited to AUC0-t, AUC0-inf, Cmax, Ctrough, Tmax, CL and λz, and t1/2;
efficacy indicators: overall response rate (ORR), progression-free survival (PFS), duration of response (DoR), time to response (TTR), disease control rate (DCR), 6-month progression-free survival rate (6m-PFSR), overall survival (OS); rate of change from baseline in alkaline phosphatase (ALP) or lactate dehydrogenase (LDH); skeletal related events (SREs); change from baseline in Numeric Rating Scale (NRS) and Europe Health-related Quality of Life Questionnaire 5 Dimensions (EQ-5D) scores; bone mineral density of the lumbar spine, hip, and femur and neck; dose and frequency of morphine use;
incidence of anti-drug antibodies (ADA).

### Part II of The Study

The primary objective of Part II is to further evaluate the safety and efficacy of ALMB-0168 in patients with high-grade osteosarcoma who failed after standard of care to further determine RP2D.

The secondary objectives of Part II are:
to further evaluate the pharmacokinetic (PK) profile of ALMB-0168;
to evaluate the efficacy of ALMB-0168;
to further evaluate the immunogenicity of ALMB-0168.

The primary endpoints of Part II are:
physical examination, vital signs, pulse oxygen saturation, safety laboratory tests, ECG, and occurrence of AEs and SAEs;
efficacy indicators: 6-month progression-free survival rate (6m-PFSR).

The secondary endpoints of Part II are:
efficacy indicators: Overall response rate (ORR), duration of response (DoR), time to response (TTR), disease control rate (DCR), overall survival (OS); rate of change from baseline in alkaline phosphatase (ALP) or lactate dehydrogenase (LDH); Skeletal related events (SREs); change from baseline in Numeric Rating Scale (NRS) and Europe Health-related Quality of Life Questionnaire 5 Dimensions (EQ-5D) scores; bone mineral density of the lumbar spine, hip, and femur and neck; dose and frequency of morphine use;
PK indicators: Include but are not limited to Cmax and Ctrough;
incidence of anti-drug antibodies (ADA).

### Study Design

### Overall Design

This is a multicenter, open-label, single-arm Phase I/II clinical study to evaluate the safety and preliminary efficacy of ALMB-0168 in patients with osteosarcoma who have failed the standard of care.

The study will be conducted in two stages (PART I: dose escalation stage and PART II: dose expansion stage), and all participants will receive multiple cycles of intravenous injection of ALMB-0168, once every 3 weeks. PART I study enrolled high-grade osteosarcoma patients who failed the standard of care, and PART II study enrolled high-grade osteosarcoma patients from PART I study population. An accelerated titration and a standard “3+3” escalation design will be used in the dose escalation stage. The escalation method of accelerated titration will be used for the first 2 dose groups (1 mg/kg, 3 mg/kg). One participant will be enrolled in each group, and the dose will be escalated to the next group if the participant does not experience a Grade ≥2 TEAE related to the drug within 3 weeks after the first dose. If the participant experienced a Grade ≥2 TEAE related to the drug, the 3+3 dose escalation method will be used for the dose level. The dose escalation method of 3+3 will be used starting from the 3rd dose group (6 mg/kg). The dose escalation of each subsequent cohort will be judged according to the incidence of ALMB-0168-related adverse events (AEs) evaluated by NCI CTCAE v5.0 within 3 weeks after the first dose (DLT observation period). Dose escalation will continue until ≥1/3 of the participants at the dose level (when ≥6 participants have been assessed) experience a DLT. The tolerable level before the dose level will be considered the MTD, and the safety can be re-assessed by selecting an appropriate dose between the two doses after discussion and communication between the Sponsor and the investigator.

The selection of RP2D from the tolerated dose range will be based on short- and long-term safety information, as well as findings from preclinical dose-finding studies, PK, PD and anti-tumor activity studies. For dose groups that have completed the DLT observation period, after a comprehensive analysis of the participants' benefits by the investigator and the Sponsor based on the assessment of previous clinical data, they can be expanded to a maximum of 8 participants (excluding the MTD expansion cohort) to further provide information on safety, PK profile and anti-tumor activity, and the safety data of the expanded participants will not be used as the basis for determining the MTD.

In the dose expansion stage, an expansion study will be conducted in 1-3 dose groups to explore the initial efficacy in patients with high-grade osteosarcoma who have failed the standard of care, and further describe the safety, PK properties and so on of the ALMB-0168. A maximum of 60 participants are planned to be enrolled in each dose group, and whether to increase participants will be decided after efficacy and safety data are obtained.

All participants will sign the ICF prior to any specific study activity. Eligible participants will be treated at the dose level determined in the dose escalation stage after enrollment. Theoretically, 21 days (3 weeks) is defined as 1 treatment cycle in this study. All participants will receive an intravenous injection of ALMB-0168 every 3 weeks. The participants will receive multiple cycles of treatment until progressive disease or anti-tumor treatment becomes necessary. All participants will be closely monitored for safety.

All participants will be followed in person 28 days after the last treatment, followed by a one-year follow-up to assess long-term outcomes.

### Dose Escalation Design

### Dose-Limiting Toxicity

Dose Limiting Toxicities (DLTs) are defined as the following toxic responses related to the ALMB-0168 that occur during the first treatment cycle (21 days):
Grade 4 neutropenia lasting for ≥7 days; or Grade ≥3 neutropenia with fever (ANC <1000/mm³ with single temperature ≥38.3° C. or persistent temperature ≥38° C. for more than 1 h) or with infection;
Grade 4 thrombocytopenia or Grade 3 thrombocytopenia with hemorrhage;
Grade ≥3 hepatotoxicity: ALT or AST ≥5×upper limit of normal (ULN) (for participants with normal baseline) or ≥5×baseline value (for participants with combined liver metastasis and abnormal baseline); or bilirubin ≥3×ULN (for participants with normal baseline value) or ≥3×baseline value (for participants with abnormal baseline value);
Grade ≥3 nephrotoxicity: Serum creatinine ≥3×ULN (for participants with normal baseline value) or ≥3×baseline value (for participants with abnormal baseline value);
Grade ≥3 cardiotoxicity: QTc ≥500 ms or QTc increase ≥60 ms;
Other Grade ≥3 non-hematological toxicities, but excluding:
   Grade 3 nausea and/or vomiting, lasting less than 72 h in the absence of prophylactic and effective antiemetic treatment;
   Fatigue or asthenia lasting less than 7 days;
   Transient and asymptomatic laboratory test abnormalities lasting less than 3 days;
   Alopecia, rash and diarrhea that are controllable under standard supportive treatment.
   Toxic responses for which the investigator judges that the ALMB-0168 should be permanently discontinued.

The above AEs will be graded according to the National Cancer Institute-Common Terminology Criteria for Adverse Events (NCI-CTCAE) Version 5.0.

Maximum Tolerated Dose

Definition of MTD: An accelerated titration and a standard “3+3” escalation design will be used in the dose escalation stage: The escalation method of accelerated titration will be used for the first 2 dose groups (1 mg/kg, 3 mg/kg). One participant will be enrolled in each group, and the dose will be escalated to the next dose group if the participant has no Grade ≥2 toxicity related to the ALMB-0168. If the participant experienced a Grade ≥2 toxicity related to the ALMB-0168, the 3+3 dose escalation method will be used for the dose level. The dose escalation method of 3+3 will be used starting from the 3rd dose group (6 mg/kg). The dose escalation of each subsequent cohort will be judged according to the incidence of ALMB-0168-related adverse events (AEs) evaluated by NCI CTCAE v5.0 within 3 weeks after the first dose (DLT observation period). Dose escalation will continue until ≥1/3 of the participants experience DLT (when ≥6 participants are assessed) at the dose level. The tolerable dose level before the dose level will be considered the MTD.

After cycle 1, all Grade ≥4 adverse events during the extension period will also be one of the factors in determining dose escalation and MTD.

### Estimation of Initial Dose

The results of toxicity studies in non-human primates and mice will be used to calculate the safe clinical starting dose for this study. The specific method is consistent with the guidance provided in ICH S9. The no-observed-adverse-effect level (NOAEL) of ALMB-0168 is 250 mg/kg in both non-human primates and rodents. Considering the novel target of ALMB-0168, a safety factor exceeding 100×NOAEL will be used to ensure that the first dose will not cause adverse effects in humans. The final starting dose of the drug in this first-in-human study is 1 mg/kg.

### Determination of Maximum Escalation Dose

In the non-GLP dose range-finding study in non-human primates (NIP), ALMB-0168 was administered intravenously to cynomolgus monkeys at doses of 20, 60, and 200 mg/kg weekly, and no adverse reactions were observed in any dose group in the following 2 weeks. Subsequently, in a GLP repeat-dose toxicology study, cynomolgus monkeys were administered intravenously at doses of 25, 75, and 250 mg/kg once weekly for 6 weeks, followed by a 4-week recovery period. No significant adverse reaction were found. In addition, in the GLP repeated-dose toxicology study in mice, mice were repeatedly dosed at 25, 75 and 250 mg/kg weekly for 6 weeks, and no significant adverse reactions were observed after 4 weeks of observation and recovery. Therefore, the NOAEL of ALMB-0168 is 250 mg/kg in both NHP and rodents.

In the preclinical efficacy study, injection of ALMB-0168 at 25 mg/kg once a week significantly inhibited human breast cancer growth in a mouse xenograft model. In a mouse model of in situ osteosarcoma, once-weekly administration of 15-25 mg/kg ALMB-0168 significantly inhibited tumor growth in the tibia and prolonged life span. The maximum escalation dose in the clinical study is tentatively set at 30 mg/kg, which will also be adjusted if necessary based on the PK, PD and safety data obtained for each dose group of the participants in Australia and China.

### Administration Method and Frequency

21 days (3 weeks) is defined as 1 treatment cycle in this study. All participants will receive an intravenous injection of ALMB-0168 every 3 weeks. Participants will receive multiple cycles of treatment until progressive disease or intolerable toxicity. All participants will be closely monitored for safety.

All participants will be followed in person 28 days after the last treatment, followed by a one-year follow-up to assess long-term outcomes.

### Setting of Dose Escalation Group

Dose escalation levels will be based on ALMB-0168-related adverse events observed during the first cycle of dosing and dose escalation gradients as specified in the dose escalation setup table. If 2 or more participants experience an ALMB-0168-related Grade ≥2 TEAE in the first dosing cycle, or 1 participant experiences an ALMB-0168-related Grade ≥3 TEAE, the magnitude of dose escalation between cohorts may be reduced as necessary, e.g., from 100% dose escalation to no more than 50% dose increase from the previous dose level, as mutually discussed between the investigator and the Sponsor. ALMB-0168 will be escalated in no more than 33% increase at dose levels of 18 mg/kg and above. In case of any problem in the safety assessment, the dose escalation magnitude can be appropriately reduced by the same procedure. It is expected that no more than 7 dose groups will be evaluated during the study. The number and dose level of the actual dose group will depend on the maximum tolerated dose (MTD) and the safety profile observed during the study. If necessary, the investigator and the Sponsor may discuss the intermediate dose escalation levels. See Table 3 below for the dose group setting in the specific dose-escalation stage.

TABLE 3
Specific Dose Escalation Setup of Part I

| | Dose | Percentage Increase from | Number of |
|---|---|---|---|
| Dose Level | (mg/kg) | Previous Dose Level | Participants (n) |
| 1 | 1 | — | 1 ª |
| 2 | 3 | 200 | 1 ª |
| 3 | 6 | 100 | 3-6 |
| 4 | 12 | 100 | 3-6 |
| 5 | 18 | 50 | 3-6 |
| 6 | 24 | 33 | 3-6 |
| 7 | 30 | 25 | 3-6 |
| Dose | XXX mg | | *10 |
| expansion | (MTD) | | |
| at MTD | | | |

^{a} The escalation method of accelerated titration will be used for the first 2 dose groups (1 mg/kg, 3 mg/kg).
One participant will be enrolled in each group, and the dose will be escalated to the next dose group if the participant has no Grade ≥2 TEAE related to the ALMB-0168. If the participant experienced a Grade ≥2 TEAE related to the ALMB-0168, the 3 + 3 dose escalation method will be used for the dose level.
*The MTD cohort will include a maximum of 10 participants, including 3-6 participants in the dose escalation stage, to determine safety.
The dose level and percentage increase from the previous dose level for each dose group will be adjusted based on the study data from the first-in-human clinical study in Australia (ALMB-0168-AU-101).
Dose groups that have completed the DLT observation period can be expanded to a maximum of 8 participants (excluding the MTD expansion cohort) after the investigator and the Sponsor have evaluated the previous clinical data and analyzed the benefits of the participants.

If the safety and tolerability of this dose group are still good when the dose is escalated to the pre-specified maximum dose group, the Investigator and the Sponsor will jointly decide whether to explore the higher dose.

### Dose Escalation Principle

Accelerated titration and standard “3+3” escalation design will be used in the dose escalation stage: The escalation method of accelerated titration will be used for the first 2 dose groups (1 mg/kg, 3 mg/kg), and the dose escalation method of 3+3 design will be used starting from the 3rd dose group (6 mg/kg). At least 3 participants will be enrolled in each dose group, and a judgment will be made within 3 weeks after the 3 participants have completed the first dose at the dose level (DLT observation period) based on adverse events (AEs) related to ALMB-0168 evaluated according to NCI CTCAE Version 5.0. If none of the three participants experience a DLT, the next group will be treated at the next dose level according to the dose escalation rule. At each dose level, if 1 participant experiences a DLT, another 3 participants will be enrolled. Dose escalation will continue until ≥1/3 of the participants experience DLT (when ≥6 participants are assessed) at the dose level. The tolerable dose level before the dose level will be considered the MTD. The selection of RP2D from the tolerated dose range will be based on short- and long-term safety information, as well as findings from preclinical dose-finding studies, PK and anti-tumor activity studies. For dose groups that have completed the DLT observation period, after a comprehensive analysis of the participants' benefits by the investigator and the Sponsor based on the assessment of previous clinical data, they can be expanded to a maximum of 8 participants (excluding the MTD expansion cohort) to further provide information on safety, PK profile and anti-tumor activity, and the safety data of the expanded participants will not be used as the basis for determining the MTD.

### Expansion Stage

In the expansion stage, an expansion study will be conducted in 1-3 dose groups, with a maximum of 60 patients with osteosarcoma enrolled in each dose group to preliminarily explore the efficacy, further describe the safety and PK properties of the ALMB-0168, explore the efficacy, clarify PR2D, etc.

### Definition of End of Study

A participant is considered to have completed the study if he/she has completed all stages of dosing and the end-of-study visit (28 days after the last dose). The end date of the study is defined as the date of the last visit of the last patient in the study. The maximum follow-up time is 12 months. Therefore, the study will be considered closed when the last enrolled patient dies or is followed up for 12 months, whichever is earlier.

### Study Population

### Inclusion Criteria

1. Patients with high-grade osteosarcoma confirmed by histopathology.
2. Defined according to different stages:
a) PART I: Osteosarcoma patients with bone lesions (primary or metastatic) who failed the standard of care, including but not limited to:
   i) Stage III osteosarcoma with bone metastasis, with or without lung metastasis, according to the United States Joint Committee on Cancer (AJCC) bone tumor staging system;
   ii) Unresectable locally advanced osteosarcoma;
   iii) Multifocal osteosarcoma.
b) PART II: patients with high-grade osteosarcoma who failed the standard of care.

The standard of care failure is defined as progressive disease during or within 6 months after chemotherapy with first-line or above chemotherapeutic drugs (including high-dose methotrexate, doxorubicin [cumulative dose ≥350 mg], and cisplatin); for those with progressive disease for more than 6 months, the investigator should conduct a risk-benefit assessment and obtain the consent of the participant or his/her guardian.
3. Patients aged ≥16 years, regardless of gender.
4. Patients with an ECOG (Eastern Cooperative Oncology Group) PS score of 0, 1, or 2.
5. Patients with measurable or non-measurable lesions as per RECIST v1.1. Non-measurable lesions should be confirmed by conventional imaging techniques, including isotope bone scans, CT or MRI. Participants included in PART II should have at least one measurable lesion confirmed by CT or MRI at baseline.
6. Patients with main system functions as follows:
a) Bone marrow reserve: absolute neutrophil count (ANC) ≥1.5×10⁹/L; platelet count ≥75×10⁹/L; hemoglobin ≥9 g/dL, no transfusion in the last 14 days (participants requiring transfusion);
b) Hepatic function: total bilirubin ≤1.5×upper limit of normal (ULN) (unless the patient experiences Grade 1 bilirubin elevation due to Gilbert's disease or a similar syndrome with slow bilirubin binding). Transaminases (AST/SGOT and/or ALT/SGPT)≤3×ULN (liver metastasis <5×ULN);
c) Renal function: Normal serum creatinine ≤1.5 mg/dL (133 μmol/L) or calculated creatinine clearance ≥50 ml/min (Cockroft-Gault formula);
d) Coagulation: defined as an International Normalized Rate (INR)≤2.
7. Female participants of childbearing potential must have a negative serum or urine pregnancy test within 7 days prior to the first dose and must be non-lactating. Male participants with a female partner of childbearing potential and female participants of childbearing potential must use two acceptable methods of contraception, including 1 barrier method, during study participation and within 3 months after the last dose. Male participants must avoid donating sperm during study participation; female participants must avoid donating eggs during the study.
8. Expected survival ≥3 months.
9. Participants (or their guardians) are able to understand the whole process of this study and voluntarily participate in the study and sign the ICF.

### Intervention

### ALAM-0168

The ALMB-0168 is a sterile, pyrogen-free injection of monoclonal antibody against human connexin 43, provided by the Sponsor, AlaMab Therapeutics (Shanghai) Inc. The strength is as follows: each vial contains 6 ml (150 mg) of 25 mg/ml ALMB-0168 monoclonal antibody. The storage condition is: −20±5° C., protected from light and avoiding shaking.

ALMB-0168 will be administered by intravenous injection for at least 30 minutes, and all participants will receive an intravenous drip of ALMB-0168 alone every 3 weeks. Participants will receive multiple cycles of treatment until progressive disease or intolerable toxicity.

Currently, there are no drugs of the same class for ALMB-0168. Chronic active hemorrhagic inflammation and lymphopenia in the central and marginal zones of the spleen were observed in preclinical animal studies. Therefore, participants should be closely observed for infusion reaction, infection, flu-like symptoms, rash, myalgia and arthralgia, fever and chills, and toxicities of the heart, liver, kidney and other organ systems during the study. Participants receiving the first ALMB-0168 infusion must be observed for at least 48 hours post-infusion in an area with resuscitative equipment and emergency medications.

During the study, if participants experience adverse reactions, the investigator should provide necessary supportive treatment according to clinical needs, and allow the suspension of the ALMB-0168 and/or the dose adjustment.

If all ALMB-0168 related AEs occurring in the previous treatment cycle do not return to baseline levels or to Grade 1 on the day of subsequent scheduled treatment, treatment should be delayed and toxic responses graded weekly. The date participants receive the next cycle of ALMB-0168 treatment will be recorded as Day 1 of the next treatment cycle. If all ALMB-0168 related AEs return to baseline levels or to NCI CTCAE Grade 1 within 3 weeks, dosing may be continued.

A participant who requires a treatment delay of more than 21 days due to ALMB-0168 related toxicities occurring in the previous treatment cycle will permanently discontinue study treatment and will complete the study by having an End-of-Treatment Visit 4 weeks after the last treatment.

### Preparation Handling Storage Responsibilities of ALMB-0168

The Sponsor should be responsible for delivering the ALMB-0168 to the study site, and the investigator or investigator's designee should sign to confirm the receipt.

ALMB-0168 can only be used in this study and can only be managed by a special person authorized by the investigator. The investigator/pharmacist in charge of drug administration should distribute, recycle and destroy the ALMB-0168, and maintain accurate records in accordance with the study procedures. The used ALMB-0168 and their packages should be disposed and destroyed as medical wastes in the study site, and the unused or expired drugs should be destroyed by the Sponsor after recovery.

The instructions for rewarming, dilution, mixing and preparation of the ALMB-0168 should be recorded in the corresponding package insert.

A non-randomized, open-label design is adopted in this study.

The usage of ALMB-0168 must be in strict accordance with the clinical protocol regulations, and the dose applied to each participant should be recorded in eCRF.

Investigators in the study site should check and count during drug distribution, recovery and destruction, and sign the documents of drug transfer, handover and destruction for confirmation.

### Dosage Form, Appearance, Package and Label

The label should contain the following:
Protocol No.: ALMB-0168-CN-101
Drug No.:
Drug: ALMB-0168 Injection
Strength: 150 mg, 6 ml/vial
Production Batch No.:
Dosage Form: IV injection
Shelf Life:
Indication: high-grade osteosarcoma after the failure of the standard of care
Instructions for use: See the package insert for details

Storage conditions: −20° C.±5° C., protected from light and shaking before use. The solution for infusion should be used within 28 hours after preparation, including 24 hours when refrigerated at 2° C.-8° C., and 4 hours when administered and temporarily stored at room temperature of 18° C.-26° C. (including the storage and infusion time of the solution for infusion in the infusion bag). If refrigerated, the diluted solution for infusion should be allowed to return to room temperature prior to dosing.

The label should also include the statement that “the drug is provided by AlaMab Therapeutics (Shanghai) Inc. and used for clinical studies only.”

### Product Storage and Stability

The ALMB-0168 should be stored at −20° C.±5° C., protected from light and shaking before use. The solution for infusion should be used within 28 hours after preparation, including up to 4 hours when stored at room temperature of 18° C.-26° C. and up to 24 hours when refrigerated at 2° C.-8° C. (including the storage and infusion time of the solution for infusion in the infusion bag).

The diluted solution for infusion should not be frozen or shaken.

The ALMB-0168 should be ensured to keep stable within the proposed shelf life under the proposed packaging and storage conditions.

### Concomitant Medication and Treatment

Given that ALMB-0168 is a therapeutic monoclonal antibody, there is a low likelihood of drug-drug interactions between ALMB-0168 and small molecule formulations. Since ALMB-0168 is expected to degrade to amino acids and circulate to other proteins, it is unlikely to have an effect on drug metabolizing enzymes or transporters. Medications to treat conditions existing prior to study entry or new conditions emerging during the study may be administered throughout the study. Blood products, painkillers, hypnotics, and other medications for symptomatic management may be administered at the discretion of the investigator. In addition to the drugs listed in the Exclusion Criteria (Section 5.2) and Section 6.5.1, other drugs necessary for the safety and health of participants may be administered at the discretion of the investigator. The investigator may give any necessary concomitant medication or treatment for adequate supportive therapy.

All concomitant medications and treatments during the study should be recorded in the eCRF in detail. For concomitant treatments for osteosarcoma or supportive therapy, the name of the treatment, indication (reason for medication), dose, unit, frequency, start date, and stop date should be collected. For all other concomitant treatments, the treatment name, indication, start date, and stop date should be collected.

Concomitant medications should include not only drugs prescribed by the physicians, but also all OTC, Chinese herbals and vitamins.

### Prohibited/Permitted Food, Drugs and Treatment

During the study, any anti-tumor treatment (including cytotoxic chemotherapy, targeted drugs, radiotherapy, endocrine therapy, and traditional Chinese medicine with anti-tumor indications) and all other clinical investigational drugs are prohibited. If a participant needs such medications, the participant must be withdrawn from the study first.

The following drugs are allowed to be used as appropriate during the study:
(1) Long-term medication required for comorbidities (such as hypertension and diabetes);
(2) Hormone replacement therapy or oral contraceptives not intended for anti-tumor treatment;
(3) On the premise of protecting the interests and safety of the participants, the investigators should give symptomatic and supportive treatment for the toxic response of drugs or to control the tumor symptoms.

### Study Assessment and Procedures

Screening should be completed within 28 days before C1D1. Participants who meet all inclusion criteria and none of the exclusion criteria will be randomized.

Demographic data, including date of birth, gender, age and nationality, will be collected from Day-28 to Day-1.

Body height is measured at screening only. Body weight will be measured during the screening period and on CXD1 (if the body weight examination at screening is completed within 72 h pre-dose, it is not necessary to perform the body weight examination on C1D1.).

### Medical History and Other Past Histories

Medical history refers to clinically significant information (or signs and symptoms, if a diagnosis cannot be confirmed) on previous and concomitant diseases that occurred before signing the ICFs, including past medical history and history of present diseases. Among them, the history of tumor disease includes the time of diagnosis, pathological classification, staging diagnosis and screening stage. All medical histories related to the participant's tumor diagnosis must be traced back to the original diagnosis. In addition, histories of smoking, alcoholism, drug abuse, surgery, or clinically significant abnormalities found in relevant examinations at screening will also be recorded as past history in the original medical records and eCRF.

### Prior Concomitant Medication, Treatment and Prior Anti-Tumor Treatment

Prior medication refers to any medication taken within 28 days prior to the date of investigational drug dosing.

Concomitant medication and treatment refer to the simultaneous administration of two or more drugs/treatments, and are defined in the study as any drug/treatment administered at any time from the simultaneous/contemporaneous first dose of ALMB-0168 to 28 days after the last dose.

At each visit from the initial screening to the end of study, the investigator should inquire about any drug/treatment the participants have taken since the last visit. Any previous medication and concomitant treatment will be recorded in the original medical records and eCRF, and all concomitant medications will be recorded with the following information: drug/non-drug name, cause for medication, dose, unit, dosing frequency, administration route, start date and end date; concomitant non-drug treatment should include the name of non-drug treatment, treatment description, cause for treatment, start date and end date of treatment. If the reasons for use of concomitant medication and treatment comply with the definition of AEs, relevant information should be recorded in the original medical records and eCRF of the participant.

The treatment includes surgery, chemotherapy, radiotherapy, hormonal therapy, or immunotherapy. The following data should be collected: start date and end date of treatment, name of treatment regimen, dose, unit, frequency, best response and reasons for termination of treatment.

### Safety and Other Assessments

The following items are tested in Laboratory.
(1) Hematology: Red blood cell count (RBC), hemoglobin (HGB), hematocrit (HCT), white blood cell count (WBC), platelet count (PLT), counts and percentages of neutrophils, lymphocytes, eosinophils, basophils and monocytes, reticulocyte count, etc.;
(2) Blood chemistry: alanine aminotransferase (ALT), aspartate aminotransferase (AST), alkaline phosphatase (ALP), lactate dehydrogenase (LDH), total bilirubin (TBIL), direct bilirubin (DBIL), total protein (TP), albumin (ALB), urea or blood urea nitrogen (BUN), creatinine (Cr), creatinine clearance (Ccr), glucose (Glu), potassium (K+), sodium (Na+), chloride (Cl−), calcium (Ca2+), magnesium (Mg2+), phosphorus (P), and carbon dioxide;
(3) Myocardial zymogram: creatine kinase (CK) and its isoenzyme (CK-MB);
(4) Urinalysis: pH, specific gravity (SG), glucose (GLU), protein (PRO), red blood cell (RBC), white blood cell (WBC), ketone (KET), occult blood, bilirubin, nitrite, and urobilinogen;
(5) Coagulation function: Prothrombin time (PT), activated partial thromboplastin time (APTT), international normalized ratio (INR), thrombin-time (TT), and plasma fibrinogen (Fbg);
(6) Serum or urine pregnancy test: only for women of childbearing potential;
(7) Virus serology: Hepatitis B test (hepatitis B surface antigen (HBsAg), hepatitis B surface antibody (HBsAb), e antigen (HBeAg), e antibody (HBeAb), and core antibody (HBcAb), further test of HBV-DNA in patients with positive hepatitis B surface antigen), hepatitis C (hepatitis C antibody, further test of HCV-RNA in patients with positive HCV antibody), AIDS (anti-HIV antibody), and syphilis (anti-Treponema pallidum specific antibody).

Hematology (hematology and reticulocyte count), blood chemistry, myocardial zymogram, and urinalysis: Performed at baseline (Day −7 to Day −1), C1D1, C1D8, C1D15, C2D1, C2D8, C2D15, D1 of each subsequent cycle, and at the end-of-study visit. If the CID1 physical examination is expected to be done within 72 hours after the baseline physical examination, it can be omitted.

Coagulation function: Performed at screening, before dosing on D1 of each cycle, and at the end-of-study visit. If the C1D1 physical examination is expected to be done within 72 hours after the baseline physical examination, it can be omitted.

Serum or urine pregnancy test: Performed at screening, before dosing on D1 of each cycle and at the end-of-study visit.

Virus serology: The test will be performed at screening.

The investigator will examine all laboratory findings, assess the participant's changes from baseline in laboratory findings based on the laboratory reference range, determine the clinical significance of each abnormal laboratory value, and record it as “no clinical significance (NCS)” or “clinical significance (CS)” (no marks needed for “normal state”). Abnormal laboratory tests that occur during the study, accompanied by the following symptoms, should be listed as adverse events and recorded in the original medical records and eCRF:
directly withdrawal of participants from the study;
leading to serious adverse consequences;
leading to obvious clinical symptoms;
clinically significant considered by the investigator.

The reviewed laboratory report will be signed and dated by the investigator.

Physical examinations, including skin, mucosa, lymph nodes, head (including skull, ears, eyes, nose, mouth, and throat), neck, chest (including thorax, breasts, lungs, and heart), abdomen (including liver, gallbladder, spleen, kidney, bladder, stomach, and intestines), spine/limbs, nervous system, and others,

The examination will be performed at baseline (Day −7 to Day −1), on C1D1, C1D8, C1D15, C2D1, C2D8, C2D15, on D1 of each subsequent cycle, and at the end-of-study visit. If the C1D1 physical examination is expected to be done within 72 hours after the baseline physical examination, it can be omitted. Compared with the baseline, any clinically significant abnormality should be recorded as an AE.

Vital signs include sitting blood pressure, respiration, pulse and body temperature. Examination of vital signs will be performed at baseline (Day −7 to Day −1), on C1D1, C1D8, C1D15, C2D1, C2D8, C2D15, on D1 of each subsequent cycle, and at the end-of-study visit. If the C1D1 physical examination is expected to be done within 72 hours after the baseline physical examination, it can be omitted. Compared with the baseline, any clinically significant abnormal value should be reported as an AE.

Sitting systolic and diastolic blood pressure should be measured with the same arm each time, and the measurement should be performed after the participant has rested for at least 5 min in a sitting position. All recordings will be made using standard equipment and automatic or manual measurements are acceptable; however, the investigator should continue to perform all assessments of individual participants using the same methods used in the first measurement.

The performance status of the participants will be evaluated according to the ECOG Performance Status Scale (see Appendix I for details). The examination will be performed at baseline (Day −7 to Day −1), on C1D1, C2D1, on D1 of each subsequent cycle, and at the end-of-study visit. If the C1D1 physical examination is expected to be done within 72 hours after the baseline physical examination, it can be omitted.

The examination will be performed at baseline (Day −7 to Day −1), at 30 min (±10 min) pre-dose and 30 min post-dose on C1D1, C1D8, C1D15, at 30 min (±10 min) pre-dose and 30 min (±10 min) post-dose on D1 of each subsequent cycle, and at the end-of-study visit. The investigator may increase the frequency of examinations according to the participant's condition.

Participants should rest for at least 5 min in a supine position before undergoing three 12-lead ECGs.

Performed at baseline (Day −14 to Day −1). In addition, during the study, the investigator may perform examinations according to clinical needs. The participant should rest for at least 5 min in a supine position before UCG.

Examined at baseline (Day −28 to Day −1). During the study, the investigator will perform examinations according to clinical needs.

The schedule of activities is summarized in Table 4 below.

TABLE 4
Schedule of Activities

| | Screening | | | | | | | | | | | End-of- | Follow-up |
|---|---|---|---|---|---|---|---|---|---|---|---|---|---|
| | Period | | During Treatment/Study | | | | | | | | >3 | Study Visit | (12 Months) |
| Procedure/Assessment | Baseline | | Cycle 1 | | | | | Cycle 2 | | | Cycles | 4 Weeks after the | Once every |
| Time Point* | D-28 | D-7 | D1 | D2 | D3 | D8 | D15 | D1 | D8 | D15 | D1 | Last Treatment | 3 months |
| Informed consent ª | X | | | | | | | | | | | | |
| Demographic data | X | | | | | | | | | | | | |
| Inclusion/exclusion | | X | | | | | | | | | | | |
| criteria | | | | | | | | | | | | | |
| Medical history | X | | | | | | | | | | | | |
| Vital signs ^{b} | | X | X | | | X | X | X | X | X | X | X | |
| Physical examination ^{c} | | X | X | | | | X | X | X | X | X | X | |
| Height ^{d} | | X | | | | | | | | | | | |
| Body weight ^{e} | | X | X | | | | | X | | | X | | |
| ECOG score | | X | X | | | | | X | | | X | X | |
| ECHO ^{f} | X | | During the study, the investigator will perform | | | | | | | | | | |
| | | | examinations according to clinical needs. | | | | | | | | | | |
| 12-lead ECG ^{g} | | X | X | | | X | X | X | | | X | X | |
| Assessment of AEs ^{h} | | | X | X | X | X | X | X | X | X | X | X | |
| Assessment of | X | | X | X | X | | X | X | X | X | X | X | X |
| concomitant medication | | | | | | | | | | | | | |
| Complete blood count ⁱ | | X | | | | | X | X | X | X | X | X | |
| Blood chemistry and | | X | | | | | X | X | X | X | X | X | |
| myocardial zymogram ^{j} | | | | | | | | | | | | | |
| Coagulation (PT/aPTT/ | | X | X | | | | | X | | | X | X | |
| INR) ^{k} | | | | | | | | | | | | | |
| Urinalysis ^{l} | | X | X | | | X | X | X | X | X | X | X | |
| Serum or urine | | X | X | | | | | X | | | X | X | |
| pregnancy test ^{m} | | | | | | | | | | | | | |
| Virus serology test ⁿ | | X | | | | | | | | | | | |
| PK blood sampling ^{o} | | | X | X | X | X | X | X | | | X | X | |
| Immunogenicity blood | | | X | | | | | X | | | X | X | |
| sampling ^{p} | | | | | | | | | | | | | |
| Study medication | | | X | | | | | X | | | X | | |
| Evaluation of skeletal | | X | X | | | | | X | | | X | X | X |
| related events (SREs) | | | | | | | | | | | | | |
| Dose and frequency of | | X | X | | | | | X | | | X | X | X |
| morphine use | | | | | | | | | | | | | |
| NRS and EQ-5D scores | | X | X | | | X | X | X | X | X | X | X | X |

| | | | |
|---|---|---|---|
| Bone mineral density | X | BMD will be measured by dual-energy X-ray absorption assay | X |
| (BMD) of the lumbar | | (DXA) every 3 months after treatment. If there are clinical | |
| spine, hip, and | | symptoms, additional examinations can be performed. | |
| femur and neck | | | |
| CT or MRI of brain | | When clinical symptoms of brain metastasis occur or the | |
| | | investigator determines that CT or MRI of the brain is required. | |
| CT of chest, abdomen | X | During the study, the investigator will perform examinations | |
| and pelvis | | according to clinical needs. | |
| CT, enhanced MRI and/ | X | Relevant tumor imaging studies will be performed at Week 6 | X |
| or PET-CTq of lesion | | (end of C2) and every 9 weeks (3 cycles) thereafter. Additional | |
| Isotope bone scan; | X | scans may be performed during the study if clinical symptoms | X |
| technetium Tc 99 | | are found. | |
| Survival and disease | | | |
| response status ^{r} | | | |

Abbreviations:
ADA = Anti-drug antibody;
BP = Blood pressure;
C = Cycle;
CBC = Complete blood count;
CMP = Comprehensive metabolic panel;
D = Day;
diff = Difference;
ECG = Electrocardiogram;
ECHO = Echocardiography;
ECOG = Eastern Cooperative Oncology Group;
PK = Pharmacokinetics;
PS = Performance Status;
RETICS = Reticulocyte count; EOS = End of study
*Description of Procedure/Assessment Arrangements:
Day 1 (D1) of any cycle is defined as the date on which the ALMB-0168 is administered within the given cycle. Procedures/assessments can be arranged within +/− 3 days.
The Day −7 screening/baseline tests and associated assessments can be completed within +/− 7 days (from Day −14 to Day −1).
All pre-dose laboratory tests and other assessments listed for D1 can be completed within 3 days prior to dosing, except that pre-dose ECG on C1D1 should be completed on CIDI at the actual dosing visit.
Visits, laboratory tests and other assessments listed for D2 and D1 of the subsequent cycles can be arranged within +/− 3 days.
For Days 8 and 15 of cycles 1 and 2, visits or/and laboratory or other assessments are allowed to be completed in a +/− 2 days window.
For all imaging procedures, tumor assessments, and bone mineral density tests, according to the SoA, they can be completed in a +/− 5 days window of the specified date.
The end-of-study visit should be a face-to-face visit on the 28th day after the last treatment and can be completed in a +/− 3 days window of the specified date.
^{a} The informed consent must be obtained ≤28 days prior to initiation of study treatment.
^{b} The vital signs, including resting heart rate, respiration, blood pressure and body temperature, will be examined at screening, on C1D1, C1D8, C1D15, C2D1, C2D8 and C2D15, on D1 of each subsequent cycle and at the end-of-study visit. If the C1D1 physical examination is expected to be done within 72 hours after the baseline physical examination, it can be omitted.
^{c} Physical examinations, including skin, mucosa, lymph nodes, head (including skull, ears, eyes, nose, mouth, and throat), neck, chest (including thorax, breasts, lungs, and heart), abdomen (including liver, gallbladder, spleen, kidney, bladder, stomach, and intestines), spine/limbs, and nervous system, will be performed at screening, on C1D1, C1D8, C1D15, C2D1, C2D8, C2D15, on D1 of each subsequent cycle and at the end-of-study visit. If the C1D1 physical examination is expected to be done within 72 hours after the baseline physical examination, it can be omitted.
^{d} Body height: Body height is measured at screening only.
^{e} Body weight: Body weight will be measured during the screening period and on CXD1 (if the body weight examination at screening is completed within 72 h pre-dose, it is not necessary to perform the body weight examination on C1D1.).
^{f} Echocardiography (ECHO): ECHO will be performed within 14 days prior to the initiation of study treatment, and will be performed as needed during the study.
^{g} 12-lead ECG: Three 12-lead ECG examinations will be performed at screening, 30 min (+10 min) pre-dose and 30 min (+10 min) post-dose on C1D1, any time on C1D8 and C1D15, 30 min (+10 min) pre-dose and 30 min (+10 min) post-dose on D1 of each subsequent cycle, the end-of-study visit, and when clinical conditions are found.
^{h} All medical AEs occurring from signing the ICF to 28 days after the last dose will be collected for Assessment. Specific DLT assessments will be performed from dosing until the end of C1 (C1D21) or prior to the scheduled dosing on C2D1 (i.e., C1D22).
ⁱ Complete blood count includes hematology and reticulocyte count. Hematology: red blood cell count (RBC), hemoglobin (HGB), hematocrit (HCT), white blood cell count (WBC), platelet count (PLT) and counts and percentages of neutrophils, lymphocytes, eosinophils, basophils and monocytes; It will be performed at screening, on CID1, CID8, CID15, C2D1, C2D8, C2D15, on DI of each subsequent cycle and at the end-of-study visit. If
the C1D1 physical examination is expected to be done within 72 hours after the baseline physical examination, it can be omitted. Samples for hematology will be collected prior to each dose.
^{j} Blood chemistry: alanine aminotransferase (ALT), aspartate aminotransferase (AST), alkaline phosphatase (ALP), lactate dehydrogenase (LDH), total bilirubin (TBIL), direct bilirubin (DBIL), total protein (TP), albumin (ALB), urea or blood urea nitrogen (BUN), creatinine (Cr), creatinine clearance (Ccr), glucose (Glu), potassium (K+), sodium (Na+), chloride (Cl−), calcium (Ca2+), magnesium (Mg2+), phosphorus (P), and carbon dioxide. Myocardial zymogram includes creatine kinase (CK) and its isoenzyme (CK-MB). It will be performed at screening, on C1D1, C1D8, C1D15, C2D1, C2D8, C2D15, D1 of each subsequent cycle, and at the end-of-study visit. If the C1D1 physical examination is expected to be done within 72 hours after the baseline physical examination, it can be omitted.
^{k} Coagulation markers (PT/aPTT/INR): prothrombin time (PT), activated partial thromboplastin time (APTT), international normalized ratio (INR), thrombin time (TT), and plasma fibrinogen (Fbg), which will be tested at screening, before dosing on D1 of each cycle, and at the end-of-study visit. If the time of C1D1 test is within 72 h after the baseline test, it is not necessary to perform the C1D1 test. Thrombin activator time/prothrombin time test at screening will be performed in a local laboratory using standard methods.
^{l} Urinalysis: pH, specific gravity (SG), glucose (GLU), protein (PRO), red blood cell (RBC), white blood cell (WBC), ketone (KET), occult blood, bilirubin, nitrite, and urobilinogen. It will be performed at screening, on C1D1, C1D8, C1D15, C2D1, C2D8, and C2D15, on D1 of each subsequent cycle and at the end-of-study visit. If the C1D1 physical examination is expected to be done within 72 hours after the baseline physical examination, it can be omitted.
^{m} Serum or urine pregnancy test: The serum pregnancy test will be performed at the baseline test (to be completed within 7 days of the first dose, and it is not necessary to perform the C1D1 test if the time of the baseline test is within 72 h), and the urine pregnancy test can be performed at other time points. For women of childbearing potential, the pregnancy test will be performed before dosing on D1 of each cycle and at the end-of-study visit.
ⁿ Virus serology: Test at baseline. Hepatitis B test (hepatitis B surface antigen (HBsAg), hepatitis B surface antibody (HBsAb), e antigen (HBeAg), e antibody (HBeAb), and core antibody (HBcAb), further test of HBV-DNA in patients with positive hepatitis B surface antigen), hepatitis C (hepatitis C antibody, further test of HCV-RNA in patients with positive HCV antibody), AIDS (anti-HIV antibody), and syphilis (anti-treponema pallidum specific antibody);
^{o} PK blood sample collection time: 1. All participants in the escalation stage and the first 10 participants in the expansion stage will be intensively sampled. Intensive sampling time points: Pre-dose (within 30 min) and immediately (within 5 min), 2 h (+15 min), 4 h (+30 min) and 8 h (+1 h) post-dose on C1D1; 24 h (+2 h) post-dose on C1D2 and 48 h (+2 h) pose-dose on C1D3; 168 h (+6 h) post-dose on C1D8 and 336 h (+12 h) post-dose on C1D15; pre-dose (within 30 min) and immediately post-dose (within 5 min) on D1 of each subsequent cycle; at the end of the study. 2. The rest of the participants in the expansion stage will be sparsely sampled. Sparse sampling time points: Pre-dose (within 30 min) and immediately post-dose (within 5 min) on D1 of each cycle; at the end of the study visit. 3. The number and time points of PK sampling may change with the actual condition of the study. When infusion reactions or serious adverse events occur, the investigator may cooperate with the laboratory of the same visit to test the collected blood and perform an unscheduled PK sampling.
^{p} Blood sample collection time for immunogenicity: Pre-dose (within 30 min) on D1 of cycles 1, 2, 3, 5, and 7 and at the end of the study (4 weeks after the last treatment). Samples will be used for anti-drug antibody (ADA) test.
^{q} Both RECIST and ICDS criteria will be used for response evaluation in the study. It should be emphasized to the radiologist that in addition to the size assessment, ICDS criteria will also be used for density assessment. If the investigator deems it necessary, imaging procedures other than those specified (such as PET-CT) can be performed and the response can be evaluated according to RECIST criteria.
^{r} All participants will receive clinic visits every 3 months until 12 months after the last dose to follow up on disease status, subsequent anti-tumor treatment, and survival status. These visits will have a time window of +7 days.

### Response Evaluation

### Tumor Assessment

Imaging procedures in appropriate tumor-associated anatomical areas including: CT, MRI contrast-enhanced scan and/or PET-CT of tumor-related sites, CT or MRI of brain, and technetium-99 isotope bone scan.

The results of participants within 4 weeks prior to dosing are acceptable in the tumor assessments performed during the screening period. During the screening period, the investigator should select the appropriate tumor assessment method according to the participant's condition, and the tumor assessment during the follow-up period should be conducted in the same method as for the baseline assessment. During the study, the first tumor assessment will be performed at Week 6 (end of C2) and every 9 weeks (3 cycles) thereafter. Additional scans may be performed during the study if clinical symptoms are found.

The response evaluation will be performed using both RECIST v1.1 (Appendix III) and ICDS criteria (Appendix VI). It should be emphasized to the radiologist that in addition to the size assessment, ICDS criteria will also be used for density assessment.

### Study Endpoints

Overall response rate (ORR): defined as the proportion of patients with the best response of complete response (CR) or partial response (PR) (i.e., CR+PR) from the start of the ALMB-0168 dosing to study withdrawal, as evaluated according to the Response Evaluation Criteria in Solid Tumors (RECISTv1.1, as detailed in Appendix III; ICDS, Appendix VI). Participants who are first evaluated as the CR or PR should be confirmed after at least 4 weeks.

Progression-Free Survival (PFS): refers to the time from the start of the ALMB-0168 to the first recorded date of progressive disease (PD) or death, whichever occurs first.

6-month progression-free survival rate (6m-PFSR) is defined as the percentage of participants who survive without PD within 6 months from the start of the ALMB-0168.

Duration of response (DoR): defined as the time from the first evaluation as CR or PR (best response) until the first day when the participant presents evidence of tumor progression or dies.

Time to response (TTR): defined as the time from the first dose to the first occurrence of objective response (PR or CR).

Disease control rate (DCR): defined as the percentage of participants with the response of PR+CR and with stable disease (SD) after treatment among the evaluable participants.

Overall survival (OS): defined as the time from the start of study treatment to death from any cause.

Rate of change from baseline in alkaline phosphatase (ALP) or lactate dehydrogenase (LDH).

Skeletal related events (SREs): including changes from baseline in the number of pathological fractures, spinal cord compression, malignancy-associated hypercalcemia, disease-modifying radiotherapies or surgeries for bone lesions, etc.

Change from baseline in the NRS score (see Appendix IV for details).

Change from baseline in the EQ-5D score (see Appendix V for details).

Bone mineral density of lumbar spine, hip, and femur and neck: Measured by dual-energy X-ray absorption assay (DXA), with changes from baseline collected.

Change from baseline in dose and frequency of morphine use.

The time points for evaluation of efficacy indicators including ALP, LDH, SRE, NRS, EQ-5D, bone mineral density and morphine use are detailed in the SoA table.

### Response Criteria

The investigator will perform imaging assessments according to the Response Evaluation Criteria in Solid Tumors (RECIST v1.1) and ICDS criteria during the screening period and each treatment cycle. The same imaging technique as the baseline should be used throughout the study and the lesions can be divided into measurable lesions and non-measurable lesions. If the participant's tumor-related symptoms are aggravated during treatment, tumor assessment can be performed in advance as judged by the investigator. For a participant who discontinues treatment prior to progressive disease, a tumor assessment will be performed every 3 months after the last dose.

Participants showing progressive disease by imaging examination should be withdrawn from the study.

In rare cases, if the investigator believes that the treatment benefits the participant, the participant with radiographically confirmed PD may also continue the study treatment. It is required to consult the sponsor first.

If deemed necessary by the investigator, imaging procedures other than those specified (e.g., PET-CT) may be performed and response evaluation may be performed according to the PERCIST criteria, and the above data may be included and summarized for efficacy assessment.

Skeletal related events (SREs), dose and frequency of morphine use, NRS, and EQ-5D scores should be collected at the respective visits (see SOA for details).

### Pharmacokinetic, Immunogenicity and Biomarker Assessment

### PK Indicators

Blood samples for PK will be collected from the contralateral arm at the following time points as specified in the SoA. The number and time points of PK sampling may change with the actual condition of the study. When infusion reactions or serious adverse events occur, the investigator may cooperate with the laboratory of the same visit to test the collected blood and perform an unscheduled PK sampling.

All participants in the escalation stage and the first 10 participants in the expansion stage will be intensively sampled. Intensive sampling time points: Pre-dose (within 30 min) and immediately (within 5 min), 2 h (±15 min), 4 h (±30 min) and 8 h (±1 h) post-dose on C1D1; 24 h (±2 h) post-dose on C1D2 and 48 h (±2 h) pose-dose on C1D3; 168 h (±6 h) post-dose on C1D8 and 336 h (±12 h) post-dose on C1D15. Pre-dose (within 30 min) and immediately post-dose (within 5 min) on D1 of each subsequent cycle; at the end of the study visit. 2. The rest of the participants in the expansion stage will be sparsely sampled. Sparse sampling time points: Pre-dose (within 30 min) and immediately post-dose (within 5 min) on D1 of each cycle; at the end of the study visit.

PK indicators: Cₘₐₓ, tₘₐₓ, AUC₀₋ₜ, C_{trough}, and CL, V_{z}, λ_{z}, t_{1/2}, AUC_{0-inf} (for participants subject to intensive sampling only).

### Immunogenicity Indicators

To evaluate the immunogenicity of ALMB-0168, blood samples should be tested for anti-drug antibodies. Samples from participants to analyze immunogenicity/drug concentrations may also be used to assess safety or efficacy, to address issues that arise during or after the study, or to assist the Sponsor or Sponsor's designee in evaluating relevant biomarkers.

Blood sample collection time points: Pre-dose (within 30 min) on D1 of Cycles 1, 2, 3, 5, 7 and at the end of the study (4 weeks after the last treatment). Samples will be used for anti-drug antibody (ADA) test.

Blood samples for the determination of anti-ALMB-0168 antibodies (ADA) will be collected from the contralateral arm at the time points specified in the SoA.

### Statistical Considerations

There are no formal statistical hypotheses for this study and the statistical analysis results are exploratory.

### Sample Size Estimation

Phase I part of the study consists of accelerated titration and standard “3+3” dose escalation. One to 6 participants are enrolled in the first 2 dose groups, and 3 to 6 participants are enrolled in the third and subsequent dose groups, with a maximum of 7 dose groups, including participants administered at the MTD level. Dose groups that have completed the DLT observation period can be expanded to a maximum of 8 participants (excluding the MTD expansion cohort) after the investigator and the Sponsor have evaluated the previous clinical data and analyzed the benefits of the participants. Therefore, the maximum number of participants should not exceed 58.

A maximum of 180 participants will be enrolled in the Phase II dose expansion, subject to the final number of participants.

### Analysis Population

Full Analysis Set (FAS): includes all participants who have received at least one dose of the ALMB-0168 after successful enrollment according to the basic principle of intention-to-treat (ITT). FAS is used in demographic and baseline characteristics analysis.

Per Protocol Set (PPS): Defined as a subset of the participants in the Full Analysis Set (FAS). Participants who meet the inclusion and exclusion criteria, have at least one post-baseline tumor assessment, have medication compliance between 80% and 120%, and have no serious violations of the clinical study protocol.

Pharmacokinetic Analysis Set (PKS): Participants who receive at least one dose of the ALMB-0168 and have at least 1 measurable plasma concentration. Reasons for excluding a participant or a participant's plasma concentration from the PKS include but are not limited to the following factors affecting the plasma concentration (e.g., wrong treatment, concomitant medication), improper processing of blood samples, and incorrect blood sample testing.

Safety Analysis Set (SS): The SS will include all participants who receive at least one dose of the ALMB-0168 and receive the safety evaluation. Unless otherwise specified, all analyses will be based on the SS. The SS set is defined as consistent with the FAS set.

Anti-Drug Antibody Set (ADA Set, ADAS): Participants who receive at least 1 dose of the ALMB-0168 with available ADA data.

Efficacy Evaluable Analysis Set (EES): includes all participants who have received at least one dose of the study drug and have received at least one post-dose efficacy evaluation.

Pharmacokinetic Concentration Set (PKCS): It includes all participants who have received at least one dose of the study drug and have at least one evaluable plasma concentration data.

Pharmacokinetic Parameter Set (PKPS): It includes all participants who have received at least one dose of the study drug and have at least one evaluable PK parameter.

Immunogenicity Analysis Set (IS): It includes all participants who have received at least one dose of the study drug and have evaluable immunogenicity data at baseline and at least one evaluable immunogenicity data post-baseline.

Biomarker Analysis Set: It includes all enrolled participants who have received at least one dose of the study drug and have at least one biomarker data.

### Statistical Analysis

The detailed summary and statistical analysis methods for the data collected from this study will be included in the Statistical Analysis Plan (SAP). The SAP will be developed after the protocol and CRF are finalized and will be finalized before the database lock. The SAP will specify and describe in detail all planned statistical analyses based on the main characteristics of the protocol. Any changes to the protocol that, in the judgment of the sponsor or the principal investigator, have a significant impact on the statistical analysis plan will require a new revision of the SAP to align with the study protocol.

For statistical description, the statistical description of categorical variables will be expressed by the number of participants and percentage (%); the statistical description of continuous variables will be expressed by the number of participants, missing value, mean, standard deviation, median, quartile, minimum value and maximum value.

For participant disposition, enrollment, medication, dropout or exclusion, and dataset division of participants of each group are summarized and presented with the participant disposition flow chart.

Statistically describe the demographic data and baseline characteristics: For continuous variables in baseline data, the number of participants, mean, standard deviation, median, minimum and maximum will be calculated; for count and ranked data, the number of participants and percentages will be calculated. The participant's demographic data, baseline characteristics, past medical history and treatment history are detailed in a table.

### Pharmacokinetics and Immunogenicity

### 1. Pharmacokinetic Analysis

Mean (Mean±SD) dose-time curves of ALMB-0168 will be plotted by dose group. Individual PK parameters of participants will be listed and descriptive statistical analysis (mean, median, range, standard deviation, coefficient of variation, etc.) will be performed for PK parameters.

PK parameters: Cₘₐₓ, tₘₐₓ, C_{trough}, and CL, V_{z}, λ_{z}, t_{1/2}, AUC₀₋ₜ, AUC_{0-inf} (for participants with intensive sampling only)

### 2. Immunogenicity Analysis:

The number and percentage of ADA-positive participants will be summarized and presented.

### Safety Analysis

### 1. AEs and Drug-Related AEs.

For the analysis of AEs and drug-related AEs, the number of participants, the number of AEs and percentage of AEs, drug-related AEs, AEs leading to termination of the study, AEs leading to death and SAEs in the participants of each group will be summarized.

System organ class (SOC) and preferred term (PT) of AEs and drug-related AEs will be coded according to the MedDRA dictionary, and the number of participants, the number of AEs, and percentage of AEs and drug-related AEs will be summarized by SOC/PT:

The number of participants, the number of AEs, and percentage of AEs and drug-related AEs will be classified by SOC/PT and severity. A class of AE of one participant will be counted only once under the highest severity in the same term (SOC or PT).

All AEs (including AEs during the non-medication period), drug-related AEs, SAEs, AEs leading to termination of the study, AEs leading to death, and adverse events of special interest (AESIs) will be listed separately.

### 2. Laboratory Tests

point after baseline and last visit time points. The measured value at each time point, the minimum and maximum of the post-baseline measured results, the observed value of the last visit and the change value from baseline should be statistically analyzed according to dose groups and the total.

The normal and abnormal changes of each indicator before and after treatment will be compared through the clinical judgment cross-tabulation before and after treatment. Laboratory test indicators will be listed by group in detail.

### 3. Vital Signs

The statistical time points for vital signs examination include baseline, each visit time point after baseline and visit time points of early withdrawal. The descriptive statistical results of baseline, visit time points after baseline and at the end of the study will be statistically analyzed by group and the total, and then the normal and abnormal changes of each indicator before and after treatment will be compared through the clinical judgment cross-tabulation before and after treatment (if applicable).

### 4. Physical Examination and ECG

Physical examination items: general condition, skin, head and neck, chest, abdomen, back, limbs, nerves, and spirit. Compare the normal and abnormal changes of each dose group before and after treatment through the clinical judgment cross-tabulation before and after treatment.

ECG examination includes heart rate, PR, QRS and QTc. Statistically analyze the baseline, each visit time point after baseline, minimum and maximum of post-baseline measured results, observed value of the last visit and the change value from baseline by the dose group and the total. Compare the normal and abnormal changes of each dose group before and after treatment through the clinical judgment cross-tabulation before and after treatment.

List the detailed listings of physical examinations and ECG examinations by group.

### 5. ECOG Score

Statistical time points for the ECOG score include baseline, each post-baseline visit, and the last visit. Descriptive statistics will be performed at baseline, post-baseline visit time points and at the end of treatment by cohort, and ECOG score changes before and after treatment will be compared by cross-tabulation before and after treatment.

### Efficacy Analysis

The response can be evaluated as PD, SD, CR, and PR according to RECIST V1.1 and ICDS criteria. Specific analysis indicators include but are not limited to the following:
Overall response rate (ORR): The number and percentage of participants with objective tumor response (CR or PR) will be summarized.
Overall survival (OS): refers to the time from randomization to death from any cause. Or to the last follow-up visit for patients lost to follow-up; or to the end of follow-up for participants who are still alive at the end of the study. The last date of contact is the latest date on which the participant is known to be alive.
Progressive disease-free survival (PFS): defined as the period from the first dose to progressive disease or death, whichever occurs earlier. For participants with no progressive disease or death, the date of the last tumor assessment will be taken as the endpoint and their data will be treated as censored values.
6-month progression-free survival rate (6m-PFSR): The percentage of participants who survive without PD within 6 months from the start of the ALMB-0168.
Time to response (TTR): defined as the time from the first dose to the first occurrence of objective tumor response (CR or PR).
Duration of response (DoR): the time from the first evaluation as CR or PR (best response) until the first day when the participant presents evidence of tumor progression or dies.
Disease control rate (DCR): defined as the percentage of participants with the response of PR+CR and with stable disease (SD) after treatment among the evaluable participants.
The response in the participants will be evaluated according to RECIST 1.1 and ICDS criteria, ORR and DCR will be calculated, and 95% confidence interval (CI) will be calculated for ORR and DCR by Clopper-Pearson exact method; DoR, PFS and TTR will be estimated by Kaplan-Meier method, and the median DoR, PFS, TTR, censoring rate, quartiles and their 95% CIs will be listed respectively and Kaplan-Meier curves will be plotted.
Analysis of Numeric Rating Scale (NRS) score: Describe the NRS score and change from baseline at each visit.
Europe Health-related Quality of Life Questionnaire 5 Dimensions (EQ-5D) score: Describe the EQ-5D score and change from baseline at each visit.
If deemed necessary by the investigator, imaging procedures other than those specified in the protocol (e.g., PET-CT) may be performed and response evaluation may be performed according to the PERCIST criteria, and these data may be included and summarized for efficacy assessment.

### Appendices

### Appendix I ECOG Performance Status Scale

The ECOG Performance Status Scale is shown as in Table 5.

TABLE 5
ECOG Performance Status Scale

| Grade | Performance Status |
|---|---|
| 0 | Fully active, able to carry on all pre-disease performance without |
| | restriction. |
| 1 | Restricted in physically strenuous activity, but ambulatory and |
| | able to carry out work of a light or sedentary nature, such as light |
| | house work or office work. |
| 2 | Ambulatory and capable of all self-care, but unable to carry out |
| | any work activities; up and about more than half of waking hours. |
| 3 | Capable of only limited self-care; confined to bed/chair more |
| | than half of waking hours. |
| 4 | Completely disabled; cannot carry out any self-care; totally |
| | confined to bed/chair. |
| 5 | Death |

### Appendix II Cardiac Function Classification of New York Heart Association (NYHA)

The Cardiac Function Classification of New York Heart Association is shown in Table 6.

TABLE 6
NYHA Cardiac Function Classification

| Grading | Symptoms |
|---|---|
| Grade I | The patient has cardiac organic disease, but routine activity is |
| | not restricted, and general activity does not cause fatigue, |
| | palpitation, dyspnea, or angina. |
| Grade II | The physical activity of the patient is slightly restricted, and |
| | there is no subjective symptom during rest, but fatigue, |
| | palpitation, dyspnea or angina may occur during general |
| | activities. |
| Grade III | The physical activity of the patient is obviously limited, and |
| | general activities lighter than usual cause fatigue, palpitation, |
| | dyspnea or angina. |
| Grade IV | The patient cannot engage in any physical activity. At rest, |
| | there are also symptoms of cardiac insufficiency, which are |
| | aggravated after physical activity. |

### Appendix III Excerpt of Response Evaluation Criteria in Solid Tumors (RECIST 1.1)

### History of RECIST Criteria

Evaluating the change of tumor burden is an important feature of clinical evaluation of malignant tumor treatment. Both tumor reduction (objective response) and time to progression are important endpoints for determination in a clinical study of malignant tumors. To screen for new antineoplastic agents, years of research evidence has backed tumor reduction as an endpoint in Phase II study. These studies suggest that for a variety of solid tumors, drugs that contribute to tumor reduction in some patients are likely (though imperfect) to be demonstrated to improve the overall survival of the patient or to have other opportunities for entry into event evaluation in a randomized Phase III study. At present, the objective response is more reliable than any other biomarker in evaluating efficacy in Phase II screening study. Furthermore, in phase II and phase III clinical studies in advanced patients, the time to progression (or progression-free survival) is gradually being used as the endpoint for determining efficacy, which is also based on anatomic measurements of tumor size.

However, the two endpoints for determination of tumor, objective response and time to progression, are only of value if they are supported by criteria that they are widely accepted and easily used based on the anatomy of tumor burden. Tumor response criteria were first published by the World Health Organization (WHO) in 1981 and are mainly used in trials where tumor response is the primary endpoint. WHO standards introduce the concept of performing an overall evaluation of tumor burden by calculating the sum of the two-dimensional size of the lesion to evaluate the response of treatment by the change from baseline during treatment. However, for more than a decade after the publication of the criteria, the oncology groups and pharmaceutical companies using the criteria often modified them to adapt to new technologies or to propose unclear points of the original literature, which led to confusion in the interpretation of study results. In fact, the application of various response criteria resulted in widely varying treatment outcomes for the same treatment method. In response to these issues, an international working group was established in the mid-19th century to standardize and simplify the response criteria. The new criteria, also known as RECIST (Response Evaluation Criteria in Solid Tumors), was published in 2000. The key features of the initial version of RECIST included the definition of the smallest measurable lesion size, a description of the number of follow-up lesions (up to 10; up to 5 per organ), and an overall evaluation of tumor burden using one-dimensional (instead of two-dimensional) measurements. These criteria were widely adopted by academic groups, oncology groups and pharmaceutical industry, and the initial endpoint of the criteria was objective response or progressive disease. In addition, NMPA accepted RECIST as appropriate criteria for these evaluations.

### Purpose of the Guideline

The guideline describes a standard measurement method for a solid tumor in clinical studies of malignant tumors in adults and children and defines objective criteria for determining changes in tumor size. It is expected that these criteria will be used in all studies with objective response as the main study endpoint, and studies taking stable disease, tumor progression, or analytical progress times as indicators, because all measures of efficacy are based on the evaluation of the anatomical tumor burden and its changes in the study. The proportion of patients meeting the criteria for endpoints that predict the efficacy of a drug or treatment regimen is not assumed in this paper: Those definitions depend on the type of malignant tumors in the ongoing study and the particular drug under study. The study protocol must include an appropriate statistical section that defines the number of samples for the study and the efficacy parameters on which the inclusion criteria are based. This guideline not only provides the definition and criteria for determining tumor response, but also puts forward suggestions for the criteria report of clinical study results which take tumor response as the endpoint of the study.

Although these guidelines can be used in the study of malignant brain tumors, the criteria for evaluating response in this field have also been published separately. Since international guidelines for the evaluation of the response of lymphoma have also been published separately, this guideline is not used in the study of malignant lymphoma.

Finally, many oncologists rely on repeated imaging studies to follow up on patients' tumor lesions in their daily clinical practice and decide on further treatment regimens based on objective indicators and symptoms. These RECIST guidelines should generally not be used in clinical decision-making, unless the oncologist believes that they are suitable for treatment decision-making.

### Baseline Tumor Measurement

At baseline, tumor lesions/lymph nodes are classified as measurable and non-measurable, as below.

### 1. Measurable Lesions

Tumorous lesion: tumor lesion. The tumor lesion has at least one diameter that can be accurately measured (recorded as the longest diameter of the measurement plane), and its minimum length is as follows:
10 mm for computed tomography (CT) (CT slice thickness not greater than 5 mm).
10 mm measured by calipers for clinical examination (lesions that cannot be accurately measured by calipers should be recorded as non-measurable).
20 mm for chest X-ray.

Malignant lymph nodes: When evaluated by CT (CT slice thickness is recommended to be not greater than 5 mm), the minor axis of the lymph node must reach 15 mm to be considered pathologically enlarged and measurable. Only minor axis lengths are measured and followed up for pre-operation and follow-up.

### 2. Non-Measurable Lesions

All other lesions, including small lesions (with a maximum diameter of less than 10 mm or the minor axis of pathological lymph node of 10 mm to less than 15 mm) and truly non-measurable lesions. Lesions considered to be truly measurable include pia mater lesions, ascites, pleural or pericardial effusion, inflammatory breast disease, lymph of skin/lung infiltration, peritoneal dissemination, as well as abdominal mass/abdominal organ enlargement that cannot be confirmed by repeated imaging techniques and physical examination.

### 3. Special Considerations Regarding Measurable Lesions

Bone lesions, cystic lesions, and lesions that have been locally treated should be specifically noted, as outlined below: Bone lesions:
Bone scans, positron emission tomography (PET) scans, or plain radiographs are not considered appropriate imaging techniques for measuring bone lesions. However, these techniques can be used to confirm the existence or disappearance of bone lesions.
For osteolytic lesions or mixed osteolytic/osteogenic lesions with a defined soft tissue component, they can be evaluated as measurable lesions with tomographic imaging techniques such as CT or MRI, provided the soft tissue component meets the above definition of measurability.
Osteogenic lesions are non-measurable. Cystic lesions:
Lesions that meet the criteria for the definition of simple cysts on radiologic imaging should not be considered malignant (neither measurable nor non-measurable) because they are defined as simple cysts.
If cystic lesions represent cystic metastatic lesions and meet the measurability defined above, they can be considered measurable. However, if non-cystic lesions are present in the same patient, non-cystic lesions should be preferred as target lesions.

Lesions that have been locally treated:
A tumor lesion located in a previously irradiated area or at a site subjected to other local regional treatments is generally considered a non-measurable lesion, unless it shows unequivocal progression. The study protocol should detail the conditions under which such lesions would be considered measurable.

### Measurement Method Specification

### 1. Measurement of Lesions

All measurements should be recorded in metric notation, using calipers if clinically evaluated. All baseline evaluations must be performed as close to the start of treatment as possible, and less than four weeks earlier.

### 2. Measurement Methods

The same determination methods and techniques should be used to describe each identified and reported lesion at the baseline and follow-up phases. Normally, imaging examinations (instead of clinical examinations) should be carried out, unless the lesion is found to be unsuitable for imaging at follow-up.

Clinical lesion: Only superficial lesions (such as subcutaneous nodes) with a caliper measurement of 10 mm or more in diameter are considered measurable. For cases of skin lesions, it is recommended to use color photographs with the proportion of lesion size measured. As mentioned earlier, when the lesion can be examined both clinically and by imaging, imaging should be used, because the imaging evaluation is more objective and can be used for the final review of clinical studies

Chest X-ray examination: Compared with chest X-ray examination, chest CT is preferred, especially when progressive disease is used as an important endpoint, because CT scans are more sensitive than X-ray examinations in identifying new lesions. However, if the X-ray reveals a clear lesion boundary, which is surrounded by an inflated lung, it is considered measurable.

CT, MRI: CT is currently the most effective and repeatable test method to evaluate the response of lesions. As defined by the guideline, CT slice thickness should be not more than 5 mm for measurable lesions. When the CT slice thickness exceeds 5 mm, the minimum measurable lesion should be twice the slice thickness. MRI can also be used under certain circumstances (such as whole body scan).

Ultrasonography: Ultrasonography is not suitable for evaluating lesion size and should not be applied as a measurement method. Ultrasonography cannot be completely reproduced between two adjacent observations. Moreover, the results depend on the examiner. Technique and measuring results cannot be secured the same for one test and its next. If new lesions are detected by ultrasonography during the course of the study, CT or MRI validation is recommended. If CT radiation exposure is a concern, MRI can be used instead to detect the lesion to be examined.

Endoscopy, laparoscopy: These techniques are not recommended for objective tumor evaluation. However, they are beneficial in confirming complete pathological response with biopsy or in determining complete response or recurrence after surgical resection.

Tumor markers: tumor markers cannot be used alone to evaluate the objective response of the tumor. However, when the tumor markers begin to rise above the upper limit of normal, the markers must be reduced to the normal range if used to determine the complete response of the patient. Because tumor markers are disease-specific, indications for testing for a particular disease should be included in the protocol. Specific guidelines on CA-125 changes (ovarian cancer recurrence) and PSA changes (prostate cancer recurrence) have been published. In addition, the Gynecologic Cancer Intergroup developed the CA-125 progression criteria for clinical studies with objective tumor evaluation indicators for first-line treatment of ovarian cancer.

Cytology, histology: These techniques can often be used to distinguish between partial and complete responses in individual cases (e.g., residual benign tumor lesions in the tumor type of reproductive cell tumor) if required by the clinical study protocol. When exudation is known to be a potential adverse consequence of therapy (e.g., certain paclitaxel-based chemotherapeutic agents or angiogenesis inhibitors), measurable tumor lesions that meet the criteria for response or stable disease require cytologic confirmation of oncologic exudation that occurs or worsens during therapy, in order to distinguish between response (or stable disease) and progressive disease.

### Response Evaluation of Tumor

### 1. Evaluation of All Tumors and Measurable Lesions

In order to evaluate objective response or possible future progression, it is necessary to perform a baseline evaluation of the total burden of tumors in all tumor lesions, as a reference for subsequent measurements. In the clinical protocol with objective response as the main treatment endpoint, only patients with measurable lesions at baseline will be enrolled. Measurable lesions are defined as the presence of at least one measurable lesion. For studies with progressive disease (time to progression or degree of progression on a fixed date) as the main treatment endpoint, it must be clear from the protocol inclusion criteria whether it is limited to patients with measurable lesions or whether patients without measurable lesions can also be enrolled.

### 2. Baseline Records of Target and Non-target Lesions

When more than one measurable lesions are present at baseline evaluation, all lesions should be recorded and measured, totaling no more than 5 (no more than 2 per organ), as target lesion representing all involved organs (i.e., patients with only one or two cumulative organs should take a maximum of two or four target lesions as baseline measurement lesions).

Target lesions must be selected based on lesion size (maximum diameter), representative of all involved organs, and measurements must be repeatable. In the case that the largest lesion cannot be measured repeatedly, another largest lesion which can be measured repeatedly.

Lymph nodes can still be imaged in normal anatomy without tumor metastasis and therefore require special attention. Pathological lymph nodes defined as measurable nodules or even target lesions must meet the following criteria: short diameter ≥15 mm measured by CT. Only short diameter needs to be detected at baseline. Radiologists usually use the short diameter of a nodule to determine whether there is tumor metastasis. Nodule size is generally expressed by two-dimensional data of imaging examinations (axial plane is commonly used for CT scanning, and axial plane, sagittal plane or coronal plane is selected for MRI). The short diameter is determined by the minimum value taken. For example, a 20 mm×30 mm abdominal nodule with a short-axis diameter of 20 mm can be considered a malignant, measurable nodule. In this example, 20 mm is the measurement of the nodule. Nodules ≥10 mm but <15 mm in diameter should not be considered target lesions. Nodules <10 mm are not considered pathological and should not be recorded or further observed.

The calculated sum of the diameters of all target lesions (including the maximum diameter of non-nodular lesion and the short-axis diameter of nodular lesion) will be the sum of the baseline diameters. If the diameter of lymph nodes is involved, as noted above, only the short diameter will be included. The sum of baseline diameters will be used as a reference for baseline disease.

All remaining lesions including pathological lymph nodes should be considered non-target lesions without measurement but should be recorded at baseline evaluation as follows: “Present,” “Absent” or, in a few cases, “Unequivocal Progression.” Extensive non-target lesions in the same organ may be recorded on the case report form as a single item, such as numerous enlarged pelvic lymph nodes or extensive liver metastases.

### 3. Response Evaluation Criteria

### 3.1. Response Evaluation on Target Lesions

Complete response (CR): All target lesions are absent, and the short-axis diameter of any pathological lymph node (whether it is a target lesion or not) must be <10 mm.

Partial response (PR): The sum of the diameters of all target lesions is reduced by at least 30% relative to the sum of baseline diameters.

Progressive disease (PD): The sum of the diameters of all target lesions is increased by at least 20% relative to the sum of the diameters of the minimum lesion during the study (including the sum of baseline diameters of lesion if it is the minimum). In addition to the relative increase of 20%, the sum of the diameters must also demonstrate an absolute increase of at least 5 mm (note: the presence of one or more new lesions can also be considered progressive).

Stable disease (SD): Based on the sum of the diameters of the minimum lesion during the study, the lesion reduction is not a PR, and the lesion increase is not a PD.

### 3.2. Considerations for Response Evaluation on Target Lesions

When target lesions are present in lymph nodes:

The measured short-axis diameter should be recorded (at the same anatomic surface as the baseline measurement), even if the lymph nodes are resolved to below 10 mm in the study.

This means that when target lesions are present in lymph nodes, the sum of the diameters of target lesions will not be 0 even if complete response criteria are met, since a lymph node with a short-axis diameter <10 mm is considered normal. Case report forms or other methods of data collection may be designed to have nodular target lesions recorded separately. For complete response, each node must have a short-axis diameter <10 mm. For PR, SD, and PD, the sum of the diameters of target lesions will include the actual short-axis diameters of nodes.

When target lesions are too small to be measured:

For all lesions (nodular and non-nodular) recorded at baseline in the study, their actual measurements must be recorded in subsequent evaluations, even if the lesion is very small (e.g., 2 mm). However, some lesions or lymph nodes recorded as target lesions at baseline have too weak signals on CT scans to be exactly measured by the radiologist, and they can be reported as too small to be measured. In this case, it is important to record one measurement in the case report form. If it is the opinion of the radiologist that the lesion may disappear, the measurement can be recorded as 0 mm. If the lesion does exist with a weak signal, a default value of 5 mm should be assigned (Note: This rule is not applicable to normal lymph nodes since they usually have a definable size and are frequently surrounded by fat such as in the retroperitoneum; however, if the lymph node does exist with a too weak signal to be measured, a default value of 5 mm should also be assigned). The default value of 5 mm is based on the thickness of the CT tomography scan (the default value of 5 mm will not change with this thickness). The measurement of such lesions (too small to be measured) may lack repeatability and should be assigned a default value to prevent evaluation as a false response or false progression due to measurement error. Again, if the radiologist is able to provide an actual measurement, even less than 5 mm, it should be recorded.

When lesions split or coalesce on treatment

When non-nodular lesions are separated, the maximum diameters of all fragments must be summed for the sum of diameters of target lesions. Similarly, when lesions coalesce, the long-axis diameters can be retained, which helps to obtain the maximum diameter of each individual lesion prior to coalescence. If the lesions have completely coalesced such that they are no longer separable, the vector of the maximum diameter in this instance should be the maximum diameter of the coalesced lesion.

### 3.3. Evaluation of Non-Target Lesions

This section defines the criteria for response to non-target lesions tumors. While some non-target lesions are actually measurable, they need not be measured and only need to be qualitatively evaluated at the time points specified in the protocol.

Complete Response (CR): All non-target lesions are resolved and tumor markers return to normal. All lymph nodes are non-pathological in size (short diameter <10 mm).

Non-complete response/non-progressive disease (non-CR or non-PD): There are one or more non-target lesions and/or the tumor marker is constantly above the normal level.

Progressive Disease (PD): Existing non-target lesions show unequivocal progression.

Note: The presence of one or more new lesions is also considered a progressive disease.

### 3.4. Considerations for Evaluation of Progression of Non-Target Lesions

The definition of progression of non-target lesions is additionally explained below:

When patients have measurable lesions: In this case, for an unequivocal progression of non-target lesions, the non-target lesions are worsened to such a degree that the treatment must be discontinued. Even if the target lesions are evaluated as stable disease or partial response, the overall tumor burden has increased sufficiently to support discontinuation of treatment. Mild increases in one or more non-target lesions are usually not sufficient to be considered as unequivocal progression. In the case of stable disease or partial response of target lesions, it is extremely rare for overall tumor progression to be defined solely by changes in non-target lesions.

When none of the non-target lesions in the patient is measurable: This occurs when the inclusion criteria do not specify that a measurable lesion must be present in some phase III studies. The overall evaluation is still based on the above criteria, but there are no measurable data for lesions in this case. Worsening of non-target lesions is not easy to evaluate (by definition: All lesions are truly non-measurable). Therefore, when changes in non-target lesions lead to an increase in the overall disease burden equivalent to the occurrence of progressive disease in the target lesions, an unequivocal progression should be defined based on non-target lesions, and an effective test should be established for evaluation. For example, an increase in tumor burden represents an additional 73% increase in volume (which is equivalent to a 20% increase in the diameter of measurable lesions). Another example is peritoneal exudation from “minimal” to “large”; lymphatic lesions ranged from “local” to “widespread”; or “change in the treatment method based on sufficient evidence” described in the protocol. In case of an unequivocal progression, the patient should be considered generally to have had progressive disease at that time point. Although it is ideal to have non-measurable lesions evaluated by objective criteria, there may be lesions that are not objectively evaluable in nature and therefore must be significantly enlarged.

### 3.5. New Lesions

The presence of new malignant lesions denotes progressive disease; therefore, some comments on new lesions are important. There are no specific criteria for the identification of new lesions by imaging; however, the finding of a new lesion should be unequivocal. For example, progression cannot be attributed to differences in imaging techniques, changes in imaging morphology, or other lesions other than tumors (e.g., some so-called new bone lesions are just the cured original lesions or the recurrent original lesions). It is important when the patient's baseline lesions show partial or complete response. For example, necrosis of a liver lesion may be documented on a CT report as a new cystic lesion, while it is actually not.

Lesions that are detected at follow-up and are not identified at baseline will be considered new, suggesting progressive disease. For example, in a patient with a visceral lesion identified at baseline, when metastasis is found on CT or MRI of the head, the intracranial metastasis will be considered a basis for progressive disease, even if he does not undergo a head examination at baseline.

If a new lesion is equivocal for its small size, continued therapy and follow-up evaluation are required to confirm whether it is a new lesion. If repeated examinations confirm that it is definitely a new lesion, the time to progression should be calculated from the time it is first discovered.

FDG-PET assessment generally requires additional examination for confirmation, so it is reasonable to combine FDG-PET and CT findings to evaluate the progression (especially for new suspected diseases). New lesions can be identified by FDG-PET examination according to the following procedures: A negative FDG-PET result at baseline and a positive FDG-PET result at follow-up suggest progressive disease.

No FDG-PET at baseline and a positive FDG-PET result at follow-up:

If the new lesion identified by a positive FDG-PET at follow-up is confirmed by CT, there will be a progressive disease.

If the new lesion identified by a positive FDG-PET at follow-up is not confirmed by CT, additional CT will be needed for confirmation (if confirmed, the time to progression should be calculated from the time when the abnormality is found in early FDG-PET).

If the positive FDG-PET at follow-up confirms an existing lesion identified by CT and the lesion is not progressing on imaging, there will be no progressive disease.

### 4. Evaluation of Best Overall Response

The evaluation of best overall response is a record of the best response from the start of the study to the end of the study, taking into account any necessary conditions for confirmation. On occasion, the response will not be confirmed until the end of treatment, so the protocol should specify whether the response evaluation at the end of treatment is included in the evaluation of best overall response. The protocol must specify how any new treatment that precedes progression affects the best response. The best response of patients depends primarily on the results of target and non-target lesions and the presence or absence of new lesions, as well as the nature of the study, protocol requirements, and outcome measures. Specifically, in non-randomized studies where the response is the primary endpoint, confirmation of PR or CR is a must to determine which is the best overall response.

4.1. It is assumed that response evaluation will be conducted at each specific time point of the protocol. Table 1 provides a summary of the overall response at each time point for patients with measurable disease at baseline.

If the patient has no measurable lesion (no target lesions), the evaluation can be found in Table 2.

### 4.2. Description on Missing Evaluation and Non-Evaluable Cases

If no lesion imaging/measurement is done at a specific time point, the patient will be non-evaluable at that time point. If only partial lesions can be evaluated at one time, usually the case will be non-evaluable at that time point, unless a convincing argument can be made that the missing lesions will not affect the response evaluation at the specified time point. This is likely to occur in the setting of progressive disease. For example, if a patient had 3 lesions with a sum of 50 mm in diameter at baseline and only 2 lesions are evaluable later with a sum of 80 mm, the patient will be evaluated as to have progressive disease, regardless of the effect of missing lesions.

### 4.3. Best Overall Response: All Time Points

The best overall response can be determined if all information about the patient is available.

Evaluation of the best overall response when the confirmation of complete or partial response is not required in the study: The best response in the study is the best response at all time points. For example, if a patient has SD in the first cycle, PR in the second cycle, and PD in the last cycle, the best overall response will be PR. When the best overall response is SD, it must also meet the protocol-specified minimum time from baseline. If the minimum time criterion is not met, the best overall response of SD will not be accepted, and it will be determined by the subsequent evaluation. For example, if a patient has SD in the first cycle and PD in the second cycle but the minimum time for SD is not met, the best overall response will be PD. Similarly, the patient lost to follow-up after being evaluated as SD in the first cycle will be considered as non-evaluable.

Evaluation of the best overall response when the confirmation of complete or partial response is required in the study: A complete or partial response can be confirmed only if each participant meets the criteria for a partial or complete response specified in the study and if the response is confirmed at a subsequent time point as specified in the protocol (generally 4 weeks later). In this case, the description of the best overall response is provided in Table 3.

### 4.4. Special Notes on Response Evaluation

When nodular lesions are included in the overall evaluation of target lesions, and the nodules decrease to “normal” size (<10 mm), there will still be scan reports on lesion size. To avoid over-evaluation, measurements will be recorded even if the nodules are normal as reflected by an increase in nodule size. As previously mentioned, this means that 0 will not be recorded on the CRF for the participants with complete response.

If response confirmation is required during the study, repeated “non-measurable” time points will complicate the best response evaluation. The analysis plan for the study must address how responses will be judged by these missing data. For example, in most studies, the response of PR-NE-PR of a participant can be considered confirmed.

Symptomatic progression should be reported when the participant has a global deterioration requiring discontinuation of treatment without objective evidence. Objective progression should be evaluated as far as possible even after discontinuation of treatment. Symptomatic deterioration is not a descriptor of objective response, but the reason for discontinuation of treatment. The objective response of such participants will be evaluated by the description of target and non-target lesions as shown in Tables 1 to 3.

Cases defined as early progression, early death and non-evaluable are exceptions of the study and should be clearly described in each protocol (depending on treatment interval and treatment cycle).

In some cases, it may be difficult to distinguish a local lesion from normal tissue. When

the evaluation of a complete response is based on such a definition, we recommend that the biopsy be performed prior to the response evaluation of the local lesion complete response.

When the abnormal imaging results of local lesions in some participants are considered to represent the fibrosis or scar formation of the lesion, FDG-PET is used as the evaluation standard similar to biopsy to confirm the complete response. The use of FDG-PET, in this case, should be prospectively described in the protocol, supported by reports from the specialized medical literature on this case. However, it must be acknowledged that both FDG-PET and biopsy may lead to false positive results at the evaluation of complete response due to their own limitations (including resolution/sensitivity).

TABLE 7
Time-Point Response: Participants with Target Lesions
(With or without Non-target Lesions)

| Target | Non-target | New | Overall |
|---|---|---|---|
| Lesions | Lesions | Lesions | Response |
| CR | CR | None | CR |
| CR | Non-CR/Non-PD | None | PR |
| CR | Non-evaluable | None | PR |
| PR | Non-progressive or | None | PR |
| | non-fully evaluable | | |
| SD | Non-progressive or | None | SD |
| | non-fully evaluable | | |
| Non-fully | Non-progressive | None | NE |
| evaluable | | | |
| PD | Any cases | Yes or No | PD |
| Any cases | PD | Yes or No | PD |
| Any cases | Any cases | Yes | PD |
| CR = complete | PR = partial | SD = stable | PD = progressive |
| response | response | disease | disease |
| | | | NE = Non- |
| | | | evaluable |

In SD cases, at least one post-enrollment follow-up measurement must meet SD criteria and the follow-up and enrollment should be separated by at least 6-8 weeks.

TABLE 8
Time-point Response - Participants with Non-target Lesions Only

| Non-target Lesions | New Lesions | Overall Response |
|---|---|---|
| CR | None | CR |
| Non-CR or non-PD | None | Non-CR or non-PD |
| Non-fully evaluable | None | Non-evaluable |
| Equivocal PD | Yes or No | PD |
| Any cases | Yes | PD |

Note:
For non-target lesions, “non-CR/non-PD” refers to response superior to SD. Since SD is
increasingly used as the endpoint for response evaluation, non-CR/non-PD response is established for cases where no lesion is measurable.

For equivocal findings of progression (e.g., very small and uncertain new lesions; cystic changes or necrosis in existing lesions), treatment may continue until the next evaluation. If in the next evaluation, progressive disease is confirmed, the date of progression should be the date of previously suspected progression.

TABLE 9
Best Overall Response to be Confirmed for CR and PR

| Overall | Overall | |
|---|---|---|
| Response at | Response at | |
| the First | Subsequent | |
| Time Point | Time Points | Best Overall Response |
| CR | CR | CR |
| CR | PR | SD, PD or PRª |
| CR | SD | If SD persists for enough time, it is SD, |
| | | otherwise, it should be PD |
| CR | PD | If SD persists for enough time, it is SD, |
| | | otherwise, it should be PD |
| CR | NE | If SD persists for enough time, it is SD, |
| | | otherwise, it should be NE |
| PR | CR | PR |
| PR | PR | PR |
| PR | SD | SD |
| PR | PD | If SD persists for enough time, it is SD, |
| | | otherwise, it should be PD |
| PR | NE | If SD persists for enough time, it is SD, |
| | | otherwise, it should be NE |
| NE | NE | NE |

Note:
CR = complete response, PR = partial response, SD = stable disease, PD = progressive disease, NE = non-evaluable. Superscript ″a″: If CR actually occurs at the first time point and any disease occurs at subsequent time points, then even if the participant meets the PR criteria relative to baseline, the response will still be PD at subsequent time points (because the disease will reappear after CR). The best response depends on whether SD occurs at the shortest treatment interval. However, sometimes the response is evaluated as CR at the first time point, but scans at subsequent time points suggest a small lesion, so in fact, the response of participants should be PR rather than CR at the first time point. In this case, the CR evaluated at the first time point should be changed to PR and the best response should be PR.

### 5. Frequency of Tumor Reevaluation

The frequency of tumor reevaluation during treatment depends on the treatment regimen and should be consistent with the type and schedule of treatment. However, in Phase II studies where the benefit of treatment is unclear, follow-up every 6 to 8 weeks (timed to coincide with the end of a cycle) is reasonable, and the length of the interval may be adjusted in special regimens or cases. The protocol should specify which tissue sites are to be evaluated at baseline (usually those most likely to be involved with metastasis of the tumor type under study) and how often evaluations are repeated. Normally, target and non-target lesions are evaluated at each time. In some optional cases, certain non-target lesions may be evaluated less frequently. For example, bone scans may need to be repeated only when CR is identified in target diseases or when progression in bone lesions is suspected.

At the end of treatment, reevaluation of tumors depends on whether the response rate or the time to an event (progression/death) is used as the endpoint of the clinical study. For the time to an event (e.g., TTP/DFS/PFS), the routine repeated evaluation specified in the protocol is required. In randomized comparative studies, in particular, the scheduled evaluations should be outlined in the schedule (e.g., 6 to 8 weeks on treatment or 3 to 4 months after treatment) and should not be affected by delays in treatment, dosing intervals, and any other events that may cause an imbalance in treatment arms in the timing of disease evaluation.

### 6. Response Evaluation/Confirmation of Response Period

### 6.1. Confirmation

In non-randomized clinical studies where the response is the primary endpoint, confirmation of PR and CR is a must to ensure that the response is not the result of an evaluation error. This also allows for a sound interpretation of results in the context of historical data, but the response described in the historical data of these studies should also be confirmed. However, in all other cases, such as randomized studies (phase II or III) or in studies where stable disease or progressive disease is the primary endpoint, response confirmation is no longer required because it is of no value in the interpretation of the study results. Eliminating the need for response confirmation, however, would make a central review that prevents offset even more important, especially in non-blind studies.

In the case of SD, at least one measurement should meet the SD criteria specified in the protocol within the shortest time interval after the study starts (generally not less than 6-8 weeks).

6.2. The overall response period is calculated from the time when CR or PR criteria are met for the first time (whichever is first measured) to the time when the recurrent or progressive disease is objectively documented (the minimum measurement recorded in the study is used as a reference for progressive disease). The overall complete response period is calculated from the time when CR criteria are met for the first time to the time when the recurrent or progressive disease is objectively documented.

### 6.3. Stable Disease Period

It is calculated from the beginning of treatment (in randomized studies, from the time of randomization) to progressive disease, by reference to the sum of the minimum measurements in the study (if the baseline sum is minimum, it is used as a reference for PD calculation). The clinical relevance of the stable disease period varies with studies and diseases. If the proportion of patients achieving stable disease for a minimum period of time is a study endpoint in a particular study, the protocol should specify the minimum time interval between two measurements as defined in the SD.

Note: The response period, stable disease period, and PFS are influenced by the frequency of follow-up after baseline evaluation. It is not within the scope of this guideline to define a standard frequency of follow-up. The frequency of follow-up should be determined based on disease typing and staging, treatment cycle, standard practice, etc. The limitations in the accuracy of the measurement endpoints should be taken into account if comparisons between studies are needed.

### 7. Progression-Free Survival (PFS)/Percentage Progression-Free (PPF)

This guideline focuses on the use of objective response as a study endpoint in Phase II clinical study. In some cases, the response rate may not be optimal for evaluating the potential anticancer activity of a new drug/regimen. In these cases, PFS/PPF at demarcated time points may be considered a suitable surrogate to provide an initial signal of the biological activity of a new drug. However, it is clear that in an uncontrolled study, these evaluations will be questioned, as seemingly valuable observations may be related to biological factors such as the screening of patients rather than the effect of the drug intervention. Therefore, Phase II clinical studies with these study endpoints are best designed with randomized control. However, since the clinical manifestations of some tumors are consistent (generally, always poor) all the time, the non-randomized studies are reasonable. However, in these cases, due to the lack of positive control, the evidence of efficacy should be recorded with caution when expected PFS or PPF is evaluated.

Note: age is in years and weight is in kilograms (kg).

### Appendix IV Numeric Rating Scale (NRS) Score

Scores 0-10 represent different degrees of pain. Ask the patient about the degree of pain, and make a mark, or ask the patient to circle the figure that best represents the degree of pain.
0: No pain.
1-3: Mild pain (not affect sleep).
4-6: Moderate pain.
7-9: Severe pain (unable to fall asleep or wake up with pain in sleep).
10: Smarting.

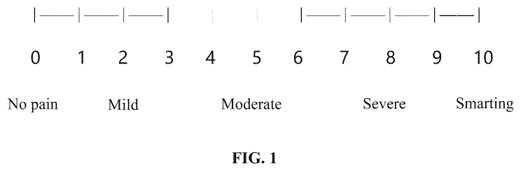

Different degrees of pain is also shown in FIG. 1.

### Appendix V Europe Health-Related Quality of Life Questionnaire 5 Dimensions (EQ-SD)

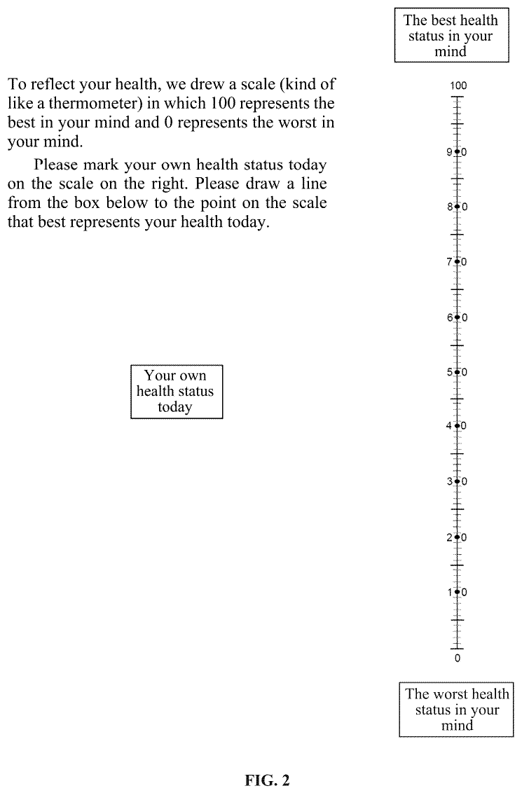

The EQ-5D form is shown below. Patient name, randomized No., date of visit and the corresponding dosing ofthe visit are documented. The related scale is shown in FIG. 2.

TABLE 10
EQ-5D Questionnaire
Please select one of the following options (E1-E5) that best reflects
your health today:

| | | |
|---|---|---|
| E1 | Action | |
| | (1) I have no problems walking | □ |
| | (2) I have slight problems walking | □ |
| | (3) I am unable to walk | □ |
| E2 | Self-care | |
| | (1) I have no problems taking care of myself | □ |
| | (2) I have slight problems washing, brushing, bathing or | □ |
| | dressing myself | |
| | (3) I am unable to wash, brush, bath or dress myself. | □ |
| E3 | Usual activities (such as work, study, housework, family | |
| | or leisure activities) | |
| | (1) I have no problems doing my usual activities | □ |
| | (2) I have slight problems doing my usual activities | □ |
| | (3) I am unable to do my usual activities | □ |
| E4 | Pain/Discomfort | |
| | (1) I have no pain or discomfort | □ |
| | (2) I have moderate pain or discomfort | □ |
| | (3) I have extreme pain or discomfort | □ |
| E5 | Anxiety (nervous, worried, upset, etc.)/Depression | |
| | (lack of interest, loss of fun, loss of spirit, etc.) | |
| | (1) I am not anxious or depressed | □ |
| | (2) I am moderately anxious or depressed | □ |
| | (3) I am extremely anxious or depressed | □ |
| E6 | To reflect your health, we have drawn a thermometer- | Results: |
| | like scale in which 100 represents the best health you | |
| | can imagine and 0 represents the worst health you can | |
| | imagine.See ″VAS Scale″ on the back | |

### Appendix VI ICDS (Modified Inverse Choi Density Size Criteria) Evaluation Criteria

TABLE 11
ICDS Evaluation Criteria

| Complete | |
|---|---|
| Response | |
| (CR) | All lesions disappeared |
| Partial | ≥10% reduction in size (% Δ Choi SLD) or >5% reduction |
| Response | in CT density from baseline, no new lesion, and no |
| (PR) | significant progression of non-measurable lesions |
| Stable | Not meeting the criteria for CR, PR, or PD; no symptom |
| Disease | worsening due to tumor progression. |
| (SD) | |
| Progressive | ≥10% increase in tumor size (Choi SLD) and CT density |
| Disease | not meeting the criteria for PR; any new lesion on |
| (PD) | CT/MRI; new intratumoral nodules or enlargement of |
| | existing intratumoral nodules. |
| Unevaluable | Unable to receive CT/MRI, or evaluation. If the density |
| (UE): | and size measurements show that the target lesion is not |
| | evaluable and does not meet the criteria for PD, the |
| | evaluation result is not evaluable. |

It should be evaluated by contrast-enhanced spiral CT or MRI.
CT attenuation of each tumor should be measured in Hounsfield units (HUs) during the portal venous phase.
SLD stands for sum of the longest diameters.

### Example 3. A Multicenter, Single-Arm, Open-Label Phase I/II Clinical Study to Evaluate the Safety and Efficacy of ALMB-0168 in Patients with Osteosarcoma—Results

24 participants were enrolled to receive humanized monoclonal antibody (ALMB-0168) involved at doses of 1 mg/kg, 3 mg/kg, 6 mg/kg, 12 mg/kg, 18 mg/kg, 24 mg/kg, 30 mg/kg or 36 mg/kg once every 3 weeks. Baseline characterization is shown in Table 12.

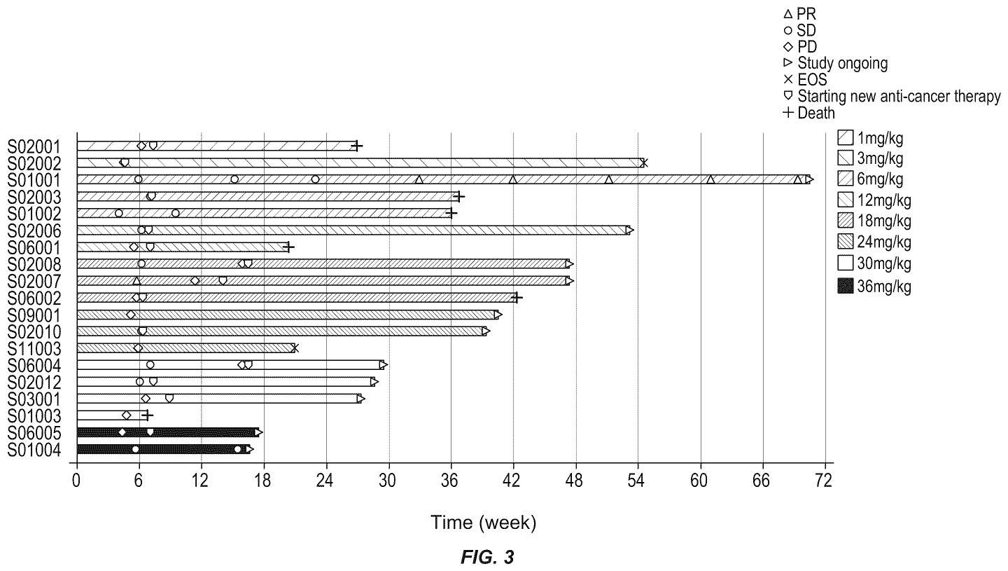

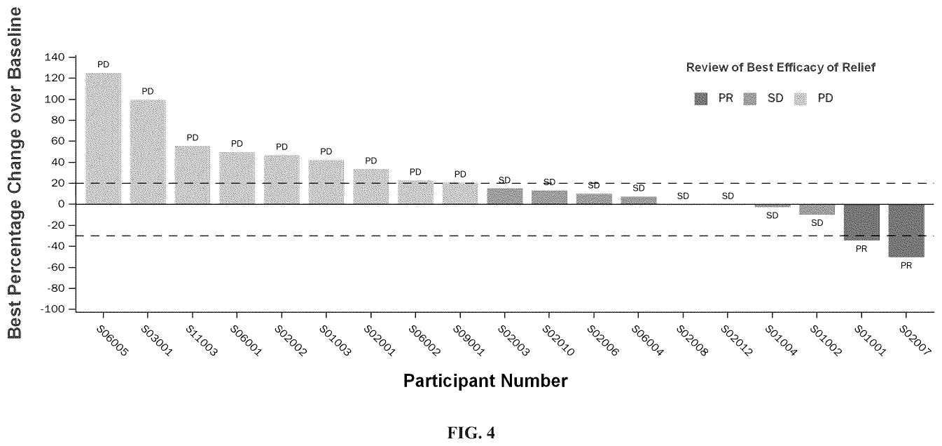

Of the 24 participants, 3 of were not evaluated and withdrawn from the study, 2 experienced symptomatic progression, and 19 were evaluable (2 PR, 8 SD, and 9 PD). The disease progression of the 19 participants is shown in FIG. 3. The best change of investigated-determined target lesion diameter of the 19 participants is shown in FIG. 4.

The overall response rate was 10.5% (2/19) and the DCR was 52.6% ((2+8)/19) for EES. Efficacy data of dose groups: 1 mg/kg (1 PD), 3 mg/kg (1 PD), 6 mg/kg (1 PR, 2 SD), 12 mg/kg (1 SD, 1 PD, 1 NE), 18 mg/kg (1 PR, 1 SD, 1 PD), 24 mg/kg (1 SD, 2 PD), 30 mg/kg (3 PD, 1 SD, 1 NE)), 36 mg/kg (2 PD, 1 SD, 1 NE) are shown in Table 13.

None of the 24 participants experienced DLT events. A total of 80 drug-related adverse events were observed, of which: adverse events with an incidence of ≥10% were: anemia (9 participants, 11.25%) and proteinuria (8 participants, 10.00%), only one Grade ≥3 treatment related adverse event, and no drug-related serious adverse event. The treatment emergent adverse events are summarized in Table 14.

According to the pharmacokinetic data, the exposure of ALMB-0168 in participants after a single intravenous drip increased with dose proportionally within the dose range of 1-36 mg/kg, with a mean peak concentration (Cₘₐₓ) of about 19.7-691 μg/mL and a mean area under the drug-time curve (AUC₀₋ₜ) of about 4365-123795 h*μg/ml. ALMB-0168 was eliminated slowly in vivo, with a mean elimination half-life of about 8.54-14.8 days at a mean elimination rate of about 8.75-13.0 ml/h. It presents a limited tissue distribution, with a mean apparent volume of distribution of about 3.55-4.88 L.

TABLE 12
Baseline Characterization of 24 Participants

| Randomized | | Pathological | Dose | | | Best |
|---|---|---|---|---|---|---|
| No. | Sex | Diagnosis | (mg/kg) | ECOG | Target Lesion | Therapy |
| S02001 | F | telangiectatic | 1 | 0 | pancreas body + pancreas | PD |
| | | osteosarcoma | | | tail | |
| S02002 | M | classic osteosarcoma | 3 | 0 | left iliac soft tissue + right | PD |
| | | | | | lung | |
| S02003 | M | small cell | 6 | 0 | pancreas tail + left lung | SD |
| | | osteosarcoma | | | | |
| S01001 | F | classic osteosarcoma | 6 | 1 | midshaft of right femur | PR |
| S01002 | M | classic osteosarcoma | 6 | 1 | anteromedial tissue of left | SD |
| | | | | | ilium | |
| S02006 | F | classic osteosarcoma/ | 12 | 0 | left acetabular soft tissue + | SD |
| | | osteoblastic | | | right lung | |
| | | osteosarcoma | | | | |
| S06001 | M | classic osteosarcoma/ | 12 | 1 | right lower lung + Carina | PD |
| | | chondroblastic | | | lymph node + Left iliac | |
| | | osteosarcoma | | | subcutaneous mass | |
| S12001 | M | classic osteosarcoma/ | 12 | 2 | right lower limb + left lobe | NE |
| | | osteoblastic | | | of liver | |
| | | osteosarcoma | | | | |
| S02007 | M | classic osteosarcoma | 18 | 0 | Left lung | PR |
| S02008 | M | classic osteosarcoma | 18 | 0 | non-target only | SD |
| S06002 | M | classic osteosarcoma/ | 18 | 1 | left 7th rib + left 2nd rib | PD |
| | | fibroblastic | | | | |
| | | osteosarcoma | | | | |
| S11003 | M | classic osteosarcoma/ | 24 | 1 | soft tissue of right scapula | PD |
| | | chondroblastic | | | | |
| | | osteosarcoma | | | | |
| S09001 | M | classic osteosarcoma | 24 | 1 | left chest wall mass+ left | PD |
| | | | | | upper lung | |
| S02010 | F | classic osteosarcoma/ | 24 | 0 | Left lung | SD |
| | | osteoblastic | | | | |
| | | osteosarcoma | | | | |
| S02011 | M | classic osteosarcoma | 30 | 1 | left distal femur | PD |
| S14002 | M | classic osteosarcoma | 30 | 0 | non-target only | NE |
| S01003 | M | osteosarcoma | 30 | 2 | right lung upper lobe + right | PD |
| | | | | | pleura + left clavicle lymph | |
| | | | | | node + right diaphragm + | |
| | | | | | left ischium | |
| S06004 | F | classic osteosarcoma | 30 | 0 | right lung upper lobe + right | SD |
| | | | | | lung lower lobe + right | |
| | | | | | mandibular bone | |
| S02012 | M | classic osteosarcoma | 30 | 0 | non-target only | SD |
| S03001 | F | classic osteosarcoma | 30 | 1 | thoracic 6th vertebra + right | PD |
| | | | | | upper lung + left humerus | |
| S06005 | M | classic osteosarcoma | 36 | 0 | right iliac soft tissue mass + | PD |
| | | | | | right ischial soft tissue mass | |
| S11005 | M | classic osteosarcoma | 36 | 1 | left ischium + left lung lower | NE |
| | | | | | lobe + right lung lower lobe | |
| S15001 | F | osteosarcoma | 36 | 1 | right hip + ilium + pelvic | PD |
| | | | | | cavity +lung + skull | |
| S01004 | F | small cell | 36 | 2 | Lumbar 4 vertebrae and | SD |
| | | osteosarcoma | | | surrounding areas | |

TABLE 13
Efficacy Analysis of 24 Participants (8 Dose Groups)

| | 1 mg | 3 mg | 6 mg | 12 mg | 18 mg | 24 mg | 30 mg | 36 mg | n = 24 | n = 19 |
|---|---|---|---|---|---|---|---|---|---|---|
| | (n = 1) | (n = 1) | (n = 3) | (n = 3) | (n = 3) | (n = 3) | (n = 6) | (n = 4) | (FAS) | (EES) |
| CR | | | | | | | | | 0 | 0 |
| PR | | | 1 | | 1 | | | | 2 | 2 |
| SD | | | 2 | 1 | 1 | 1 | 2 | 1 | 8 | 8 |
| PD | 1 | 1 | | 1 | 1 | 2 | 2 | 1 | 9 | 9 |
| Symptomatic | | | | | | | 1 | 1 | 2 | |
| PD | | | | | | | | | | |
| NE | | | | 1 | | | 1 | 1 | 3 | |
| ORR | 0.0% | 0.0% | 33.3% | 0.0% | 33.3% | 0.0% | 0.0% | 0.0% | 8.3% | 10.5% |
| DCR | 0.0% | 0.0% | 100.0% | 33.3% | 66.6% | 33.3% | 33.3% | 25.0% | 41.7% | 52.6% |

TABLE 14
Summary of TEAE of the Safety Analysis Set

| | 1 mg/kg | 3 mg/kg | 6 mg/kg | 12 mg/kg | 18 mg/kg | 24 mg/kg | 30 mg/kg | Total |
|---|---|---|---|---|---|---|---|---|
| | (N = 1) | (N = 1) | (N = 3) | (N = 3) | (N = 3) | (N = 3) | (N = 6) | (N = 20) |
| | n (%) | n (%) | n (%) | n (%) | n (%) | n (%) | n (%) | n (%) |
| ≥1 TEAE | 1 (100) | 1 (100) | 3 (100) | 3 (100) | 3 (100) | 3 (100) | 5 (83.3) | 19 (95.0) |
| SAE | 0 | 0 | 0 | 0 | 0 | 1 (33.3) | 0 | 1 (5.0) |
| TEAE causing permanent | 0 | 0 | 0 | 0 | 0 | 0 | 0 | 0 |
| medicine withdrawal | | | | | | | | |
| TEAE causing reduced | 0 | 0 | 0 | 0 | 0 | 0 | 1 (16.7) | 1 (5.0) |
| dose | | | | | | | | |
| Death-causing TEAE | 0 | 0 | 0 | 0 | 0 | 0 | 0 | 0 |
| CTCAE ≥ grade 3 TEAE | 0 | 0 | 0 | 0 | 0 | 1 (33.3) | 1 (16.7) | 2 (10.0) |
| TEAE causing exit of trial | 0 | 0 | 0 | 0 | 0 | 0 | 0 | 0 |
| Research drug-related | 0 | 0 | 2 (66.7) | 2 (66.7) | 3 (100) | 3 (100) | 4 (66.7) | 14 (70.0) |
| TEAE | | | | | | | | |
| SAE | 0 | 0 | 0 | 0 | 0 | 0 | 0 | 0 |
| TEAE causing permanent | 0 | 0 | 0 | 0 | 0 | 0 | 0 | 0 |
| medicine withdrawal | | | | | | | | |
| TEAE causing reduced | 0 | 0 | 0 | 0 | 0 | 0 | 0 | 0 |
| dose | | | | | | | | |
| Death-causing TEAE | 0 | 0 | 0 | 0 | 0 | 0 | 0 | 0 |
| CTCAE ≥ grade 3 TEAE | 0 | 0 | 0 | 0 | 0 | 1 (33.3) | 0 | 1 (5.0) |
| TEAE causing exit of trial | 0 | 0 | 0 | 0 | 0 | 0 | 0 | 0 |

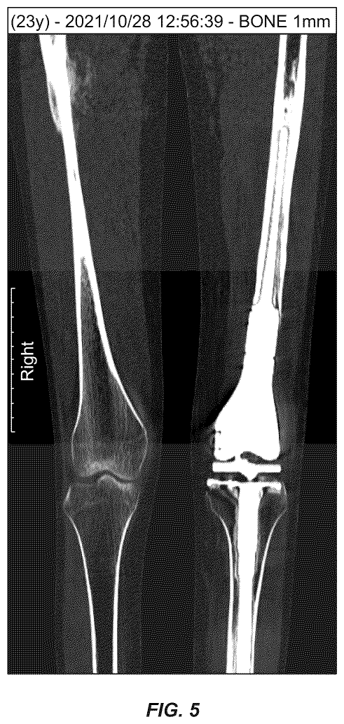

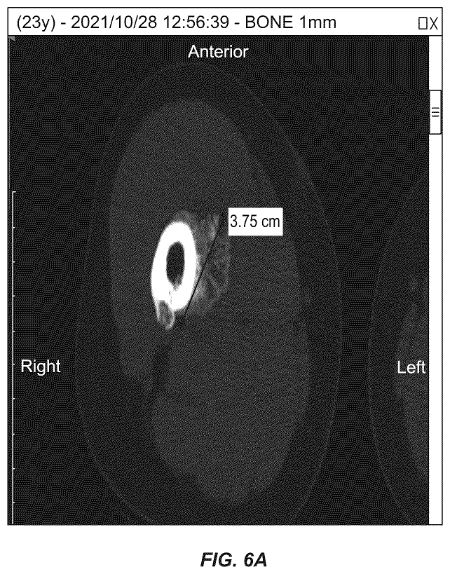

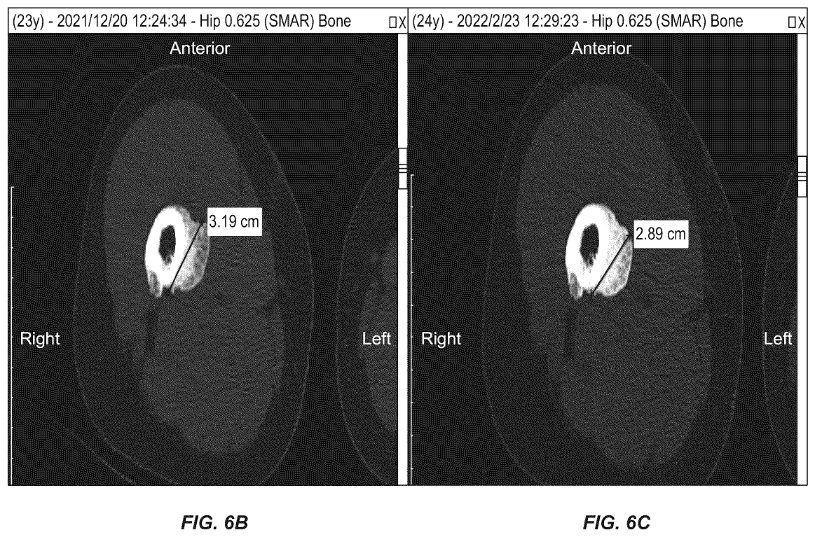

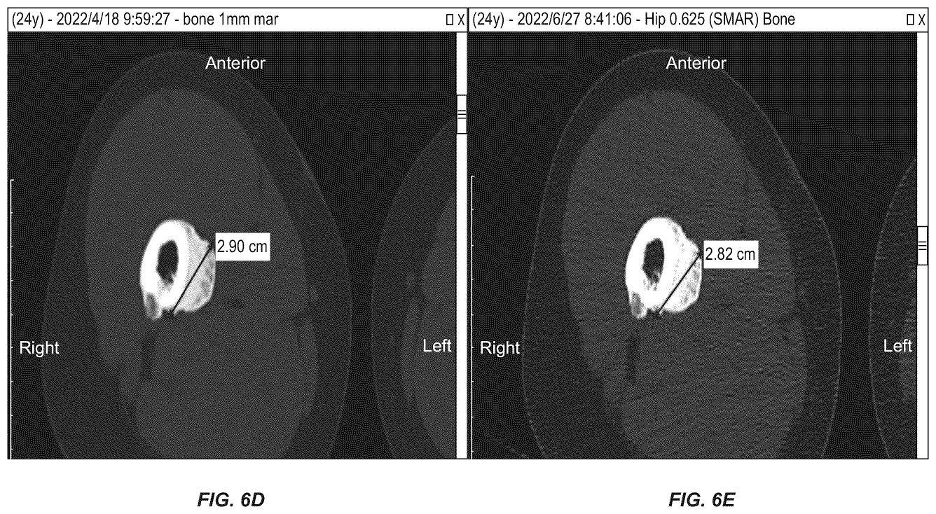

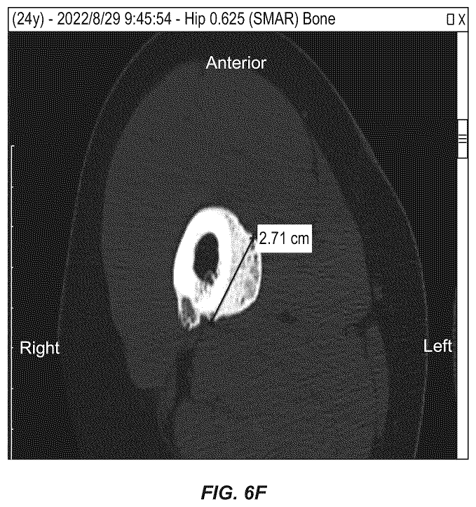

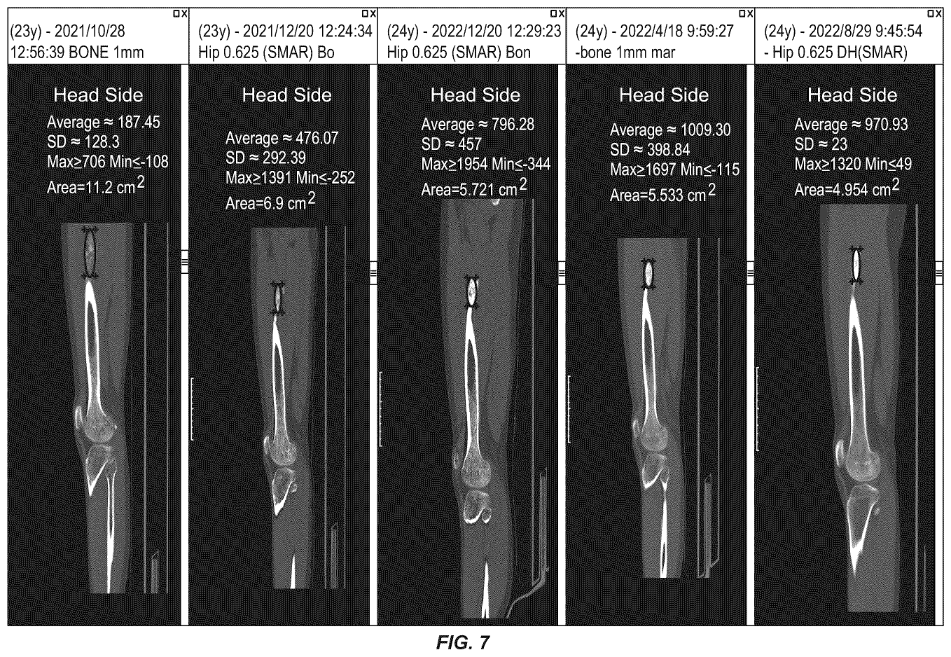

Participant SO1001 received chemotherapy after radical resection of osteosarcoma in left lower femur and for lung metastasis. After progression, Participant SO1001 received systemic chemotherapy with another drug and palliative resection of lung metastasis. Participant SO1001 received third-line chemotherapy and palliative resection of metastasis in right lower femur. After fourth-line chemotherapy, osteosarcoma recurred in the right femur and progressed. The participant met the inclusion criteria of the protocol and participated in the clinical study of ALMB-0168. Participant SO1001 was enrolled in September 2021 and treated with ALMB-0168. The right femur lesion continued to reduce (FIGS. 5 and 6B-6F) and Hu value continued to increase (FIG. 7), with the best response of PR. This participant was treated for more than 18 months with ALMB-0168 in the study.

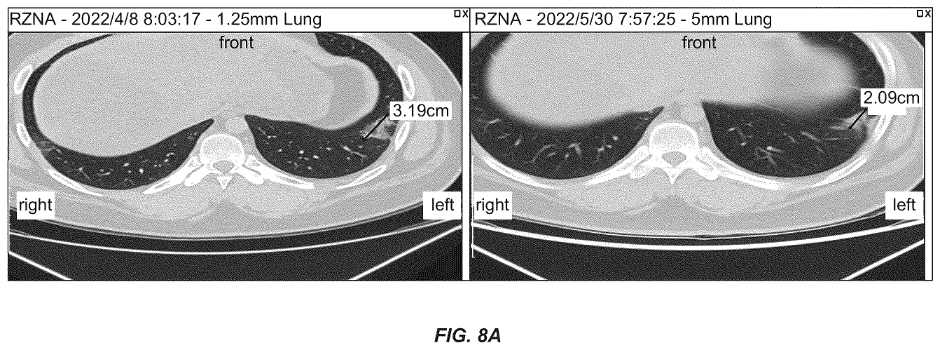

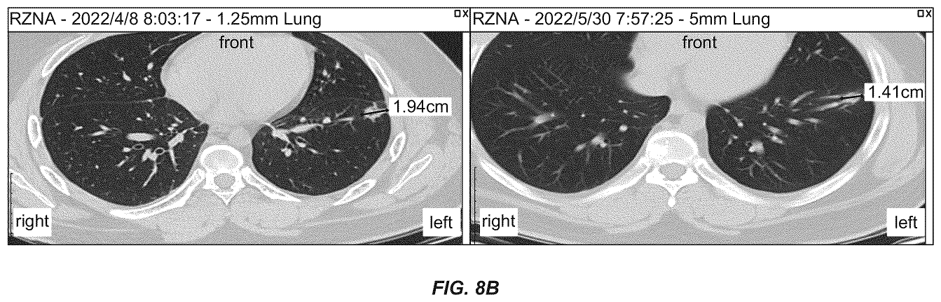

Regarding participant S02007, after surgery of the right distal femur osteosarcoma and after chemotherapy; received surgical resection of tibial metastasis 1 year later, and progressed after chemotherapy for lung lesions. This participant met the inclusion criteria of the protocol and participated in the clinical study of this drug, with the best response of PR. At baseline, participant S02007 had lesion 1 with a long diameter of 31.9 mm (FIG. 8A, left panel), and lesion 2 with a long diameter of 19.4 mm (FIG. 8B, left panel), totaling 51.3 mm. After treatment with ALMB-0168 in this study, the long diameter legion 1 reduced to 20.9 mm (FIG. 8A, right panel), and the long diameter legion 2 reduced to 14.1 mm (FIG. 8B, left panel), totaling 35.0 mm, which is 31.8% reduction from baseline, with the best response of PR.

These data demonstrated ALMB-0168 had surprisingly good efficacy and tolerable safety in patients with metastatic or unresectable osteosarcoma after failing the standard of care in a phase I/II dose-escalation trial.

TABLE 15
List of Abbreviations

| Abbreviations | Description |
|---|---|
| ADA | Anti-Drug Antibodies |
| AE | Adverse Event |
| ALB | Albumin |
| ALT | Alanine Aminotransferase |
| ALP | Alkaline Phosphatase |
| ANC | Absolute Neutrophil Count |
| APTT | Activated Partial Thromboplastin Time |
| AST | Aspartate Aminotransferase |
| AUC₀₋ₜ | Area under the Curve from Zero to the Time Point |
| AUC_{0-inf} | Area under the Curve from Zero to Infinite |
| BMD | Bone Mineral Density |
| BUN | Blood Urea Nitrogen |
| Ca | Calcium |
| Cl | Chlorine |
| CL | Clearance of Drug |
| Cr | Creatinine |
| CRF | Case Report Form |
| Cₘₐₓ | Maximum Concentration |
| Cx | Connexin |
| CxDy | Cycle X Day Y |
| DBil | Direct Bilirubin |
| DCR | Disease Control Rate |
| DLT | Dose Limited Toxicity |
| DNA | Deoxyribonucleic Acid |
| DXA | Dual Energy X-ray Absorptiometry |
| DF | Degree of Fluctuation |
| FAS | Full Analysis Set |
| GCP | Good Clinical Practice |
| GGT | Glutamyltransferase |
| h | Hour |
| Hb | Hemoglobin |
| HIV | Human Immunodeficiency Virus |
| INR | International Normalized Ratio |
| IHC | Immunohistochemistry |
| ITT | Intend to Treat |
| K | Potassium |
| LVEF | Left Ventricular Ejection Fraction |
| Mg | Magnesium |
| Min | Minute |
| MTD | Maximum Tolerated Dose |
| Na | Sodium |
| NAbs | Neutralizing Antibodies |
| NOAEL | No Observable Adverse Effect Level |
| NYHA | New York Heart Association |
| ORR | Objective Response Rate |
| P | Phosphorus |
| PFS | Progression-Free-Survival |
| PFSR | Progression-Free-Survival Rate |
| PLT | Platelet |
| PK | Pharmacokinetics |
| PT | Prothrombin Time |
| IHC | Immunohistochemistry |
| qPCR | Quantitative Polymerase Chain Reaction |
| RBC | Red Blood Cell |
| RNA | Ribonucleic Acid |
| RP2D | Recommended Phase 2 Dose |
| SAE | Serious Adverse Event |
| SOP | Standard Operation Procedure |
| SRE | Skeletal Related Events |
| SS | Safety Analysis Set |
| TBil | Total Bilirubin |
| TTR | Time to Response |
| t_{1/2} | Half-life |
| Tₘₐₓ | Time to Maximum Plasma Concentration |
| Vd | Volume of Distribution |
| ULN | Upper Limit of Normal |
| UREA | Urea |
| WBC | White Blood Cell |

TABLE 16
Sequence Table

| SEQ ID | | |
|---|---|---|
| NO | Description | Amino Acid Sequence |
| 1 | ALMB-0168 | GYTFTSYY |
| | HCDR1 | |
| 2 | ALMB-0168 | INPSNAGT |
| | HCDR2 | |
| 3 | ALMB-0168 | TREGNPYYTMNY |
| | HCDR3 | |
| 4 | ALMB-0168 | QSLLESDGKTY |
| | LCDR1 | |
| 5 | ALMB-0168 | LVS |
| | HCDR2 | |
| 6 | ALMB-0168 | WQGTHFPWT |
| | HCDR3 | |
| 7 | ALMB-0168 VH | EVQLVQSGAEVKKPGASVKVSCKASGYTFTSYYMYWVRQAPGQGLEWIGGINPSNAGTNFNEKFKNRATLT |
| | | VDKSTSTAYMELSSLRSEDTAVYYCTREGNPYYTMNYWGQGTLVTVSS |
| 8 | ALMB-0168 VL | DVVMTQSPLSLPVTIGQPASISCKSSQSLLESDGKTYLNWLQQRPGQSPRRLIYLVSKLDSGVPDRFSGSG |
| | | SGTDFTLKISRVEAEDVGVYYCWQGTHFPWTFGGGTKVEIK |
| 9 | Heavy chain of | EVQLVQSGAEVKKPGASVKVSCKASGYTFTSYYMYWVRQAPGQGLEWIGGINPSNAGTNFNEKFKNRATLT |
| | ALMB-0168 | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 10 | Light chain of | DVVMTQSPLSLPVTIGQPASISCKSSQSLLESDGKTYLNWLQQRPGQSPRRLIYLVSKLDSGVPDRFSGSG |
| | ALMB-0168 | SGTDFTLKISRVEAEDVGVYYCWQGTHFPWTFGGGTKVEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSPVTKS |
| | | FNRGEC |
| 11 | Heavy chain of | EVQLVQSGAEVKKPGASVKVSCKASGYTFTSYYMYWVRQAPGQGLEWIGGINPSNAGTNFNEKFKNRATLT |
| | Ab#L | VDKSTSTAYMELSSLRSEDTAVYYCTREGNPYYTMNYWGQGTLVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPAPIEKTISKAKGQPREPQVYTLPP |
| | | SRDELTKNQVSLTCLVKGFYPSDIAVEWESNGQPENNYKTTPPVLDSDGSFFLYSKLTVDKSRWQQGNVFS |
| | | CSVMHEALHNHYTQKSLSLSPGK |
| 12 | Heavy chain of | EVQLVQSGAEVKKPGASVKVSCKASGYTFTSYYMYWVRQAPGQGLEWIGGINPSNAGTNFNEKFKNRATLT |
| | Ab#P | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEFEGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 13 | Heavy chain of | EVQLVQSGAEVKKPGASVKVSCKASGYTFTSYYMYWVRQAPGQGLEWIGGINPSNAGTNFNEKFKNRATLT |
| | Ab#T | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEAAGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 14 | Epitope sequence | FLSRPTEKTI |
| | of Ab#H | |

1. A method of treating a bone cancer in a subject in need thereof, comprising administering to the subject at least one dose of an anti-connexin 43 (Cx43) antibody, or antigen binding fragment thereof, wherein the anti-Cx43 antibody comprises
a heavy chain variable region comprising HCDR amino acid sequences:
HCDR1: SEQ ID NO: 1;
HCDR2: SEQ ID NO: 2;
HCDR3: SEQ ID NO: 3;
and
a light chain variable region comprising LCDR amino acid sequences:
LCDR1: SEQ ID NO: 4;
LCDR2: SEQ ID NO: 5;
LCDR3: SEQ ID NO: 6.

2. The method of claim 1, wherein the at least one dose of the anti-Cx43 antibody, or antigen binding fragment thereof, is about 0.001 mg/kg to about 300 mg/kg, optionally wherein the at least one dose of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

3. (canceled)

4. The method of claim 1, wherein the anti-Cx43 antibody, or antigen binding fragment thereof, is administered about once every day to about once every 12 months: optionally wherein the anti-Cx43 antibody, or antigen binding fragment thereof, is administered about once every 2 weeks, about once every 3 weeks, about once every 4 weeks, or preferably once every 3 weeks.

5. (canceled)

6. (canceled)

7. The method of claim 1, wherein the anti-Cx43 antibody, or antigen binding fragment thereof, is administered intravenously, intradermally, intratumorally, intramuscularly, intraperitoneally, or subcutaneously; preferably administered intravenously;
optionally wherein each dose of the anti-Cx43 antibody, or antigen binding fragment thereof, is administered within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

8. (canceled)

9. (canceled)

10. The method of claim 1, wherein
(i) the anti-Cx43 antibody, or antigen binding fragment thereof, is administered in a pharmaceutically acceptable composition;
(ii) the anti-Cx43 antibody is a humanized antibody;
(iii) the anti-Cx43 antibody, or antigen binding fragment thereof, enhances Cx43 hemichannel opening in the subject.

11. The method of claim 1, wherein the anti-Cx43 antibody, or antigen binding fragment thereof, comprises a heavy chain variable region comprising amino acid sequence according to SEQ ID NO: 7, and/or a light chain variable region comprising amino acid sequence according to SEQ ID NO: 8.

12. The method of claim 1, wherein the anti-Cx43 antibody comprises a heavy chain comprising amino acid sequence according to any one of SEQ ID NOs: 9 and 11-13, and/or a light chain comprising amino acid sequence according to SEQ ID NO: 10.

13. (canceled)

14. (canceled)

15. The method of claim 1, wherein the bone cancer is osteosarcoma;
optionally wherein the osteosarcoma is
(i) resectable, or unresectable;
(ii) stage I-A, stage I-B, stage II-A, stage II-B, stage III, stage IV-A, or stage IV-B;
(iii) low grade, or high grade; and/or
(iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

16. (canceled)

17. The method of claim 1, wherein the subject had failed treatment with at least one standard care of osteosarcoma comprising chemotherapy, surgery, immune therapy, or radiotherapy, or a combination thereof,
optionally wherein the subject had failed one or more of treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or combination thereof.

18. (canceled)

19. The method of claim 1, wherein after treatment with the anti-Cx43 antibody, or antigen binding fragment thereof, the subject achieves:
(i) progression-free survival for at least 1 months, 2 months, 3 months, at least 4 months, at least 5 months, or at least 6 months;
(ii) improved overall survival by at least 1 month, at least 2 months, at least 3 months, at least 4 months, at least 5 months, at least 6 months;
(iii) improved EQ-5D score;
(iv) improved disease progression as assessed by PERCIST criteria, RECIST criteria, and/or ICDS criteria;
(v) improved pain score as assessed by NRS;
(vi) reduced tumor size by at least 1%, at least 2%, at least 3%, at least 5%, at least 8%, at least 10%, at least 15%, at least 20%, or at least 30%; and/or
(vii) complete or partial response.

20. (canceled)

21. A method of treating a bone cancer in a subject in need thereof, comprising administering to the subject at least one dose of an anti-connexin 43 (Cx43) antibody, or antigen binding fragment thereof, wherein the at least one dose of the anti-Cx43 antibody is about 0.001 mg/kg to about 300 mg/kg, about 1 mg/kg, about 3 mg/kg, about 6 mg/kg, about 12 mg/kg, about 18 mg/kg, about 24 mg/kg, about 30 mg/kg, or about 36 mg/kg.

22. (canceled)

23. The method of claim 21, wherein the anti-Cx43 antibody, or antigen binding fragment thereof, is administered about once every day to about once every 12 months, optionally wherein the anti-Cx43 antibody or antigen binding fragment thereof is administered about once every 2 weeks, about once every 3 weeks, or about once every 4 weeks: preferably about once every 3 weeks.

24. (canceled)

25. (canceled)

26. The method of claim 21, wherein the anti-Cx43 antibody, or antigen binding fragment thereof, is administered intravenously, intradermally, intratumorally, intramuscularly, intraperitoneally, subcutaneously, or locally; preferably administered intravenously;
optionally wherein each dose of the anti-Cx43 antibody, or antigen binding fragment thereof, is administered within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

27. (canceled)

28. (canceled)

29. The method of claim 21, wherein
(i) the anti-Cx43 antibody, or antigen binding fragment thereof, is administered in a pharmaceutically acceptable composition;
(ii) the anti-Cx43 antibody is a humanized antibody;
(iii) the anti-Cx43 antibody, or antigen binding fragment thereof, enhances Cx43 hemichannel opening in the subject.

30. The method of claim 21, wherein the anti-Cx43 antibody, or antigen binding fragment thereof, comprises
a heavy chain variable region comprising HCDR amino acid sequences:
HCDR1: SEQ ID NO: 1;
HCDR2: SEQ ID NO: 2;
HCDR3: SEQ ID NO: 3;
and
a light chain variable region comprising LCDR amino acid sequences:
LCDR1: SEQ ID NO: 4;
LCDR2: SEQ ID NO: 5;
LCDR3: SEQ ID NO: 6.

31. The method of claim 21, wherein the anti-Cx43 antibody, or antigen binding fragment thereof, comprises a heavy chain variable region comprising amino acid sequence according to SEQ ID NO: 7, and/or a light chain variable region comprising amino acid sequence according to SEQ ID NO: 8.

32. The method of claim 21, wherein the anti-Cx43 antibody comprises a heavy chain comprising amino acid sequence according to any one of SEQ ID NOs: 9 and 11-13, and/or a light chain comprising amino acid sequence according to SEQ ID NO: 10.

33. (canceled)

34. (canceled)

35. The method of claim 21, wherein the bone cancer is osteosarcoma,
optionally wherein the osteosarcoma is
(i) resectable, or unresectable;
(ii) stage I-A, stage I-B, stage II-A, stage II-B, stage III, stage IV-A, or stage IV-B;
(iii) low grade, or high grade; and/or
(iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

36. (canceled)

37. The method of claim 21, wherein the subject had failed treatment with at least one standard of care of osteosarcoma comprising chemotherapy, surgery, radiotherapy, or immune therapy, or the combination thereof,
optionally wherein the subject had failed one or more treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or combination thereof.

38. (canceled)

39. The method of claim 21, wherein after treatment with the anti-Cx43 antibody, or antigen-binding fragment thereof, the subject achieves:
(i) progression-free survival for at least 1 months, 2 months, 3 months, at least 4 months, at least 5 months, or at least 6 months;
(ii) improved overall survival by at least 1 month, at least 2 months, at least 3 months, at least 4 months, at least 5 months, at least 6 months;
(iii) improved EQ-5D score;
(iv) improved disease progression as assessed by PERCIST criteria, RECIST criteria, and/or ICDS criteria;
(v) improved pain score as assessed by NRS;
(vi) reduced tumor size by at least 1%, at least 2%, at least 3%, at least 5%, at least 8%, at least 10%, at least 15%, at least 20%, or at least 30%; and/or
(vii) complete or partial response.

40. (canceled)
